SEQUENCE LISTING (88 sequences)

SEQ ID NO: 1 - DNA, 24 bp, Artificial Sequence
  synthetic PCR amplification forward primer for 40F amplicon
tgtgtcttttacagagacgggttc

SEQ ID NO: 2 - DNA, 30 bp, Artificial Sequence
  synthetic PCR amplification reverse primer for 40F amplicon
gtttcttgctcacctgtagtccagctactc

SEQ ID NO: 3 - DNA, 25 bp, Artificial Sequence
  synthetic PCR amplification forward primer for 22F amplicon
cctatattcccagctacagctacac

SEQ ID NO: 4 - DNA, 34 bp, Artificial Sequence
  synthetic PCR amplification reverse primer for 22F amplicon
gtttcttgggtatatagtgatagtggtttgtttc

SEQ ID NO: 5 - DNA, 25 bp, Artificial Sequence
  synthetic PCR amplification forward primer for 41F amplicon
tcttttgactctaagtcccttagcc

SEQ ID NO: 6 - DNA, 32 bp, Artificial Sequence
  synthetic PCR amplification reverse primer for 41F amplicon
gtttcttgtttatagtccgctttttgtaaagg

SEQ ID NO: 7 - DNA, 24 bp, Artificial Sequence
  synthetic PCR amplification forward primer for 18F amplicon
atgatggctccagagtcatagaac

SEQ ID NO: 8 - DNA, 30 bp, Artificial Sequence
  synthetic PCR amplification reverse primer for 18F amplicon
gtttcttgtgcagatcaggaaggagaattg

SEQ ID NO: 9 - DNA, 25 bp, Artificial Sequence
  synthetic PCR amplification forward primer for 26F amplicon
gactgacactgttgtaataccaagg

SEQ ID NO: 10 - DNA, 32 bp, Artificial Sequence
  synthetic PCR amplification reverse primer for 26F amplicon
gtttcttggtttcaaacattacaagatcaagg

SEQ ID NO: 11 - DNA, 23 bp, Artificial Sequence
  synthetic PCR amplification forward primer for 27F amplicon
ctgatgagggacaggaatctcac

SEQ ID NO: 12 - DNA, 28 bp, Artificial Sequence
  synthetic PCR amplification reverse primer for 27F amplicon
gtttcttatgacccagggacaggtacag

SEQ ID NO: 13 - DNA, 25 bp, Artificial Sequence
  synthetic PCR amplification forward primer for 30F amplicon
ggagtattgctaggagggagttttc

SEQ ID NO: 14 - DNA, 32 bp, Artificial Sequence
  synthetic PCR amplification reverse primer for 30F amplicon
gtttcttcgctatatgggtagtcactatctgg

SEQ ID NO: 15 - DNA, 25 bp, Artificial Sequence
  synthetic PCR amplification forward primer for 47F amplicon
tttggttaaggccctaaatttgaac

SEQ ID NO: 16 - DNA, 32 bp, Artificial Sequence
  synthetic PCR amplification reverse primer for 47F amplicon
gtttctttttctgcatttttatagtgctttcc

SEQ ID NO: 17 - DNA, 25 bp, Artificial Sequence
  synthetic PCR amplification forward primer for 34F amplicon
gttataaaagatgcaactgctcagg

SEQ ID NO: 18 - DNA, 32 bp, Artificial Sequence
  synthetic PCR amplification reverse primer for 34F amplicon
gtttcttgtagctactgattctggttcctctg

SEQ ID NO: 19 - DNA, 25 bp, Artificial Sequence
  synthetic PCR amplification forward primer for 45F amplicon
aaggtctgagataagctccagaatc

SEQ ID NO: 20 - DNA, 31 bp, Artificial Sequence
  synthetic PCR amplification reverse primer for 45F amplicon
gtttcttaccttagagttcggtgtcatgaag

SEQ ID NO: 21 - DNA, 27 bp, Artificial Sequence
  synthetic PCR amplification forward primer for 54F amplicon
ctaagtgttaaggacacagactgaagg

SEQ ID NO: 22 - DNA, 34 bp, Artificial Sequence
  synthetic PCR amplification reverse primer for 54F amplicon
gtttcttgagaccttacaggaacagaagaatatc

SEQ ID NO: 23 - DNA, 26 bp, Artificial Sequence
  synthetic PCR amplification forward primer for 2F amplicon
tcatcaggttactaggcaatattagg

SEQ ID NO: 24 - DNA, 29 bp, Artificial Sequence
  synthetic PCR amplification reverse primer for 2F amplicon
gtttcttctctgtccctgaccaggtctac

SEQ ID NO: 25 - DNA, 21 bp, Artificial Sequence
  synthetic PCR amplification forward primer for 25F amplicon
cacactccttggtgacagtgc

SEQ ID NO: 26 - DNA, 27 bp, Artificial Sequence
  synthetic PCR amplification reverse primer for 25F amplicon
gtttcttgtcaggtcagtccgtggttc

SEQ ID NO: 27 - DNA, 25 bp, Artificial Sequence
  synthetic PCR amplification forward primer for 32F amplicon
ggaggttgactaaggatctacacag

SEQ ID NO: 28 - DNA, 32 bp, Artificial Sequence
  synthetic PCR amplification reverse primer for 32F amplicon
gtttcttctactacagggacatcacacacaac

SEQ ID NO: 29 - DNA, 25 bp, Artificial Sequence
  synthetic PCR amplification forward primer for 46F amplicon
ccgttattaaaaagtctcacgtttg

SEQ ID NO: 30 - DNA, 32 bp, Artificial Sequence
  synthetic PCR amplification reverse primer for 46F amplicon
gtttcttatatctaacctctcctcaggtttcc

SEQ ID NO: 31 - DNA, 25 bp, Artificial Sequence
  synthetic PCR amplification forward primer for 21F amplicon
tacccaggtgtaagatcttgaaaag

SEQ ID NO: 32 - DNA, 32 bp, Artificial Sequence
  synthetic PCR amplification reverse primer for 21F amplicon
gtttcttaggaaacctctactcatgctgaaag

SEQ ID NO: 33 - DNA, 28 bp, Artificial Sequence
  synthetic PCR amplification forward primer for 52F amplicon
cagctaatttttctgtttttagtacagg

SEQ ID NO: 34 - DNA, 27 bp, Artificial Sequence
  synthetic PCR amplification reverse primer for 52F amplicon
gtttctttgcagtcaagaacccatcac

SEQ ID NO: 35 - DNA, 20 bp, Artificial Sequence
  synthetic PCR amplification forward primer for 36F amplicon
gggcattcaggaccactagg

SEQ ID NO: 36 - DNA, 29 bp, Artificial Sequence
  synthetic PCR amplification reverse primer for 36F amplicon
gtttcttgttcagagcgtctcttggtttc

SEQ ID NO: 37 - DNA, 20 bp, Artificial Sequence
  synthetic PCR amplification forward primer for D17S250f amplicon
ggaagaatcaaatagacaat

SEQ ID NO: 38 - DNA, 24 bp, Artificial Sequence
  synthetic PCR amplification reverse primer for D17S250f amplicon
gctggccatatatatatttaaacc

SEQ ID NO: 39 - DNA, 27 bp, Artificial Sequence
  synthetic PCR amplification forward primer for 89F amplicon
tagtcaccttgttcagcacctaatatc

SEQ ID NO: 40 - DNA, 30 bp, Artificial Sequence
  synthetic PCR amplification reverse primer for 89F amplicon
gtttcttgtgactatccactctttgccttg

SEQ ID NO: 41 - DNA, 27 bp, Artificial Sequence
  synthetic PCR amplification forward primer for 78F amplicon
caaagagtgaaacagactatcgacttc

SEQ ID NO: 42 - DNA, 35 bp, Artificial Sequence
  synthetic PCR amplification reverse primer for 78F amplicon
gtttcttaacctttagatttacagaaaaattgagc

SEQ ID NO: 43 - DNA, 27 bp, Artificial Sequence
  synthetic PCR amplification forward primer for 84F amplicon
aggtgtctgagaataaagaagatgaag

SEQ ID NO: 44 - DNA, 29 bp, Artificial Sequence
  synthetic PCR amplification reverse primer for 84F amplicon
gtttcttatggattcctggtgagatgttg

SEQ ID NO: 45 - DNA, 25 bp, Artificial Sequence
  synthetic PCR amplification forward primer for 88F amplicon
aattttcagtctctgagtgtgatcc

SEQ ID NO: 46 - DNA, 31 bp, Artificial Sequence
  synthetic PCR amplification reverse primer for 88F amplicon
gtttcttcatttgcgagcaatttctctttag

SEQ ID NO: 47 - DNA, 27 bp, Artificial Sequence
  synthetic PCR amplification forward primer for 91F amplicon
aattcagaggtctatgtgatagtaggc

SEQ ID NO: 48 - DNA, 32 bp, Artificial Sequence
  synthetic PCR amplification reverse primer for 91F amplicon
gtttcttgaactcagttttcaaggtttctttg

SEQ ID NO: 49 - DNA, 27 bp, Artificial Sequence
  synthetic PCR amplification forward primer for 58F amplicon
gtaagtaaatcaatgaatgtggttgtg

SEQ ID NO: 50 - DNA, 34 bp, Artificial Sequence
  synthetic PCR amplification reverse primer for 58F amplicon
gtttcttataaatttttattggattttcgtttgg

SEQ ID NO: 51 - DNA, 27 bp, Artificial Sequence
  synthetic PCR amplification forward primer for 64F amplicon
gtaatcaccatcaatttggcaatttac

SEQ ID NO: 52 - DNA, 33 bp, Artificial Sequence
  synthetic PCR amplification reverse primer for 64F amplicon
gtttcttgactaagggaggagaatcactagaac

SEQ ID NO: 53 - DNA, 28 bp, Artificial Sequence
  synthetic PCR amplification forward primer for 75F amplicon
catgagttcaattgtttttatttttagc

SEQ ID NO: 54 - DNA, 32 bp, Artificial Sequence
  synthetic PCR amplification reverse primer for 75F amplicon
gtttcttcatttctgagataagggttcaaatg

SEQ ID NO: 55 - DNA, 27 bp, Artificial Sequence
  synthetic PCR amplification forward primer for 101F amplicon
ttatgaactcaagaagctaaagaaacg

SEQ ID NO: 56 - DNA, 34 bp, Artificial Sequence
  synthetic PCR amplification reverse primer for 101F amplicon
gtttcttgggagataaaatggaaaacattaaaac

SEQ ID NO: 57 - DNA, 23 bp, Artificial Sequence
  synthetic PCR amplification forward primer for D5S346f amplicon
actcactctagtgataaatcggg

SEQ ID NO: 58 - DNA, 25 bp, Artificial Sequence
  synthetic PCR amplification reverse primer for D5S346f amplicon
agcagataagacagtattactagtt

SEQ ID NO: 59 - DNA, 27 bp, Artificial Sequence
  synthetic PCR amplification forward primer for 66F amplicon
acatgtacattcagttcactgttaagc

SEQ ID NO: 60 - DNA, 34 bp, Artificial Sequence
  synthetic PCR amplification reverse primer for 66F amplicon
gtttctttagctttgttctagttttgtgtgtgtg

SEQ ID NO: 61 - DNA, 27 bp, Artificial Sequence
  synthetic PCR amplification forward primer for 87F amplicon
tacatgaaattctcaatgattacaacg

SEQ ID NO: 62 - DNA, 32 bp, Artificial Sequence
  synthetic PCR amplification reverse primer for 87F amplicon
gtttcttaagatctattccatcccattgactc

SEQ ID NO: 63 - DNA, 26 bp, Artificial Sequence
  synthetic PCR amplification forward primer for 57F amplicon
ccgaatcttaaattgaaaacacaaag

SEQ ID NO: 64 - DNA, 32 bp, Artificial Sequence
  synthetic PCR amplification reverse primer for 57F amplicon
gtttctttttttagtagaaatggggtttcacc

SEQ ID NO: 65 - DNA, 26 bp, Artificial Sequence
  synthetic PCR amplification forward primer for 104F amplicon
agttacgacaatcaaaaatgtctctg

SEQ ID NO: 66 - DNA, 31 bp, Artificial Sequence
  synthetic PCR amplification reverse primer for 104F amplicon
gtttcttgagatgcctagaccactgattctc

SEQ ID NO: 67 - DNA, 27 bp, Artificial Sequence
  synthetic PCR amplification forward primer for 81F amplicon
gtgaactgtgtttctgtcactacactc

SEQ ID NO: 68 - DNA, 34 bp, Artificial Sequence
  synthetic PCR amplification reverse primer for 81F amplicon
gtttctttacaaaaatcatggtttagttttctcc

SEQ ID NO: 69 - DNA, 28 bp, Artificial Sequence
  synthetic PCR amplification forward primer for 103F amplicon
gggcagtattaaaaactatagaataccc

SEQ ID NO: 70 - DNA, 32 bp, Artificial Sequence
  synthetic PCR amplification reverse primer for 103F amplicon
gtttctttacactcttgtgcattttccttttc

SEQ ID NO: 71 - DNA, 26 bp, Artificial Sequence
  synthetic PCR amplification forward primer for 83F amplicon
cagtgtctcattcatctttgtcattc

SEQ ID NO: 72 - DNA, 34 bp, Artificial Sequence
  synthetic PCR amplification rverse primer for 83F amplicon
gtttcttcaaaaactacaaaatgtcttaatggag

SEQ ID NO: 73 - DNA, 21 bp, Artificial Sequence
  synthetic PCR amplification forward primer for BAT26fwd amplicon
tgactacttttgacttcagcc

SEQ ID NO: 74 - DNA, 22 bp, Artificial Sequence
  synthetic PCR amplification reverse primer for BAT26fwd amplicon
aaccattcaacatttttaaccc

SEQ ID NO: 75 - DNA, 20 bp, Artificial Sequence
  synthetic PCR amplification forward primer for D2S123f amplicon
aaacaggatgcctgccttta

SEQ ID NO: 76 - DNA, 20 bp, Artificial Sequence
  synthetic PCR amplification reverse primer for D2S123f amplicon
ggactttccacctatgggac

SEQ ID NO: 77 - DNA, 27 bp, Artificial Sequence
  synthetic PCR amplification forward primer for 105F amplicon
ttaccttaacattcagtcttcctcttg

SEQ ID NO: 78 - DNA, 34 bp, Artificial Sequence
  synthetic PCR amplification reverse primer for 105F amplicon
gtttctttagatatgccacttttgtcatctacag

SEQ ID NO: 79 - DNA, 27 bp, Artificial Sequence
  synthetic PCR amplification forward primer for 74F amplicon
taacaagggaatgtaaaggaacttatg

SEQ ID NO: 80 - DNA, 35 bp, Artificial Sequence
  synthetic PCR amplification forward primer for 74F amplicon
gtttcttttatttagtccagatttaatgacaaagg

SEQ ID NO: 81 - DNA, 27 bp, Artificial Sequence
  synthetic PCR amplification forward primer for 102F amplicon
ttggtattctattatagcagcctgaac

SEQ ID NO: 82 - DNA, 35 bp, Artificial Sequence
  synthetic PCR amplification reverse primer for 102F amplicon
gtttcttcattacacatacttattaccaccaggac

SEQ ID NO: 83 - DNA, 27 bp, Artificial Sequence
  synthetic PCR amplification forward primer for 107F amplicon
ctctcatgacctagctaaaaatgattc

SEQ ID NO: 84 - DNA, 34 bp, Artificial Sequence
  synthetic PCR amplification reverse primer for 107F amplicon
gtttcttgcccagacttttatttcttattttgtc

SEQ ID NO: 85 - DNA, 20 bp, Artificial Sequence
  synthetic PCR amplification forward primer for Bat25f amplicon
tcgcctccaagaatgtaagt

SEQ ID NO: 86 - DNA, 21 bp, Artificial Sequence
  synthetic PCR amplification reverse primer for Bat25f amplicon
tctgcattttaactatggctc

SEQ ID NO: 87 - DNA, 12 bp, Artificial Sequence
  polyguanine (Poly-G) tract allele
nnnggggggnnn

SEQ ID NO: 88 - DNA, 10 bp, Artificial Sequence
  polyguanine (Poly-G) tract allele
nnnggggnnn

(19) United States
(12) Patent Application Publication
(10) Pub. No.: US 2011/0003289 A1
(43) Pub. Date: Jan. 6, 2011

(54) METHOD FOR DETECTION OF PRE-NEOPLASTIC FIELDS AS A CANCER BIOMARKER IN ULCERATIVE COLITIS

(75) Inventors: Jesse Salk, Seattle, WA (US); Stephen Salipante, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of Washington, Seattle, WA (US)

(21) Appl. No.: 12/726,287

(22) Filed: Mar. 17, 2010

Related U.S. Application Data
(60) Provisional application No. 61/160,999, filed on Mar. 17, 2009.

(51) Int. Cl. C12Q 1/68; C07H 21/00

(52) U.S. Cl. 435/6; 536/24.33

(57) ABSTRACT

Among other aspects, the present invention provides biomarkers and methods of identifying precancerous fields in a subject in need thereof. Methods of diagnosing and for providing a prognosis for a subject with an increased risk of developing cancer are also provided, along with methods of determining surgical margins for a tumor or tissue resection procedure. Additionally, reagents and kits are provided for the practice of the methods disclosed herein.

### CROSS-REFERENCES TO RELATED APPLICATIONS

The present application claims the benefit of U.S. Provisional Application No. 61/160,999 filed Mar. 17, 2009, expressly incorporated herein by reference in its entirety for all purposes.

### STATEMENT AS TO RIGHTS TO INVENTIONS MADE UNDER FEDERALLY SPONSORED RESEARCH OR DEVELOPMENT

This invention was made with Government support of Grant Nos. DPI OD003278 and 2R01 CA068124-11A1, awarded by the NIH. The Government has certain rights in this invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Polyguanine (Poly-G) tracts (equivalent to Poly-C tracts, depending on the nucleic acid strand being examined) are mononucleotide repeats found in the genome of many eukaryotes, including worms, rodents, and humans. Since these mononucleotide repeats, as well as other nucleotide repeats such as di- and trinucleotide repeats, lack sequence complexity, nucleic acid polymerases frequently alter the length of these tracts when they “slip” on the sequence during genomic replication, causing hairpin structures that lead to nucleotide tract lengthening or shortening. When a slippage event occurs in the middle of a coding region, protein sequences can be altered, resulting in potentially drastic phenotypic effects as seen in a number of molecularly characterized diseases. As such, a number of these repeat tracts have been well characterized (for review see, Sinden R. R., Am J Hum Genet. 1999 February; 64(2):346-53). Conversely, when an alteration occurs in a repeat tract located in an extragenic location, the phenotypic consequences are generally non-existent.

Recently, it was shown that given the high frequency of somatic nucleotide insertions and deletions at poly-G tracts (Boyer et al., Hum Mol Genet. 2002 Mar. 15; 11(6):707-13), these sequences serve as markers useful for inferring cell lineages (Salipante and Horwitz, Curr Top Dev Biol. 2007; 79:157-84; Salipante et al., Genetics. 2008 February; 178(2):967-77). Specifically, it was shown that characterization of the length of Poly-G tracts could be used to reconstruct the lineage of cultured NIH 3T3 murine cells.

The human colon is divided into discrete replicative units known as crypts. It is generally accepted that a population of somewhere between two and ten colonic stem cells reside at the base of these crypts. Over the course of approximately ten days in humans, daughter cells resulting from division within this stem cell population move luminally to populate the remainder of the crypt, first as transiently amplifying cells then as terminally differentiated colonocytes prior to being sloughed off. It is believed by many that a major reason for this hierarchical structure is that it acts as an antineoplastic defense. The more cell divisions of long-lived stem cells that occur, the more opportunities there are for mutations to accumulate that are transmissible to another generation and the greater the chance that one or more of these mutations will result an oncogenic fitness advantage. Mutations that wind up in terminally differentiated cells are evolutionary dead ends since they are not inheritable by another generation of daughters.

Large, laterally-spreading clonal expansions in the colon are abnormal and represent a divergence from regular colonic homeostatic mechanisms. Although crypt units seem to occasionally reproduce themselves in normal human colon by duplication of their stem cell niche and subsequent bifurcation, the normal extent of this is very limited. Studies that have tracked lineages of crypts using stainable spontaneous mutations in mitochondrial cyclooxygenase genes indicate that the size of new crypt patches derived from a common ancestor in normal colon during the course of one's life is generally fewer than 10 with a mean number of about four crypts (Greaves et al 2006, PNAS103(3) pp. 714-9).

Inflammatory bowel disease, including ulcerative colitis (UC) are Crohn's disease (CD), are disorder characterized by chronic inflammation of the colon mucosa affecting approximately 1 million individuals in the United States alone (Hanauer S. B., Inflamm Bowel Dis. 2006 January; 12 Suppl 1:S3-9). Ulcerative colitis is a chronic disease of the large bowel, while Crohn's disease is variably found throughout the entire GI tract. Both diseases significantly increase an individual's risk of developing colon or colorectal cancer (for review see, Risques R A et al., Curr Opin Gastroenterol. 2006 July; 22(4):382-90). Both UC and CD patients must be under routine surveillance so that when a cancer arises it can be detected at an early stage and surgically removed. Surveillance consists of taking about 30 to 60 biopsies annually along the length of the colon with the hope of randomly sampling a cancer if it exists. Such measures are expensive, uncomfortable, insensitive to small lesions, and only useful for detecting a cancer after it arises.

In general terms, cancer is a diagnosis based on morphological abnormality of a tissue either at the macroscopic or microscopic level. It has been hypothesized that some cancers may arise from a large background or field of morphologically normal, yet genetically abnormal cells (for review see, Braakhuis B J et al., Cancer Res. 2003 Apr. 15; 63(8):1727-30). In theory, if it were possible to detect this field, it should be possible to intervene on the process before a cancer itself arises. In particular, a number of diseases have been identified as predisposing a subject to an increased risk of developing cancer. Prominently, a number of inflammatory diseases including ulcerative colitis, Crohn's disease, pelvic inflammatory disease, hepatitis, pancreatitis, and cholangitis, have been linked to an increased risk of developing cancer. Patients diagnosed with these diseases often undergo surveillance, frequently consisting of taking a large number of biopsies on a routine basis, in order to detect the early signs of cancer progression.

As such, there is a need in the art for less-obtrusive, yet highly effective methods of detecting precancerous or preneoplastic fields before the onset of cancer progression. The present invention satisfies these and other needs by providing novel biomarkers and methods for the detection of preneoplastic fields, methods of providing a prognosis for a subject in need thereof, and methods of determining and evaluating surgical margins in a subject in need of a tumor of tissue resection.

### BRIEF SUMMARY OF THE INVENTION

In one aspect, the present invention provides methods of identifying the presence of a precancerous field in a subject. In certain embodiments, the methods comprise the detection of a somatic genetic alteration that marks a clonal expansion event in a biological sample from the subject. In one embodiment, the method comprises detecting a neutral genetic change that is associated with a clonal expansion event.

In another aspect, the present invention provides a method of identifying the presence of a precancerous field in a subject, the method comprising detecting a discreet change in the size of a repeated genomic tract in a biological sample from the subject. In one embodiment, the methods provided herein comprise the detection of a discreet change in the size of a mononucleotide repeat tract. In other embodiments, the nucleotide repeat tract may comprise a dinucleotide repeat, a trinucleotide repeat, or a higher-order nucleotide repeat sequence. In specific embodiments, the methods comprise the detection of one or more marker loci selected from those found in Table 1.

In another aspect, the present invention provides methods of identifying the presence of a precancerous field in a subject that has been identified as having an increased risk of developing cancer. In certain embodiments, the methods provided comprise detection of a somatic genetic alteration that marks a clonal expansion event in a biological sample from the subject. In certain embodiments, the methods comprise detection of a discreet change in the size of a repeated genomic tract in a biological sample from the subject. In some embodiments, the subject has been chronically exposed to a cancerous risk factor or carcinogen, for example, asbestos, tobacco smoke, ultraviolet radiation, radioactive decay, and the like. In other embodiments, the subject has been diagnosed with a disease or condition associated with an increased risk of developing cancer, for example, ulcerative colitis, Crohn's disease, post-menopausal bleeding, oral leukoplakia, and the like. In yet other embodiments, the subject has been diagnosed with a gastrointestinal disorder or chronic inflammatory disease or condition.

In another aspect, the invention provides methods of providing a prognosis for a subject diagnosed with a disease or condition that is associated with an increased risk of developing cancer. In some embodiments, the method comprises detecting a somatic genetic alteration that marks a clonal expansion event in a biological sample from the subject, wherein the somatic genetic alteration indicates that the subject has a further elevated risk of developing cancer. In certain embodiments, the method comprises detecting a discreet change in the size of a repeated nucleotide tract in a biological sample from the subject, wherein the discreet change in the size of the repeated nucleotide tract indicates that the subject has a further elevated risk of developing cancer. In specific embodiments, the repeated nucleotide tract is selected from those found in Table 1.

In another aspect, the present invention provides methods of assigning a course of treatment to a subject diagnosed with a disease or condition that is associated with an increased risk of developing cancer. In some embodiments, the method comprises detecting a somatic genetic alteration that marks a clonal expansion event in a biological sample from the subject, and assigning a treatment. In certain embodiments, the method comprises detecting a discreet change in the size of a repeated nucleotide tract in a biological sample from the subject and assigning a treatment. In some embodiments the course of treatment comprises performing additional tests to further diagnose or characterize the disease or condition. In other embodiments, the course of treatment comprises more frequent surveillance. In yet other embodiments, the course of treatment comprises removing part or all of a precancerous field from the subject.

In another aspect, the present invention provides less obtrusive methods of monitoring a disease or condition. In some embodiments, the methods comprise monitoring a subject diagnosed with a disease or condition associated with an increased risk of developing cancer. In other embodiments, the methods comprise monitoring a subject who has been chronically exposed to a cancerous risk factor or carcinogen, and thus have an increased risk of developing cancer. In certain embodiments, the methods comprise performing a fewer number of biopsies than employed in the current methods of cancer surveillance.

In another aspect, the present invention provides methods of determining the margins of a precancerous field that are at the greatest risk of progression. In some embodiments, the methods comprise detecting a somatic genetic alteration that marks a clonal expansion event in a plurality of biopsies from a subject. In certain embodiment, the methods comprise detecting a discreet change in the size of a repeated nucleotide tract in a plurality of biopsies from the subject.

In another aspect, the present invention provides methods of determining the surgical margins for a tumor or tissue resection procedure. In some embodiments, the methods comprise detecting a somatic genetic alteration that marks a clonal expansion event in a plurality of biopsies from a subject. In a certain embodiment, the methods comprise detecting a discreet change in the size of a repeated nucleotide tract in a plurality of biopsies from the subject. In some embodiments, the tissue resection is for removal of at least a portion of a precancerous field.

In another embodiment, the present invention provides methods of assessing the adequacy of surgical margins used for a tumor or tissue resection. In one embodiment, the methods comprise detecting a somatic genetic alteration that marks a clonal expansion event in a biological sample isolated from a location proximal to the surgical resection. In another embodiment, the method comprises detecting a discreet change in the size of a repeated nucleotide tract in a biological sample isolated from a location proximal to the surgical resection. In certain embodiments, the nucleotide tract is selected from those found in Table 1. In a particular embodiment, the tissue resection is a surgical resection of a cancer or precancerous field.

In another aspect, the present invention provides biomarkers for the detection of a precancerous field in a subject. In certain embodiments, these biomarkers comprise somatic genetic alterations that mark a clonal expansion event. In some embodiments, these biomarkers comprise a repeated nucleotide tract. In a particular embodiment, the biomarkers are genomic loci selected from those found in Table 1.

In another aspect, the present invention provides methods of detecting the biomarkers of the invention. In certain embodiments, the methods comprise sequencing a marker loci from a biological sample from a subject. In some embodiments, the methods comprise determining the length of a repeated nucleotide tract. In particular embodiments, the methods comprise amplifying a marker loci and determining the length of a repeated nucleotide tract.

In another aspect, the present invention provides kits and reagents useful for detecting the biomarkers of the invention and for practicing the methods provided herein. In some embodiments, primers are provided for amplifying a marker loci, a somatic genetic alteration, or a nucleotide repeat tract. In other embodiments, hybridization probes are provided herein for detecting the biomarkers of the invention. In other embodiments, kits are provided which may comprise a primer or hybridization probe of the invention.

### BRIEF DESCRIPTION OF THE DRAWINGS

FIG. 1. Schematic overview of and example of data generated from a PCR method for determining the length of Poly-G tract alleles at a marker loci in a subject. Genotypes are called based on the major peaks in the electropherograms shown in the upper left corner. The minor side peaks in the electropherograms represent artifacts introduced by PCR amplification of a mononucleotide repeat and should not be confused with the broad distribution of multiple peaks of electropherograms that indicate traditionally-defined microsatellites instability such as found in microsatellite unstable (MSI) sporadic colorectal cancers bearing deficiencies in MSH or MLH gene expression.

FIG. 2. POLY-G Slippage Assay Distinguishes the UC Progressors versus Non-Progressors. The 2^{nd} pilot PolyG study verified that mutations were present in the non-dysplastic colonic mucosa in all of the UC Progressors (11/11) while it was present in only a single biopsy from ⅛ UC Non-Progressors. Data graphed as (A) total number of mutants detected and (B) percentage mutant biopsies. The red bars represent clonally expanded slippage events that were confirmed on a single run. Blue bars represent clonally expanded slippage events that were ambiguous on the first run and repeated on a second run (˜90% of these were confirmed as true events while ˜10% were artifacts). Non-UC=cases 1-3; UC-NP=cases 4-10,14,15; UC-Prog=cases 7, 11-13, 17-22.

FIG. 3. ROC analysis of the performance of the poly-G test for prognosis of UC progression.

FIG. 4. Assessment of the possible correlation between the frequency of mutant markers found (right column of each pair) and the duration of the disease (left column of each pair) in the UC progressor cohort.

FIG. 5. Assessment of the possible correlation between the frequency of mutant markers found (right column of each pair) and the age of the subject (left column of each pair) in the UC progressor cohort.

FIG. 6. Assessment of the possible correlation between the frequency of mutant markers found (right column of each pair) and the severity of the disease (left column of each pair) in the UC progressor cohort.

FIG. 7. Assessment of the possible correlation between the frequency of mutant markers found (right column of each pair) and the PSC status of the subject (left column of each pair) in the UC progressor cohort.

FIG. 8. Assessment of the possible correlation between the frequency of mutant markers found (right column of each pair) and the cancer status of the subject (left column of each pair) in the UC progressor cohort.

FIG. 9. Assessment of the possible correlation between the frequency of mutant markers found (right column of each pair) and the sex of the subject (left column of each pair) in the UC progressor cohort.

FIG. 10. Graph of the cumulative fraction of mutations and total number of biopsies from UC Progressors as a function of distance from the dysplasia.

FIG. 11. Chart of the total number of mutants, total number of confirmed mutants, and total number of biopsies from UC Progressors as a function of distance from the dysplasia.

FIG. 12. Mapping studies of poly-G slippage in biopsies taken throughout the colons of 10 UC Progressors. There are 3-8 biopsies tested per patient from different locations in the colon. The X axis represents normalized units of distance to dysplasia with the furthest distance being defined as 1.0. The Y axis represents the number of poly-G slippage mutations discovered per biopsy. While most of the biopsies revealed mutations, approximately one third did not using the limited number of current primer sets. Distance to dysplasia did not influence whether biopsies had mutations or not.

FIG. 13. Representation of the percentage of the total number of slippage events occurring at each marker loci.

FIG. 14. Representation of the percentage of successful genotypes obtained from each marker loci.

FIG. 15. Comparison between the success rate of obtaining a viable genotype and the percentage of the total number of slippage events identified for each marker loci.

FIG. 16. Key for interpreting raw sequencing data for a slippage event at a given marker loci.

FIG. 17. Example of data for Patient 9 at amplicon (marker loci) 66F showing no slippage events in either allele in the stroma or epithelium for any of the four biopsies.

FIG. 18. Example of data for Patient 1 at amplicon (marker loci) 66F showing an isolated slippage event in the epithelium from a single biopsy.

FIG. 19. Example of data for Patient 7 at amplicon (marker loci) 18F showing an isolated slippage event in the stroma from a single biopsy.

FIG. 20. Example of data for Patient 6 at amplicon (marker loci) 87F showing identical slippage events in an epithelial and stromal pair from a single biopsy.

FIG. 21. Example of data showing all types of slippage events.

FIG. 22. Example of data for Patient 11 at amplicon (marker loci) 18F showing two different types of slippage events in two biopsies from the epithelium.

FIG. 23. Example of data for Patient 13 at amplicon (marker loci) 41F showing slippage events in two epithelial/stromal pairs. One slippage event is the same in the epithelial/stromal pair for biopsy 3, while the other is different in the epithelial/stromal pair for biopsy 2.

FIG. 24. Example of data for Patient 13 at amplicon (marker loci) 104F showing the same marker allele in two epithelial biopsies taken at locations 19 cm apart.

FIG. 25. Example of data for Patient 19 at amplicon (marker loci) 66F showing slippage events in three epithelial biopsies. Identical marker alleles are found in biopsies 3 and 4, which were about 19 cm apart. A different set of marker alleles is seen in biopsy 1, which was located about 42 cm from biopsy 3 and about 61 cm from biopsy 4.

FIG. 26. (A) Example of data for Patient 21 at amplicons (marker loci) 66F and 87F showing slippage events in two marker alleles in epithelial biopsy 4 and one identical marker allele in biopsy 3. (B) Schematic of the data obtained for Patient 21, wherein biopsy 3 is located in a first neo-plastic field and contains a single slippage event marker allele and biopsy 4 is located in both the first and a second pre-neoplastic field, which overlap, and contains two slippage event marker alleles.

FIG. 27. (A) Example of data from Patient 16 at amplicons (marker loci) 18F, 58, and 66F showing a complex pattern of marker alleles involving at least three slippage events. (B) Schematic of the data obtained for Patient 16, wherein at least three pre-neoplastic fields exist.

FIG. 28. (A) Example of data from Patient 19 at amplicons (marker loci) 78, 66F, 87F, and 83F showing a complex pattern of marker alleles involving at least four slippage events. (B) Schematic of the data obtained for Patient 16, wherein at least four pre-neoplastic fields exist.

FIG. 29. Example electropherograms showing polyguanine tract genotype variation between a spatially separated pair of ulcerative colitis (UC) colon biopsies in three individuals. For each polyguanine marker, the “consensus” genotype is that most commonly observed among biopsies from a single patient. Mutant genotypes are those that differ from the consensus with respect to the length of at least one allele. X-axis indicates product length (bp), Y-axis represents signal intensity. Allele lengths are indicated, with mutant alleles in red. Non-indicated peaks are an artifact of PCR amplification (“stutter”).

FIG. 30. Frequency of mutant polyguanine genotypes by disease status. An average of 4.6 histologically non-dysplastic biopsies were obtained from 8 individuals with UC and no histological evidence of cancer or high-grade dysplasia anywhere in the colon (UC Non-Progressors) and 11 with UC and at least one site with adenocarcinoma and/or high-grade dysplasia (UC Progressors). Biopsies were divided into epithelial and stromal fractions, and both fractions were genotyped at 28 polyguanine markers. For each individual, the number of mutant genotypes out of the total number of successful genotypes (top), and the percentage of mutant genotypes (bars) are reported. Genotyping was performed under fully blinded conditions.

FIGS. 31A-F: Clonal patches identified by polyguanine mapping. Longitudinally opened colectomy specimens from three individuals (A-B: individual 13; C-D: individual 21; E-F: individual 19) are diagrammed with small boxes representing individual biopsies (˜9 mm²), taken at evenly spaced intervals within an alphanumeric grid. The histological diagnosis of each biopsy in the colon is indicated at left: NEG, negative for dysplasia; IND, indefinite for dysplasia; LGD, low grade dysplasia; HGD, high grade dysplasia; CAN, cancer; ?, no data. The genotypes of biopsies for specific polyguanine markers are indicated at right. Outlined boxes represent biopsies used in the initial study. Empty boxes represent biopsies not genotyped, “X” indicates unsuccessful genotyping. Grey fields indicate biopsies with the consensus genotype for the marker, and different colors represent distinct mutant genotypes within an individual for each marker. Clustering of identical mutant genotypes in adjacent biopsies suggests large, clonally-derived patches. For color representations, see FIG. 3 of Salk et al. 2009 (Salk J J et al., Proc Natl Acad Sci USA (2009) November 19).

FIGS. 32A-B: Polyguanine mapping of a complete UC Progressor colon. A longitudinally opened colectomy specimen is diagramed with boxes representing evenly spaced biopsies measuring ˜9 mm² within an alphanumeric grid. The histological diagnosis of each biopsy is indicated at far right: NEG, negative for dysplasia; IND, indefinite dysplasia; HGD, high grade dysplasia; CAN, cancer; ?, No data. Biopsy genotypes for various polyguanine marker are indicated in separate grids. “X” indicates unsuccessful genotyping. Grey fields indicate biopsies with the consensus genotype for the marker, and different colors represent distinct mutant genotypes for each marker. Dots indicate biopsies where a mixture of consensus and mutant genotypes were observed, suggesting a mixed population of cells with different genotypes. The total number of mutant genotypes identified across all markers is reported for each biopsy (heat map). Large, clonally-derived patches identified by three markers were observed near the cancer site. Numerous smaller patches were detected throughout the non-dysplastic portions of the colon. For color representations, see FIG. 4 of Salk et al. 2009 (Salk J J et al., Proc Natl Acad Sci USA (2009) November 19).

FIG. 33: Model for how mutant genotypes become identifiable as a result of clonal expansion. A) As cells divide throughout life, they acquire unique somatic mutations at polyguanine tracts. However, because such mutations are rare and independent, for any given locus the majority of cells do not carry a mutation. Consequently, genotyping identifies only the dominant, non-mutated (or “consensus”) genotype in a biopsy, and the individual mutant genotypes carried by single cells or small subclones are not observed. B) If an individual cell marked by a mutant allele clonally proliferates to populate a relatively large area, a unique genotype can come to dominate the sampled population and mutant alleles become detectable by genotyping. For a color representation, see FIG. 5 of Salk et al. 2009 (Salk JJ et al., Proc Natl Acad Sci USA (2009) November 19).

FIGS. 34A-B: Primer sequences and marker location for polyguanine homopolymer loci and Bethesda panel microsatellite instability (MSI) markers. Capitalized bases represent a non-homologous 5′ “pigtail” sequence added to limit genotyping artifacts.

FIGS. 35A-B: Relationship between location of histologically negative biopsies with clonally expanded mutations and nearest region of advanced histological disease. (A) The cumulative percentage of biopsies (unbroken line) and mutant genotypes (dashed line) are plotted as a function of increasing distance from nearest biopsy with cancer or HGD. The fraction of mutant genotypes identified tracks closely with the fraction of all biopsies analyzed suggesting that the probability of identifying a clonally expanded mutant genotype is independent of distance from dysplasia. (B) Raw data used to create the plot in 35A.

FIGS. 36A-G: UC progressors by prevalence of mutant genotypes detected and additional clinical parameters. For each panel, red bars indicate the percentage of genotypings within an individual differing from consensus and blue bars indicate an additional clinical feature: (A) highest grade histological diagnosis: high-grade dysplasia (HGD) or cancer; (B) presence or absence of concurrent primary sclerosing cholangitis (PSC); (C) gender; (D) age at time of sampling; (E) duration of clinically manifest ulcerative colitis; (F) symptomatic severity of ulcerative colitis. (G) Complete data for individuals.

FIGS. 37A-B: (A) Plot of prevalence of clonally expanded mutant genotypes as a function of microsatellite marker. Among marker sites with detectable mutations, a few were positive in 5-10% of biopsy genotypings although this was less than 4% for the majority. In approximately half of the polyguanine markers, and all four Bethesda panel microsatellite instability (MSI) markers tested, no mutant genotypes were identified. (B) Raw data used to generate the plot in 37A.

FIG. 38: Example electropherograms from a complete colon map showing consensus, mutant and mixed polyguanine tract genotypes. X-axis indicates product length (bp), Y-axis represents signal intensity. Allele lengths are indicated, with mutant alleles in red. Non-indicated peaks are an artifact of PCR amplification (“stutter”). For each polyguanine marker, the “consensus” genotype is that most commonly observed among biopsies from a single patient. Mutant genotypes are those that differ from the consensus with respect to the length of at least one allele. “Mixed” genotypes reflect a combination of consensus and mutant genotypes, and likely occur when a biopsy contains a mixture of cells with both consensus and a mutant genotype. Product length scale is listed at the top of each marker column. The alphanumeric code to the left of each electropherogram corresponds to the biopsy grid position in FIG. 4. Genotype calls are indicated to the right. Genotype assignments are based on the major peak(s) of each electropherogram. Adjacent “stutter” peaks are an artifactual consequence of PCR amplification.

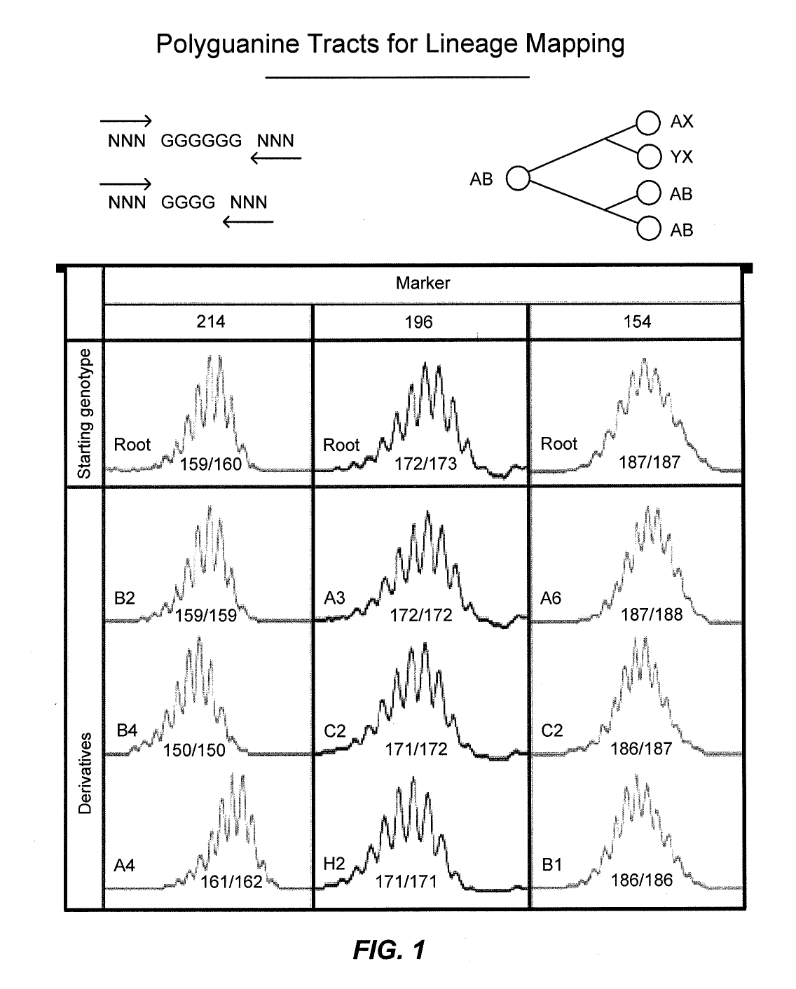

### DETAILED DESCRIPTION OF THE INVENTION

### A. Overview

Chronic inflammation predisposes to a variety of human cancers. Affected tissues slowly accumulate mutations, some of which affect growth regulation and drive successive waves of clonal evolution whereas a far greater number are functionally neutral and serve only to passively mark expanding clones. Ulcerative colitis (UC) is an inflammatory bowel disease in which up to 10% of patients eventually develop colon cancer. Studies are provided herein in which mutations in hypermutable intergenic and intronic polyguanine tracts have been mapped in patients with UC to delineate the extent of clonal expansions associated with carcinogenesis. Colon biopsies were genotyped for length altering mutations at 28 different polyguanine markers. In eight patients without neoplasia, only two mutations were detected in a single individual from among 37 total biopsies. In contrast, for eleven UC patients with neoplasia elsewhere in the colon, 63 mutations in 51 nondysplastic biopsies were identified, and every patient possessed at least one mutant clone. A subset of clones were large and extended over many square centimeters of colon. Of these, some occurred as isolated populations in non-dysplastic tissue, considerably distant from neoplastic lesions. Other large clones included regions of cancer, suggesting that the tumor arose within a pre-existing clonal field. These results demonstrate that neutral mutations in polyguanine tracts serve as a unique tool for identifying fields of clonal expansions, which may prove clinically useful for distinguishing a subset of UC patients who are at risk for developing cancer.

Cancer is a disease of somatic cellular evolution characterized by successive waves of mutation, selection and clonal expansion (Nowell PC, Science (1976) 194(4260):23-28; Loeb L A et al., Cancer Res (1974) 34(9):2311-2321; Merlo et al., Nat Rev Cancer (2006) 6(12):924-935). In many malignancies, most of this process is thought to occur within a relatively confined location, such as in the well-studied adenoma-to-carcinoma sequence of sporadic colorectal cancer (Fearon ER & Vogelstein B, Cell (1990) 61(5):759-767). However, cancers arising within the context of certain predisposing and preneoplastic conditions including oral leukoplakia (Brennan J A, et al., N Engl J Med (1995) 332(7):429-435), Barrett's esophagus (Prevo L J et al., Cancer Res (1999) 59(19):4784-4787), inflammatory bowel disease (Risques R A et al., Curr Opin Gastroenterol (2006) 22(4):382-390), and actinic keratosis (Kanjilal S, et al., Cancer Res (1995) 55(16):3604-3609), among others, appear to evolve more diffusely. The concept of “field effect”, first articulated by Slaughter (Slaughter DP et al., Cancer (1953) 6(5):963-968) more than half a century ago, describes the observation that cells within an area surrounding some tumor types display abnormal, yet not fully cancerous properties. More recently it has been appreciated that clonally-derived cell populations bearing a subset of the genetic and epigenetic abnormalities found in the tumor itself frequently form the basis for such fields (Braakhuis BJM et al., Cancer Res (2003) 63(8):1727-1730). Recognition that cancer-causing mutations may first emerge as widespread clones within non-neoplastic tissue has motivated efforts to identify the unique genetic changes that precede cancer for use in predicting its future development (Srivastava S, Gastrointestinal cancer research: GCR (2007) 1(4 Suppl 2):560-63).

Ulcerative Colitis (UC) is a chronic inflammatory disease of the colon that predisposes to colorectal cancer and affects approximately half a million individuals in the United States alone (Hanauer S B, Inflamm Bowel Dis (2006) 12 Suppl 1:S3-9). After 8 years of disease a patient's risk of cancer increases 0.5-1% per year, reaching nearly one in five after 30 years (Eaden JA et al., Gut (2001) 48(4):526-535). Longstanding UC presents a formidable clinical challenge; although cancer risk is markedly increased relative to an age-matched population, the absolute risk is not sufficiently high to justify the morbidity, cost and quality of life issues associated with prophylactic colectomy if management of symptoms is otherwise satisfactory. Because UC-derived dysplasias can be flat and hard to visualize endoscopically, current surveillance measures entail performing colonoscopy every 1-2 years to procure 30-60 biopsies for histological assessment in the hope that if cancer or advanced dysplasia exists, it will be found by random sampling (Ullman T et al., Inflamm Bowel Dis (2009) 15(4):630-638). This practice is expensive, insufficiently sensitive and only detects a neoplastic process once it has progressed to a morphologically recognizable stage.

It was previously demonstrated that genetic abnormalities common to UC-associated adenocarcinoma, including TP53 mutations (Brentnall TA, et al., Gastroenterology (1994) 107(2):369-378), ploidy abnormalities (Burmer G C, et al., Gastroenterology (1992) 103(5):1602-1610; Rubin C E, et al., Gastroenterology (1992) 103(5):1611-1620; Rabinovitch PS, et al., Cancer Res (1999) 59(20):5148-5153), and chromosomal losses and gains (Brentnall TA, et al., Gastroenterology (1994) 107(2):369-378); Chen R et al., Cancer Genet Cytogenet (2005) 162(2):99-106) can be found as large clonal fields in normal-appearing UC tissue outside of cancer sites. Some of these clonal lesions predict risk of future histological progression in individuals currently without dysplasia (Rubin C E, et al., Gastroenterology (1992) 103(5):1611-1620). A subset of individuals, however, progress in the absence of any of these markers. Recent cancer genome sequencing studies suggest that the genetic alterations responsible for driving tumorigenesis are highly diverse and unique to every tumor (Wood L D, et al. Science (2007) 318(5853):1108-1113; Fox E J et al., Cancer Res (2009) 69(12):4948-4950; Salk JJ et al., Ann Rev Path (2010) 5 (In press); each of which is hereby incorporated by reference in their entirety for all purposes). Although some genes are commonly mutated in specific cancers, others are mutated infrequently. Widespread clonal evolution could occur in nondysplastic colon prior to all UC-associated cancers, yet sometimes be undetectable by standard markers when clonal expansions are driven by mutation of unsuspected genes or regulators elsewhere within the (epi) genome.

One underpinning of certain aspects of the present invention was the hypothesis that the general phenotype of clonal expansion, rather than expansion of specific drivers, might serve as a more sensitive biomarker of prehistological neoplastic processes in UC. During normal mitosis, mutations occur at low frequency throughout the genome of all cells (Drake J W et al., Genetics (1998) 148(4):1667-1686), bestowing each cell with a unique fingerprint. While some mutations produce phenotypic changes, the vast majority occur outside of genes and regulatory regions and are likely to be functionally silent “passengers”. Irrespective of the specific mutation driving a clonal expansion, in theory, the progeny of any such event will be distinguishable from nearby cells by virtue of sharing the neutral mutational signature of the founding cell. The challenge to such an approach lies in the difficulty of locating these rare passenger mutations within a six gigabase genome. The present invention, is one aspect provides methods that solve these and other problems.

Short, repetitive sequences are replicated with significantly lower fidelity than other portions of the genome. Polyguanine tracts in particular, undergo insertion and deletion mutation with rates on the order of ˜10⁻⁴ per cell generation (Boyer J C, et al. Hum Mol Genet (2002) 11(6):707-713). These mutational hotspots serve as likely candidates for bearing lineage-identifying somatic variants. A high-throughput genomic approach to screen for mitotically acquired mutations at polyguanine sites was recently developed (Salipante S J & Horwitz M S, Proc Natl Acad Sci USA (2006) 103(14):5448-5453; Salipante S J et al., Genetics (2008) 178(2):967-977; each of which is hereby incorporated by reference in their entirety for all purposes) and have been used this to produce cell fate maps of mouse development (Salipante S J & Horwitz M S, Proc Natl Acad Sci USA (2006) 103(14):5448-5453; Salipante SJ, E M, & Horwitz M S (Phylogenetic Analysis of Developmental and Postnatal Mouse Lineages. (Submitted to Evolution and Development); each of which is hereby incorporated by reference in their entirety for all purposes). In the present study, we this technique was adapted to identify clonal expansions in UC colon. The results indicate that the method is highly effective at detecting discrete clones and that the presence of these clones in non-dysplastic tissue provides an almost complete ability to distinguish patients who have progressed to advanced histological disease from those who have not. It is demonstrated herein that the cell lineage information encoded in the genome by neutral mutant markers provides a novel tool for studying histologically invisible neoplastic processes and a potentially powerful method of identifying patients at greatest risk for developing cancer.

Accordingly, in one aspect, the present invention relates to the discovery of biomarkers and methods that allow for the detection of pre-cancerous fields. In one embodiment, the methods provided herein rely on high-throughput genotyping of many phenotypically neutral, yet somatically polymorphic (i.e., varies from one cell to another) polynucleotide sites, such as polyguanine tracts, in the genome to detect clonal expansion events that reflect the emergence of these fields. In blinded preliminary studies using tissue from 21 UC patients, 13 with cancer and 8 without, the subset with cancer were identified with 100% sensitivity and 94% specificity using microscopically normal biopsies from as much as 80 cm away from the cancer site. Although this ability alone will be of significant clinical utility, the biomarkers and methods provided herein are useful for detecting a process that predates cancer and thus allows for the identification of subjects who are most likely to progress to cancer months or years in advance in a wide range of cancers and with a wide range of predispositions or risk factors.

In one aspect, the present invention provides biomarkers for the detection of mutations that are clonally expanded to at least the size of a biopsy being removed. Generally, in order to see clonally expanded mutations there must be: 1) random mutations present in single cells and 2) subsequent clonal expansion of a subset of these cells. Large clonal expansions in the human colon are abnormal. The presence of random mutations in single cells, however, is normal. The frequency of mutations in a population of cells at any given time is a function of: A) the per-division mutation rate of long-lived stem cells and B) the number of cell divisions that have occurred to generate this population. If the per-cell division mutation rate is very high, even with a relatively low frequency of clonal expansions, there will be a good chance of detecting clonally expanded mutations because the probability of having at least one random mutation hitchhike along with this expansion will be high. Conversely, if the per-cell division mutation rate is low (for example, the same as in normal, non-UC colon), yet the number of cell divisions is high, the overall frequency of mutations will sufficiently high to have a good chance of one or more being carried along with a clonal expansion event and being detected. If the frequency of clonal expansions is very high, not only will this increase the overall frequency of mutations in the population by virtue of increasing the number of cell divisions (and therefore the number of mutations generated), there will be many more opportunities for a given random mutation to be clonally expanded and detected. In this fashion, the present invention provides methods of detecting clinically relevant and previously invisible prehistologic neoplasia (e.g., large clonally derived patches reflecting abnormal patterns of cell growth) through the use of neutral cell lineage markers.

### B. Biomarkers

In one aspect, the present invention provides biomarkers for the detection of a precancerous field in a subject. In some embodiments, these biomarkers comprise somatic genetic alterations that mark a clonal expansion event. In certain embodiments, these genetic alterations may comprise nucleotide mutations, including both point mutations and multibase mutations, insertions, deletions, duplications, translocations, inversions, loss of heterozygosity, gain of heterozygosity, a change in the size or number of nucleotide repeat tracts, and the like. Generally, the genetic alterations that serve as biomarkers for the invention, comprise alterations that are unlikely to cause or drive cancer progression. As such, these alterations may comprise a non-coding genetic alteration, for example an extragenic genomic mutation or intronic mutation. Similarly, an alteration that serves as a biomarker of the invention may comprise an intragenic silent or neutral alteration.

In some embodiments, a biomarker of the invention may comprise a genetic alteration that does not contribute to the progression of cancer in a subject. In one embodiment, a biomarker of the invention may comprise a genetic alteration that marks a clonal expansion event, but does not contribute to the progression of cancer or does not bear a functional consequence. In yet another embodiment, a biomarker of the invention may comprise an epigenetic change that marks a clonal expansion event, but that does not contribute to the progression of cancer. Non-limiting examples of epigenetic alterations include, changes in the methylation state of a genomic sequence or region, changes in the post-translational modification state of a nucleosome or region of chromatin, topological changes in the level of chromatin compaction at a gene or genomic loci, and the like.

In a particular embodiment, the present invention provides biomarkers for the detection of a precancerous field in a subject which comprise a genomic loci with a nucleotide repeat tract. In certain embodiments, a nucleotide repeat tract may be a mononucleotide repeat, wherein the repeated unit is a single nucleotide, for example, a Poly-A, Poly-T, Poly-G, or Poly-C tract. In other embodiments, a nucleotide repeat tract may be a dinucleotide repeat, wherein the repeated unit is two nucleotides, for example, a Poly-AT, Poly-AG, Poly-AC, Poly-TG, Poly-TC, or Poly-GC tract. In another embodiment, a nucleotide repeat tract may be a trinucleotide repeat, wherein the repeated unit is three nucleotides, for example, a Poly-ATA, Poly-ATT, Poly-ATC, Poly-ATG, Poly-AGA, Poly-AGT, Poly-AGC, Poly-AGG, and the like (i.e., any three nucleotide repeat sequence). In yet other embodiments, a nucleotide repeat tract may be a higher order nucleotide repeat, for example a tract comprising a repeating unit of at least about 4, 5, 6, 7, 8, 9, 10, 11, 12, 13, 14, 15, 16, 17, 18, 19, 20, 25, 30, 35, 40, 45, 50, 60, 70, 80, 90, 100, or more nucleotides. It will be understood by one skilled in the art that depending upon the nucleotide strand being detected (i.e. Watson or Crick strand), that complementary repeat sequences will be equivalent. For example, a Poly-G repeat is equivalent to a Poly-C, a Poly-A repeat is equivalent to a Poly-T repeat, a Poly-AT repeat is equivalent to a Poly-TA repeat, a Poly-GTA repeat is equivalent to a Poly-TAC repeat, and the like.

In certain embodiments of the invention, a nucleotide repeat tract may comprise at least about 3 repeated units. In other embodiments, a nucleotide repeat tract may comprise at least about 4, 5, 6, 7, 8, 9, 10, 11, 12, 13, 14, 15, 16, 17, 18, 19, 20, 25, 30, 35, 40, 45, 50, 60, 70, 80, 90, 100, or more repeated units.

In certain embodiments, a biomarker of the invention may comprise a Poly-G, Poly-C, Poly-A, or Poly-T nucleotide repeat tract. In a particular embodiment, the biomarker is a Poly-G or Poly-C repeat loci. In a specific embodiment, the loci is selected from the mononucleotide repeat marker loci found in Table 1. In a related embodiment, the biomarker is selected from those amplified in an amplicon selected from 66F, 87F, 41F, 18F, 21F, 103F, 83F, 107F, 104F, 52F, 32F, 26F, 58, 102F, 27F, 78, 47, 30F, 45F, 54F, 34F, 46F, 2F, 25F, 26F, 64, 81F, and 103F.

In a particular embodiment, a biomarker of the invention may comprise a nucleotide repeat tract in which a discreet change in the size of the tract is indicative of a clonal expansion event. Generally, a discreet change in the size of a nucleotide tract is relative to the size of the same nucleotide tract in a cell from the individual which has not undergone a clonal expansion event. For example, a discreet change in the size of a nucleotide tract in a first biopsy from a subject as compared to the size of the nucleotide tract in a second biopsy from the subject, or as compared to the size of the nucleotide tract in a biological sample taken from a location distal to the first biopsy. In certain embodiments, a change in the size of a nucleotide repeat tract arises as a result of a polymerase slippage event.

It will be understood by the skilled artisan that markers provided herein may be used singly or in combination with other markers for any of the uses, e.g., diagnosis, prognosis, or identification of a precancerous field or lesion.

### C. Primers and Probes

In another aspect, the present invention provides primers for the amplification of a biomarker provided herein. Generally, an amplification primer will be about 10 to about 100 nucleotides in length, for example, about 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, or more nucleotides in length. Typically, primers used for amplification of the biomarkers provided herein will hybridize to a genomic sequence located within about 1 kb of the marker loci. In certain embodiments, the primers provided will hybridize to a genomic sequence located within about 5 nt, or within about 10, 20, 30, 40, 50, 60, 70, 80, 90, 100, 125, 150, 175, 200, 250, 300, 350, 400, 500, 600, 700, 800, 900, 1000, 1100, 1200, 1300, 1400, 1500, 1600, 1700, 1800, 1900, 2000 or more nucleotides of the marker loci. In other embodiments, the primers may hybridize to a sequence located directly upstream or downstream of the marker loci, for example at the nucleotide that is directly adjacent to the marker loci at the 5′ or 3′ end of the loci. In yet other embodiments, a primer provided herein may partially or completely hybridize to a genomic sequence located within the marker loci, for example, a primer may be located externally, internally, or traverse a boundary between the marker loci and the flanking genomic sequences.

In another aspect, the present invention provides probes that hybridize to a biomarker provided herein. In certain embodiments, the probes provided comprise nucleic acid probes or modified nucleic acid probes, for example a polynucleotide comprising a locked nucleic acid (LNA). In some embodiments, the probes of the invention are useful for determining the sequence or length of a biomarker or marker loci of the invention. Generally, the probes of the invention are complementary or substantially complementary to the nucleic acid biomarkers provided herein. Typically, the probes of the invention hybridize to nucleic acid biomarkers under conditions of high stringency. A hybridization probe of the invention may be at least about 5 nucleotides in length, for example, about 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, or more nucleotides in length. Suitable formats for using the probes of the invention include, without limitation, solid-state surfaces, microarrays, slides, beads, qPCR probes, RT-PCR probes, and the like.

### D. Methods

In another aspect, methods of detecting the biomarkers of the invention are provided. In some embodiments, the methods comprise detecting a somatic genetic alteration by a method of nucleic acid sequencing, hybridization, or mass spectrometry. In a certain embodiment, the method comprises detecting a discrete change in the size of a genomic nucleotide repeat tract by a method of nucleic acid sequencing, hybridization, or mass spectrometry. Non-limiting examples of methods suitable for the detection of the biomarkers of the invention include PCR, qPCR, pyrosequencing, sequencing by ligation, primer extension, hybridization, mass spectrometry, capillary electrophoresis, nanopore transit, micro-cantilever disturbance, sequencing by synthesis, single-molecule sequencing, and single-molecule real-time sequencing.

In another aspect, the present invention provides methods of identifying the presence of a precancerous field in a subject. In one embodiment, the method comprises detecting a somatic genetic alteration in a biological sample from the subject. In certain embodiments, the method comprises detecting a discrete change in the size of a genomic mononucleotide repeat tract in a biological sample from the subject. In a specific embodiment, the method comprises detecting a discrete change in the size of a genomic Poly-G or Poly-C tract in a biological sample from the subject.

In one embodiment, the present invention provides a method for identifying the presence of a precancerous field in a subject, the method comprising (a) contacting a biological sample (i.e., a biopsy) from the subject with a detection reagent under conditions suitable to transform the detection reagent into a complex comprising the detection reagent and a nucleotide repeat tract biomarker provided by the present invention (for example, a Poly-G tract), (b) determining the length of the nucleotide repeat tract, and (c) identifying the presence of a precancerous filed in the subject if a there is a discrete change in the size of the nucleotide repeat tract. In one embodiment, the nucleotide repeat tract biomarker is selected from those found in Table 1. In other embodiments, the nucleotide repeat tract biomarker is at least about 2, 3, 4, 5, 6, 7, 8, 9, 10, 15, 20, 25, or more of the Poly-G tracts found in Table 1. In a related embodiment, the nucleotide repeat tract biomarker is a Poly-G tract selected from those amplified in amplicons 66F, 87F, 41F, 18F, 21F, 103F, 83F, 107F, 104F, 52F, 32F, 26F, 58, 102F, 27F, 78, 47, 30F, 45F, 54F, 34F, 46F, 2F, 25F, 26F, 64, 81F, and 103F.

In one embodiment, the present invention provides methods of identifying the presence of a precancerous field in a subject diagnosed with a disease or condition that is associated with an increased risk of developing cancer. In a related embodiment, methods are provided for identifying the presence of a precancerous field in a subject exposed to a carcinogen or agent that has been associated with an increased risk of developing cancer. In a specific embodiment, the methods comprise detecting a discrete change in the size of a genomic Poly-G tract in a biological sample from the subject. In one particular embodiment, the method comprises detecting at least one Poly-G tract selected from those found in Table 1. In other embodiments, the methods comprise detecting at least about 2, 3, 4, 5, 6, 7, 8, 9, 10, 15, 20, 25, or more of the Poly-G tracts found in Table 1. In a related embodiment, the methods comprise detecting at least one Poly-G tract selected from those amplified in amplicons 66F, 87F, 41F, 18F, 21F, 103F, 83F, 107F, 104F, 52F, 32F, 26F, 58, 102F, 27F, 78, 47, 30F, 45F, 54F, 34F, 46F, 2F, 25F, 26F, 64, 81F, and 103F. In one embodiment, the method comprises detecting all of the Poly-G tracts amplified in the above amplicons.

Many diseases and conditions have been found to be associated with an increased risk of developing cancer. Non-limiting example of such diseases and conditions include ulcerative colitis, Crohn's disease, Barrett's esophagus, gastritis, hepatitis, pancreatitis, oral leukoplakia, myelodysplastic syndromes, adenomatous polyps, diabetes, cholangitis, primary biliary cholangitis, primary sclerosing cholangitis, post-menopausal bleeding, and the like. In certain embodiments, the disease or condition associated with an increased risk of developing cancer may be a chronic inflammatory disease or condition or a gastrointestinal disorder.

Many carcinogenic agents have been found which are associated with an increased risk of developing cancer including, without limitation, asbestos, tobacco smoke, ultraviolet radiation, arsenic compounds (e.g. smelting byproducts, alloys, pesticides, herbicides, etc.), benzene, vinyl chloride, radioactive decay, and the like.

In one particular embodiment, the invention provides methods of identifying a precancerous field in a subject diagnosed with an inflammatory bowel disease. In one embodiment, the method comprises detecting a somatic genetic alteration that marks a clonal expansion event in a biological sample from the subject. In another embodiment, the method comprises detecting a discreet change in the size of a repeated genomic tract in a biological sample from the subject. In a specific embodiment, the method comprises detecting a discrete change in the size of a genomic Poly-G tract in a biological sample from the subject. In certain embodiments, the biological sample comprises a biopsy taken from the bowel of a subject diagnosed with ulcerative colitis or Crohn's disease. In other specific embodiments, the method comprises detecting at least one Poly-G marker locus selected from those found in Table 1 or in the list of amplicons found above.

In a specific embodiment, the method comprises isolating genomic DNA from a biopsy taken from the bowel of the subject, determining the length of a Poly-G tract in the biopsy, and comparing the length of the Poly-G tract to a reference, wherein a difference in the length of the Poly-G tract detected in the biopsy as compared to the reference indicates the presence of a precancerous field in the bowel of the subject. In certain embodiments, the presence of a pre-cancerous field indicates that the subject has an increased likelihood of developing cancer. In a particular embodiment, the cancer is colorectal or colon cancer.

In some embodiments, the methods provided herein comprise detection of a biomarker in a subset of cells from biological sample, for example, a biopsy, after prior enrichment for the assay. Methods of enrichment may include but are not limited to microdissection, EDTA epithelial shake-off, fluorescence-activated cell sorting (FACS), affinity chromatography, magnetic bead isolation, Transwell migration, density-gradient centrifugation, sedimentation rate centrifugation, or any other method known in the art.

In some embodiments, the methods comprise the detection of more than one Poly-G tract, wherein a difference in the length of any Poly-G tract indicates the presence of a precancerous field. In certain embodiments, the methods comprise detecting one or more Poly-G tracts from at least two biopsies taken from the bowel of the subject. In certain embodiments, at least about 2, 3, 4, 5, 6, 7, 8, 9, 10, 15, 20, 25, 30, 40, 50, 60, 70, 80, 90, 100, or more biopsies from the subject are used. In yet other embodiments, the methods comprise the use of 10 or fewer biopsies. In one particular embodiment, the method comprises the use of about 5 biopsies.

In certain embodiments, a reference may refer to a second sample taken from the subject, for example a second biological sample taken from a location proximal or distal to the first biological sample. A reference may also refer to a baseline or standard value as previously determined for the subject. In certain embodiments, the methods provided herein may comprise detecting a difference in the status of a biomarker at a single location in the subject (e.g., bowel, lung, liver, colon, etc.) over the course of time, for example over the course of days, months, years, or decades. In other embodiments, the methods provided herein may detect a difference in the status of a biomarker at one location as compared to a second location, for example in one biopsy as compared to a second biopsy, wherein the biopsies are taken at the same time or at substantially the same time. In yet other embodiments, a reference may refer to the status of a biomarker or marker allele from a normal individual or in the population as a whole.

In one embodiment, the invention provides methods of identifying a precancerous field associated with an increased risk of developing lung or oropharyngeal cancer in a subject chronically exposed to tobacco smoke. In certain embodiment, the biological sample may comprise cells isolated from sputum, bronchiolar lavage, or throat swabs.

In one embodiment, the invention provides methods of identifying a precancerous field associated with an increased risk of developing biliary tract cancer in a subject diagnosed with primary biliary cholangitis or primary sclerosing cholangitis. In certain embodiment, the biological sample may comprise cells isolated from bile samples.

In one embodiment, the invention provides methods of identifying a precancerous field associated with an increased risk of developing uterine cancer in a subject with post-menopausal bleeding. In one embodiment, the biological sample comprises an endometrial biopsy.

In one embodiment, the invention provides methods of identifying a precancerous field associated with an increased risk of developing bladder cancer in a subject who is chronically exposed to toxic chemicals, for example, smokers, metal workers, painters, hairdressers who use hair dye, rubber industry workers, leather workers, textile and electrical workers, miners, cement workers, transport operators, excavating-machine operators, and jobs that involve manufacture of carpets, paints, plastics, industrial chemicals, exposure to diesel exhaust, or diagnosed with a parasitic infection, for example, schistosomiasis. In one embodiment, the biological sample comprises cells isolated from a urine sample from the subject.

In another aspect, the present invention provides methods of providing a prognosis for a subject with an increased risk of developing cancer. In certain embodiments, the methods are for providing a prognosis for a subject diagnosed with a disease associated with an increased risk of developing cancer. In other embodiments, the methods are for providing a prognosis for a subject who has been chronically exposed to a known carcinogen. In one embodiment the method comprises isolating genomic DNA from a biological sample from the subject, detecting a biomarker in the biological sample, and comparing the status of the biomarker to a reference, wherein a difference in the status of the biomarker in the biological sample as compared to the reference indicates that the subject has an increased risk of developing cancer, thereby providing a prognosis for the subject.

In a specific embodiment, the method is for providing a prognosis for a subject diagnosed with a chronic inflammatory bowel disease, the method comprising the steps of isolating genomic DNA from a plurality of biopsies from the subject, determining the length of a poly-G tract in the biopsies, and comparing the lengths of the poly-G tract between the different biopsies, wherein a difference in the length of a poly-G tract between two biopsies indicates that the subject has an increased risk of developing bowel cancer, thereby providing a prognosis for the subject. In certain embodiments, the inflammatory bowel disease is ulcerative colitis or Crohn's disease and the cancer is colorectal or colon cancer. In some embodiments, the Poly-G tract is selected from those found in Table 1. In other embodiments, a plurality of Poly-G tracts are detected. In certain embodiments, the method comprises the use of 10 or fewer biopsies, for example about 1, 2, 3, 4, 5, 6, 7, 8, 9, or 10 biopsies from the subject. In one embodiment, the method comprises the use of about 5 biopsies.

In other embodiments, the methods provided herein are useful for providing a prognosis for a subject diagnosed with a disease or condition selected from ulcerative colitis, Crohn's disease, Barrett's esophagus, gastritis, hepatitis, pancreatitis, oral leukoplakia, myelodysplastic syndromes, adenomatous polyps, diabetes, cholangitis, primary biliary cholangitis, primary sclerosing cholangitis, post-menopausal bleeding, and the like, wherein a difference in the status of a biomarker between two biopsies indicates that the subject has a further increased risk of developing a cancer associated with the disease or condition.

In another aspect, the invention provides methods of assigning a course of action for the management or surveillance of a disease in a subject in need thereof. In certain embodiments, the methods comprise determining the status of a biomarker in a biological sample from the individual, determining if there is a difference in the status of the biomarker in the biological sample as compared to a reference, and assigning a course of action comprising further assessment or treatment if a difference exists. In certain embodiments, the biomarker comprises a nucleotide repeat tract and determining the status of the tract comprises determining the length of the repeat.

In one specific embodiment, the invention provides a method of assigning a course of treatment to a subject diagnosed with a chronic inflammatory bowel disease, the method comprising the steps of determining the length of a poly-G tract in a plurality of biopsies from the bowel of the subject, detecting a difference in the length of a poly-G tract in a first biopsy from the subject as compared to a second biopsy from the subject and assigning a course of treatment. In certain embodiments, the course of treatment is further diagnostic evaluation comprising a colonoscopy with biopsy. In other embodiments, the course of treatment comprises bowel surgery. In certain embodiments, the bowel surgery is bowel resection.

In one embodiment, the invention provides for a method of monitoring a subject diagnosed with an inflammatory bowel disease, the method comprising periodically removing a plurality of biopsies from the bowel of the subject, determining the length of at least one Poly-G tract in the plurality of biopsies, and determining if there is a discreet change in the length of a Poly-G tract in any of the biopsies, wherein an altered Poly-G tract indicates that a precancerous field or lesion has developed in the bowel of the subject. In certain embodiments, the method may additionally comprise the steps of performing a colonoscopy to further characterize the precancerous filed or lesion. In yet other embodiments, the method may further comprise assigning a course of treatment involving surgical resection of the bowel. In some embodiments, wherein the subject has been diagnosed with ulcerative colitis, the bowel resection may comprise large bowel or colon resection. In other embodiments, wherein the subject has been diagnosed with Crohn's disease, the bowel resection may comprise small bowel resection. Generally, the location and extent of the bowel resection may be determined based on the location of the biopsy showing an altered Poly-G tract or by a colonoscopy performed after identifying a biopsy having an altered Poly-G tract.

In certain embodiments, periodically may refer to about once a week. In other embodiments, periodically may refer to about once every other week, about once every third week, about once a month, about once every two months, about once every three months, about once every 4, 5, 6, 7, 8, 9, 10, 11, or 12 months. In other embodiments, periodically may refer to about 1 time a year, or about 2, 3, 4, 5, 6, 7, 8, 9, 10, 15, 20, 25, or more times a year. In yet other embodiments, periodically may refer to about once a year, or about once every other year, every third year, every 4, 5, 6, 7, 8, 9, 10, or more years.

In another aspect, the present invention provides methods of determining the surgical margins for a procedure involving tissue resection. In one embodiment, the methods comprise determining the status of a biomarker associated with a clonal expansion event in a plurality of biopsies from a subject, comparing the status of the biomarkers to either a reference or to each other, identifying a subset of biopsies containing cells that have undergone a clonal expansion event, and correlating the location from which the biopsies were removed to surgical margins for a resection procedure.

The methods provided herein for determining a surgical margin are generally applicable to tissue resection surgeries involving the removal of precancerous or cancerous fields or tissues. In this fashion, an individual diagnosed with a disease or condition associated with an increased risk of developing cancer may be monitored for the establishment of a precancerous field or lesion using the methods provided herein. Upon detection of a precancerous filed or lesion, preventative measures, such as tissue resection, may be employed in order avoid cancer progression.

In one embodiment, a method is provided for determining the surgical margins for a bowel resection in a subject diagnosed with a chronic inflammatory bowel disease, the method comprising removing a plurality of biopsies from the bowel of the patient, determining the status of at least one Poly-G tract in the plurality of biopsies, identifying a subset of biopsies with a discreet difference in at least one Poly-G tract, and correlating the location from which the biopsies were removed to surgical margins for a bowel resection. In some embodiments, the method may further comprise the initial steps of identifying a first biopsy with an altered Poly-G tract, for example during a routine surveillance or check-up, and then removing a plurality of biopsies flanking or proximal to the first biopsy. In some embodiments, the methods may further comprise performing a colonoscopy, for example, to further characterize the extent of the precancerous field or lesion.

In another particular embodiment, the invention provides a method of determining a surgical margin for Mohs surgery, comprising determining the status of a biomarker associated with a clonal expansion event (e.g. a somatic genetic alteration or Poly-G tract) in a tissue section surgically removed during surgery, identifying the tissue sample as cancerous or precancerous, and determining a surgical margin for the removal of a tumor.

In another aspect, the present invention provides methods of assessing the adequacy of a surgical margin used for a tissue resection or tumor removal procedure. In certain embodiments, the method comprises determining the status of a biomarker associated with a clonal expansion event in a biological sample taken proximal to the site of a tissue or tumor resection, and determining if cells in the biological sample have undergone a clonal expansion event, thereby determining if the surgical margins for the procedure were adequate. In certain embodiments, wherein the surgical margins were not adequate, the method may further comprise the steps of determining new surgical margins for a follow-up procedure. The methods provided herein for assessing the adequacy of a surgical margin are generally applicable to any procedure involving the resection of a tumor or a precancerous field or lesion.

In one embodiment, a method is provided for assessing the adequacy of a surgical margin used for a bowel resection. In a particular embodiment, the method comprises the steps of removing a biopsy from a site proximal to a margin of the bowel resection, determining the length of a Poly-G tract in the biological sample, comparing the length of the Poly-G tract to a reference, and determining whether or not there is a discreet change in the length of the Poly-G tract, wherein an altered Poly-G tract indicates that the surgical margin used was not adequate. In certain embodiments, wherein the surgical margins were determined to be inadequate, the method further comprises determining new surgical margins for a follow-up procedure. In other embodiments, wherein the surgical margins were determined to be inadequate, the method further comprises performing a colonoscopy in order to further characterize the cancerous or precancerous field or lesion.

### E. Kits and Reagents

In another aspect, the invention provides kits for diagnosing or providing a prognosis for an increased risk of developing cancer in a subject. In some embodiments, the kits may comprise a primer or hybridization probe of the invention. In certain embodiments, the kits provided herein are useful for providing a prognosis of an increased risk of developing bowel cancer in a subject diagnosed with a chronic inflammatory disease, for example, ulcerative colitis or Crohn's disease. In other embodiments, kits of the invention may be useful for determining the margins of a tissue or tumor resection. For example, for determining the margins of a sub-colonic resection in a patient who has been identified as having a discrete change in the size of a genomic poly-G tract.

In another aspect, the invention provides solid-state platforms for diagnosing or providing a prognosis of an increased risk of developing cancer in an individual. In certain embodiments, the platform comprises a plurality of nucleic acids that hybridize to Poly-G tracts associated with a clonal expansion event. In one particular embodiment, the solid-state platform comprises a plurality of probes that hybridize to a plurality of loci found in Table 1. In certain embodiment, the solid-state platform may comprises a microarray, bead, or sequencing cassette.

Any known microarray and/or method of making and using microarrays can be used in the practice of the present invention, such as those disclosed, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston, Curr. Biol. 8:R171-R174, 1998; Schummer, Biotechniques 23:1087-1092, 1997; Kern, Biotechniques 23:120-124, 1997; Solinas-Toldo, Genes, Chromosomes & Cancer 20:399-407, 1997; Bowtell, Nature Genetics Supp. 21:25-32, 1999. See also published U.S. patent applications Ser. Nos. 20010018642; 20010019827; 20010016322; 20010014449; 20010014448; 20010012537; 20010008765.

### DEFINITIONS

As used herein, the terms “precancerous field”, “preneoplastic field”, and “precancerous lesion” interchangeably refer to a section of tissue consisting of cells with a common monoclonal origin that have histological or genetic abnormalities characteristic of dysplasia, but which do not display invasive or metastatic properties characteristic of cancer cells.

As used herein, the term “clonal expansion event” refers to the localized propagation of preneoplastic or dysplastic cells. In the context of the present disclosure, a clonal expansion event is precursory to the establishment of a precancerous field.

As used herein, the term “Progressor” refers to a subject who has developed a cancer after being diagnosed with an elevated risk of developing said cancer. For example, a “UC Progressor” refers to an individual diagnosed with ulcerative colitis, who develops colon cancer. Similarly, a “Crohn's Progressor” refers to an individual diagnosed with Crohn's disease, who develops intestinal cancer or small intestinal cancer. In certain instances a “Progressor” may refer to an individual who is exposed to a factor associated with an increased risk of developing a cancer and then develops said cancer. For example, a smoker who develops lung cancer or a subject exposed to asbestos who develops mesothelioma.

As used herein, the term “nucleotide repeat tract” refers to a stretch of nucleotides in the genome wherein a base unit is repeated. A nucleotide repeat tract may be single nucleotide, i.e. a mononucleotide repeat, wherein a single nucleotide, for example a guanine in a poly-G tract or cytosine in a poly-C tract, is repeated in the genome. In other embodiments, a nucleotide repeat tract may be a dinucleotide repeat wherein a two nucleotide sequence is repeated, for example AG in a poly-AG tract. In yet other embodiments, a nucleotide repeat tract may be a trinucleotide repeat tract, or a larger nucleotide repeat tract, for example one in which a sequence of at least about 4, 5, 6, 7, 8, 9, 10, 15, 20, 25, 30, 35, 40, 45, 50, 60, 70, 80, 90, 100 or more nucleotides are repeated.

As used herein, the “status” of a nucleic acid biomarker generally refers to whether or not the sequence has been altered. For example, the status of a marker locus may be wild type or altered (mutant), wherein the altered state is indicative or associated with a clonal expansion event, precancerous field, or increased risk of developing cancer. In a specific embodiment, the status of a Poly-G marker locus (biomarker) in a subject may wild type, wherein the length of the Poly-G tract is unaltered or the same as in a non-precancerous cell from the subject, or altered, wherein a discreet change in the length of the Poly-G tract has occurred. Generally, a biomarker with an altered status, for example, a discreet change in the length of a Poly-G tract, will be associated with a clonal expansion event, precancerous field, or a further increased risk of developing cancer.

As used herein, the term “inflammatory bowel disease” refers to a class of inflammatory conditions affecting the small and/or large intestine of a subject, such as a human. Exemplary inflammatory bowel diseases include, without limitation, ulcerative colitis (UC), Crohn's disease (CD), collagenous colitis, lymphocytic colitis, ischaemic colitis, diversion colitis, Behcet's syndrome, infective colitis, indeterminate colitis, celiac disease, sprue, tropical sprue, and the like.

As used herein, the tern “gastrointestinal disorder” refers to a class of diseases and conditions that affect the gastrointestinal tract of a subject, such as a human. For example, a gastrointestinal disorder may include a disease or condition of the mouth, pharynx, esophagus, stomach, duodenum, jejunum, ileum, ileo-cecal complex, large intestine, small intestine, cecum, colon, rectum and the like. In certain embodiments, a gastrointestinal disorder may refer to a disease or condition of an accessory organ to the gastrointestinal tract, such as the liver, gallbladder, pancreas, and the like. Exemplary gastrointestinal disorder include, but are not limited to, colorectal cancer, diverticulitis, gastroenteritis, giardiasis, inflammatory bowel disease (Crohn's disease and ulcerative colitis), irritable bowel syndrome (IBS), pancreatitis, cholera, peptic ulcer disease, Barrett's esophagus, gastritis, hepatitis, and the like.

As used herein, the term “cancer” refers to mammalian cancers and carcinomas, for example, human, murine, rodent, and the like, leukemias, sarcomas, adenocarcinomas, lymphomas, solid and lymphoid cancers, etc. Examples of different types of cancer include, but are not limited to, colon cancer, colorectal cancer, gastrointestinal cancer, esophageal squamous cell carcinoma or adenocarcinoma, gastric carcinoma, signet ring cell carcinoma, gastric lymphoma (MALT lymphoma), linitis plastica, duodenal cancer (e.g. adenocarcinoma), cancer of the appendix (e.g. carcinoid or pseudomyxoma peritonei), colon/rectum colorectal polyps, familial adenomatous polyposis, colonic adenocarcinoma, familial adenomatous polyposis, hereditary nonpolyposis colorectal cancer, anal cancer, upper or lower gastrointestinal stromal tumors, Krukenberg tumor, liver hepatocellular carcinoma (e.g. fibrolamellar), hepatoblastoma, hepatocellular adenoma, focal nodular hyperplasia, nodular regenerative hyperplasia, biliary tract neoplasm, cholangiocarcinoma, Klatskin tumor, gallbladder cancer, pancreas adenocarcinoma, pancreatic ductal carcinoma, pancreatoblastoma, primary peritoneal cancer, breast cancer, gastric cancer, bladder cancer, ovarian cancer, thyroid cancer, lung cancer, prostate cancer, uterine cancer, testicular cancer, neuroblastoma, squamous cell carcinoma of the head, neck, cervix and vagina, multiple myeloma, soft tissue and osteogenic sarcoma, liver cancer (i.e., hepatocarcinoma), renal cancer (i.e., renal cell carcinoma), pleural cancer, pancreatic cancer, cervical cancer, anal cancer, bile duct cancer, gastrointestinal carcinoid tumors, esophageal cancer, gall bladder cancer, small intestine cancer, cancer of the central nervous system, skin cancer, choriocarcinoma; osteogenic sarcoma, fibrosarcoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, and acute myelocytic leukemia, chronic myelocytic leukemia, promyelocytic leukemia, and the like.

As used herein, a “biological sample” includes sections of tissues such as biopsy and autopsy samples, and frozen sections taken for histologic purposes. Such samples also include, without limitation, blood and blood fractions or products (e.g., serum, plasma, platelets, red blood cells, and the like), sputum or saliva, lymph and tongue tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., colon, intestine, prostate, kidney, lung, breast, bladder, lymph node, liver, bone marrow, blood cell, etc.), the disease or condition being monitored (e.g., ulcerative colitis, Crohn's disease, hepatitis, pancreatitis, oral leukoplakia, myelodysplasia, etc.), the chronic exposure being monitored (e.g., tobacco smoke, radioactive decay, ultraviolet irradiation, etc.), among other factors. Representative biopsy techniques include, but are not limited to, excisional biopsy, incisional biopsy, needle biopsy, surgical biopsy, core-needle biopsy, fine-needle aspiration biopsy, bone marrow biopsy, epithelial brushings, epithelial swabings, epithelial scrapings, and the like. Biopsy techniques are discussed, e.g., in Kasper et al., Harrison's Principles of Internal Medicine, eds., 16^{th} ed., Chapter 70 and throughout Part V (2005).

As used herein, “providing a prognosis” refers to providing a prediction of the likelihood of developing cancer, progression of a disease into a cancer, predictions of cancer free and overall survival, the probable course and outcome of a tissue resection or preventive cancer therapy, or the likelihood of recovery from a precancerous field or lesion, in a subject.

As used herein, the term “diagnosis” refers to the identification of a precancerous state, such as the detection of a precancerous field or lesion, in a subject diagnosed with a disease associated with an increased risk of developing cancer or in a subject who has been chronically exposed to a carcinogen. The methods of diagnosis provided by the present invention can be combined with other methods of diagnosis well known in the art. Non-limiting examples of other methods of diagnosis include, colonoscopies, narrow band imaging, cytological screening, ductal lavage, pap smear, thermography, co-axial tomography (CAT) scans, positron emission tomography (PET), radionuclide scanning, biopsy, histologic methods, karyotypic methods, magnetic resonance imaging (MM), and the like.

As used herein, a “nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and/or which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, polynucleotide, and primer.

The terms “identical” or percent “identity,” in the context of two or more nucleic acids sequences, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 nucleotides in length, or over a region that is at least about 15, 20, 25, 30, 35, 40, 50, 60, 70, 80, 90, 100, 125, 150, 175, 200, 250, 300, 350, 400, 500, 600, 700, 800, 900, 1000, or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous nucleotide positions selected from the group consisting of from about 10 to 1000, or from about 20 to about 500, or from about 50 to about 250, or from about 100 to about 200, or a region of at least about 10, 15, 20, 25, 30, 35, 40, 50, 60, 70, 80, 90, 100, 125, 150, 175, 200, 250, 300, 350, 400, 500, 600, 700, 800, 900, 1000, or more nucleotides in length in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Nat'l. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithms that are suitable for determining percent sequence identity and sequence similarity for nucleic acid sequences are the BLAST and BLAST 2.0 algorithms, which are described in Altschul et al., Nuc. Acids Res., 25:3389-3402 (1977) and Altschul et al., J. Mol. Biol., 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

Hybridization conditions for nucleic acids in the methods of the present invention are well known in the art. Hybridization conditions may be high, moderate or low stringency conditions. Ideally, nucleic acids will hybridize only to complementary nucleic acids and will not hybridize to other non-complementary nucleic acids in the sample. The hybridization conditions can be varied to alter the degree of stringency in the hybridization and reduce background signals as is known in the art. For example, if the hybridization conditions are high stringency conditions, a nucleic acid will bind only to nucleic acid target sequences with a very high degree of complementarity. Low stringency hybridization conditions will allow for hybridization of sequences with some degree of sequence divergence. The hybridization conditions will vary depending on the biological sample, and the type and sequence of nucleic acids. One skilled in the art will know how to optimize the hybridization conditions to practice the methods of the present invention.

For PCR, a temperature of about 36 C is typical for low stringency amplification, although annealing temperatures may vary between about 32 C and 48 C depending on primer length. For high stringency PCR amplification, a temperature of about 62 C is typical, although high stringency annealing temperatures can range from about 50 C to about 65C, depending on the primer length and specificity. Typical cycle conditions for both high and low stringency amplifications include a denaturation phase of 90 C-95 C for 30 sec-2 min., an annealing phase lasting 30 sec.-2 min., and an extension phase of about 72 C for 1-2 min. Protocols and guidelines for low and high stringency amplification reactions are provided, e.g., in Innis et al. (1990) PCR Protocols, A Guide to Methods and Applications, Academic Press, Inc. N.Y.).

“Amplification” or an “amplification reaction” refers to any chemical reaction, including an enzymatic reaction, which results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691 (1992); Walker PCR Methods Appl 3(1):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), rolling circle amplification (RCA) (Lisby, Mol. Biotechnol. 12(1):75 (1999)); Hatch et al., Genet. Anal. 15(2):35 (1999)) and branched DNA signal amplification (bDNA) (see, e.g., Iqbal et al., Mol. Cell Probes 13(4):315 (1999)).

The reagents used in an amplification reaction can include, e.g., oligonucleotide primers; borate, phosphate, carbonate, barbital, Tris, etc. based buffers (see, U.S. Pat. No. 5,508,178); salts such as potassium or sodium chloride; magnesium; deoxynucleotide triphosphates (dNTPs); a nucleic acid polymerase such as Taq DNA polymerase; as well as DMSO; and stabilizing agents such as gelatin, bovine serum albumin, and non-ionic detergents (e.g. Tween-20).

A “label” or a “detectable moiety” is a composition detectable by spectroscopic, photochemical, biochemical, immunochemical, chemical, or other physical means. Labels useful for the detection of nucleic acid probes and primers of the invention include, without limitation, fluorescent dyes (e.g., Cy5, Cy3, FITC, rhodamine, lanthamide phosphors, Texas red, FAM, HEX, NED, TET, ROX, JOE, VIC, TAMRA), ³²P, ³⁵S, ³H, ¹⁴C, ¹²⁵I, ¹³¹I, electron-dense reagents (e.g., gold), enzymes, e.g., as commonly used in an ELISA (e.g., horseradish peroxidase, beta-galactosidase, luciferase, alkaline phosphatase), colorimetric labels (e.g., colloidal gold), magnetic labels (e.g., Dynabeads), biotin, dioxigenin, aptamers, or haptens and proteins for which antisera or monoclonal antibodies are available. The label can be directly incorporated into the nucleic acid to be detected, a dye that binds to a probe-target hybrid such as SYBR green or SYBR gold, or it can be attached to a probe (e.g., an oligonucleotide) or antibody that hybridizes or binds to the nucleic acid to be detected. The detectable label can be incorporated into, associated with or conjugated to a nucleic acid. The association between the nucleic acid and the detectable label can be covalent or non-covalent. Label can be attached by spacer arms of various lengths to reduce potential steric hindrance or impact on other useful or desired properties.

### Compositions, Kits, and Integrated Systems

The present invention also provides kits to facilitate and/or standardize the methods provided herein. Materials and reagents for executing the various methods of the invention can be provided in kits to facilitate these methods. As used herein, the term “kit” refers to a combination of articles that facilitate a process, assay, analysis, diagnosis, prognosis, or manipulation.

In one embodiment, the kits provided by the present invention may comprise a nucleic acid primer for the amplification of a genomic marker locus associated with a clonal expansion event. In certain embodiments, the kits may comprise a primer mix for the multiplex linear amplification of multiple genomic loci. In other embodiments, the kits of the invention may comprise a hybridization probe array for use in detecting somatic genetic alteration associated with clonal expansion events. In certain embodiments, the present invention provides kits useful for the identification of a precancerous field or lesion in a subject diagnosed with a disease associated with an increased risk of developing cancer.

The invention provides assay compositions for use in solid phase assays; such compositions can include, for example, one or more polynucleotides of the invention immobilized on a solid support, and a labeling reagent. In each case, the assay compositions can also include additional reagents that are desirable for hybridization.

Optical images viewed (and optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical images.

### EXAMPLES

### Example 1

Repetitive DNA is particularly prone to oxidative injury and slippage upon repair. More than a decade ago, it was observed that patients with UC had an elevated rate of microsatellite instability, even in non-dysplastic mucosa; however, using what later came to be known as the Bethesda panel, this microsatellite instability was lower than that seen in mismatch repair deficient, microsatellite instable cancers. With the tools available at the time, the sensitivity of this finding was low, and the observation retreated into obscurity. In more recent years, in vitro studies of oxidative injury to DNA suggested that polyguanine (poly-G) tracts are at least 2-3 times more sensitive to peroxide than other types of repeats tested. At the same time, new methods have emerged that allow for significantly more efficient analysis. In the present studies, the biomarker utility of detecting clonal expansion events marked by slippage events in polyguanine microsatellite tracts was assessed for using a highly automated, high throughput assay. This assay differs from microsatellite instability analysis in that it detects clonal expansion of a cell lineage bearing a discrete microsatellite allele that differs from the consensus majority of the organism or other parts if the organ being sampled rather than the presence of a highly heterogeneous mixture of alleles indicative of high-level microsatellite instability such as seen in traditionally-defined microsatellite instability such as found in microsatellite unstable (MSI) sporadic colorectal cancers bearing deficiencies in MSH or MLH gene expression. The assay requires only 4 ng of DNA per locus genotyped and PCR is carried out in a robotically loaded 384 well format. The PCR is followed by multiplex capillary electrophoresis on an ABI 3700 sequencer. Allele calls are assigned based on a combination of automatic ABI software, PERL scripts and expert visual verification.

The initial studies were performed on 13 patients: 3 non-UC, 6 Non-Progressors and 4 Progressors. A total of 16 poly-G tracts were amplified from colonic epithelial DNA and the allelotypes assessed. In brief, 0/6 Non-Progressors had poly-G slippage while 4/4 Progressors did have slippage. All of these studies were performed on non-dysplastic colonic biopsies. Next, steps were undertaken to assess the reproducibility of the assay, to optimize the performance of the assay and to determine whether it could be performed in formalin fixed paraffin-derived tissue as well as fresh frozen. To that end, a second pilot study was performed in a blinded fashion. The expanded group of patients included 3 non-UC patients, 8 UC Non-Progressors and 11 UC Progressors. Representative data from patients 1 (non-UC), 4 (UC Non-Progressor), and 11 (UC-Progressor), can be found in Tables 2, 3, and 4, respectively.

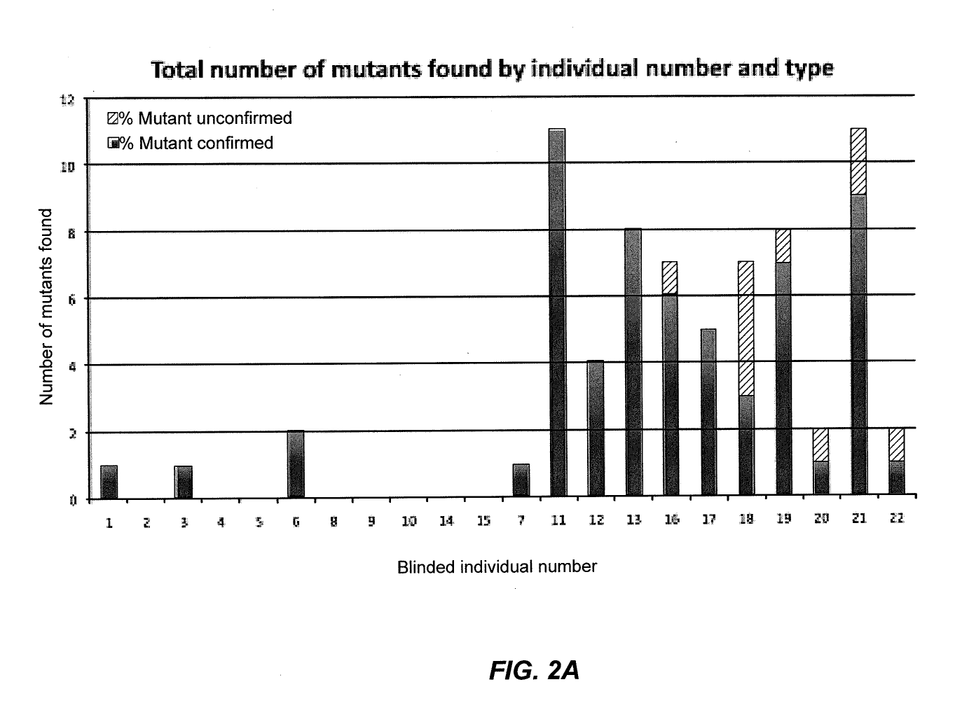

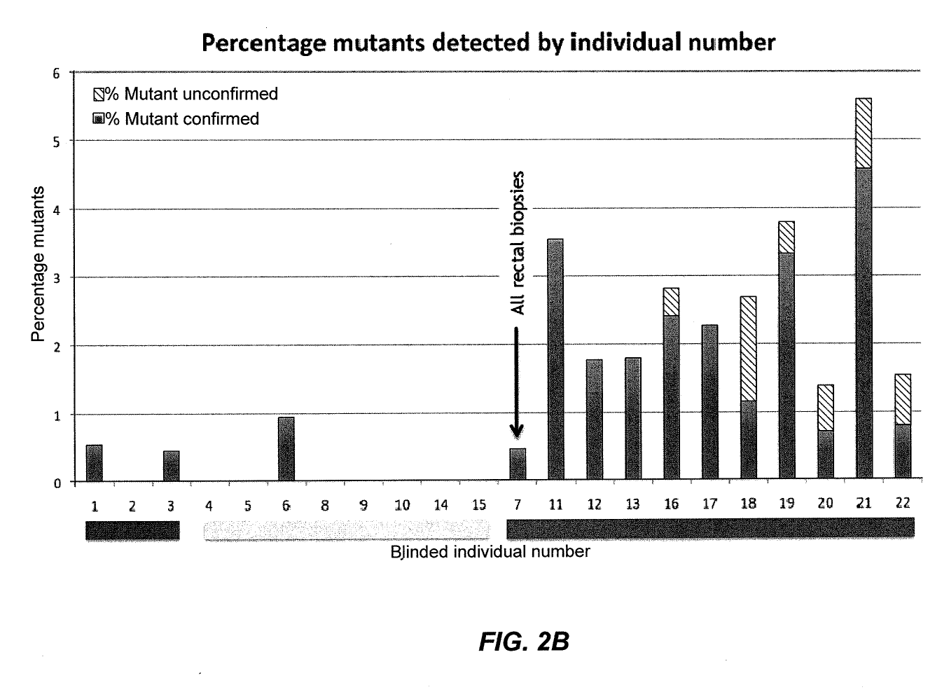

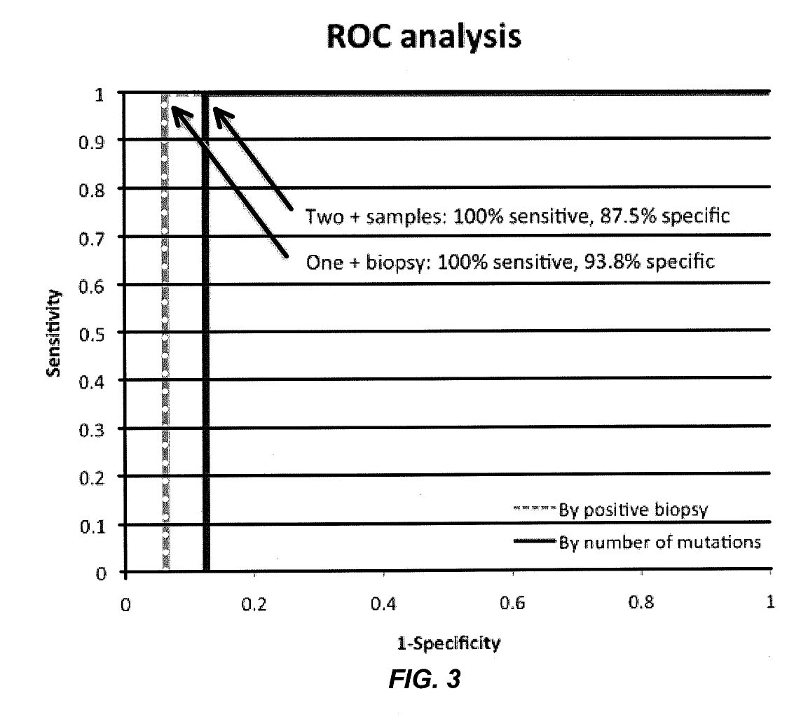

An average of 4.8 biopsies was tested per patient probing a total of 32 poly-G tracts, which led to a total of 5157 successful genotypes on 200 samples. In this study, the performance of the poly-G test had 100% sensitivity and 94% specificity (Fishers exact test with the two-tailed P value<0.0001, 95% CI 0.88-0.96 (FIG. 3)). The number of slippage events detected per individual is represented in FIG. 2. It is of note that the Progressor individuals with the fewest number of mutations either had an unusually low number of biopsies tested (cases 7 and 22) or an unusually high number of genotyping reactions that failed (case 22). Approximately 79% of the mutations were found in the epithelium, while about 21% were found in the stroma.

A list of Amplicons, primers, and genomic loci used in the present studies can be found in Table 1. Of note, all reverse primers incorporated a “Pigtail sequence” to prevent genotyping artifacts (Brownstein M J, Carpten J D, Smith J R: Modulation of non-templated nucleotide addition by Taq DNA polymerase: primer modifications that facilitate genotyping. Biotechniques 1996, 20(6):1004-1006, 1008-1010).

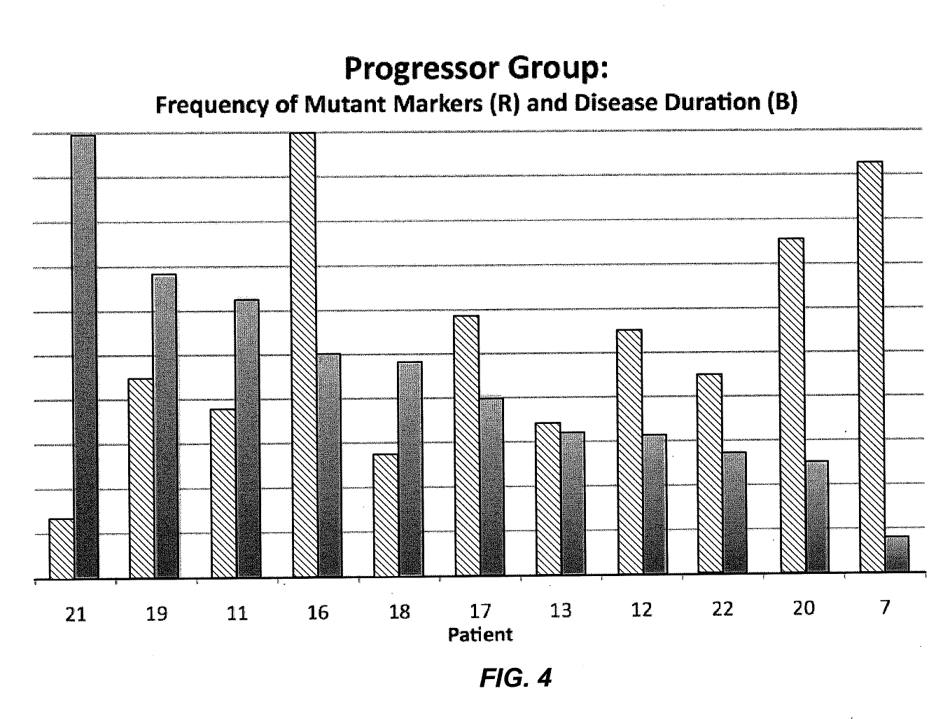

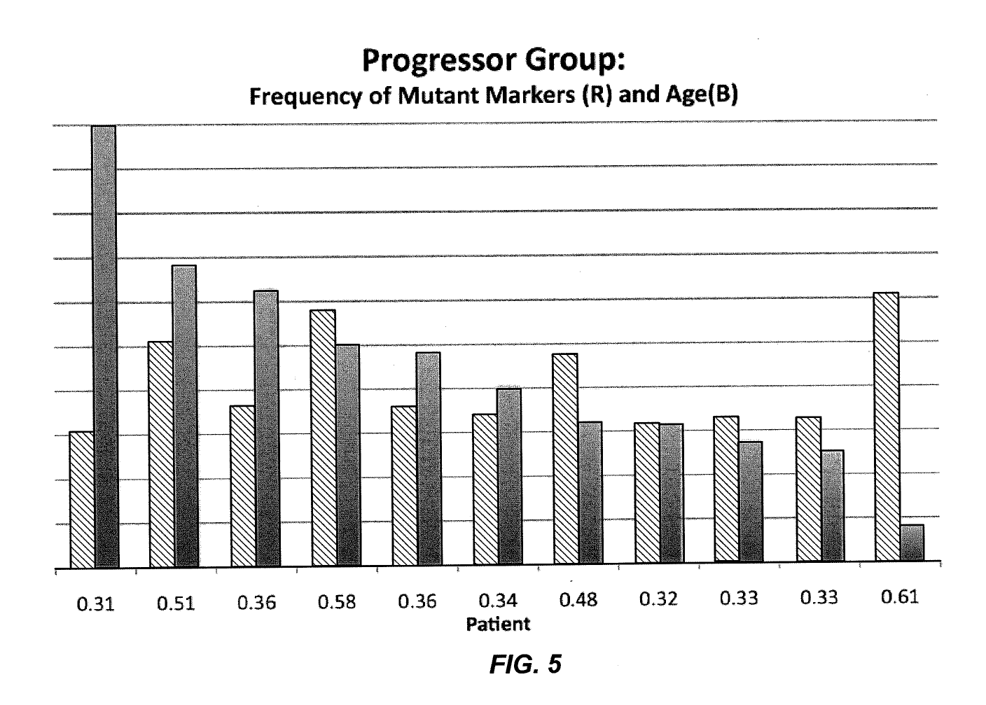

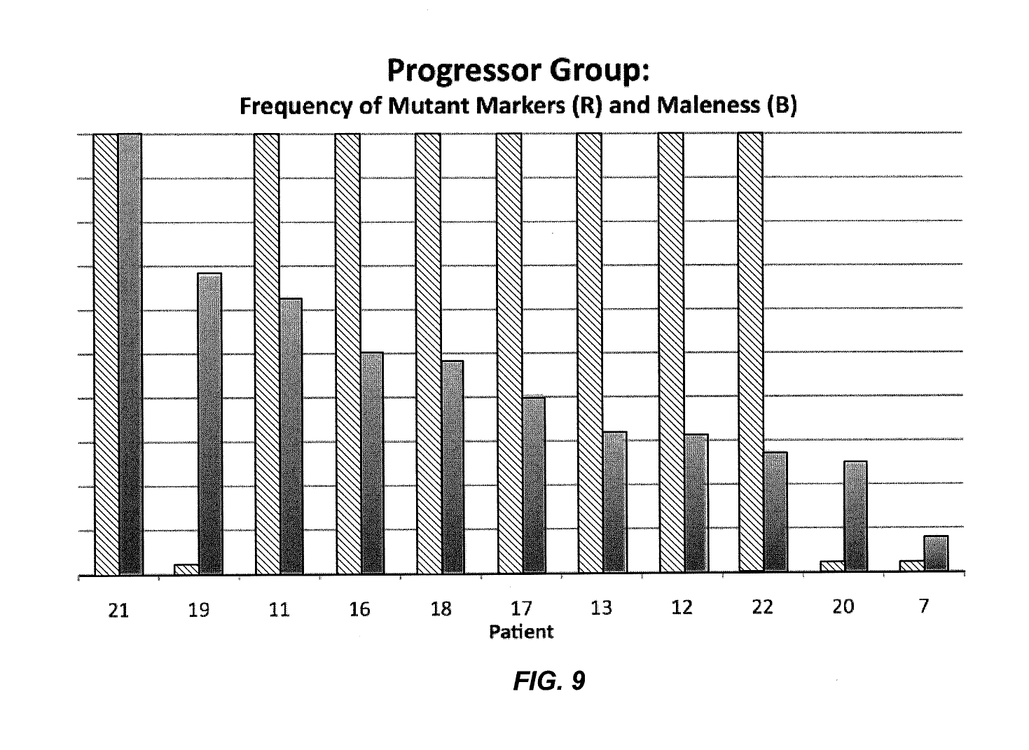

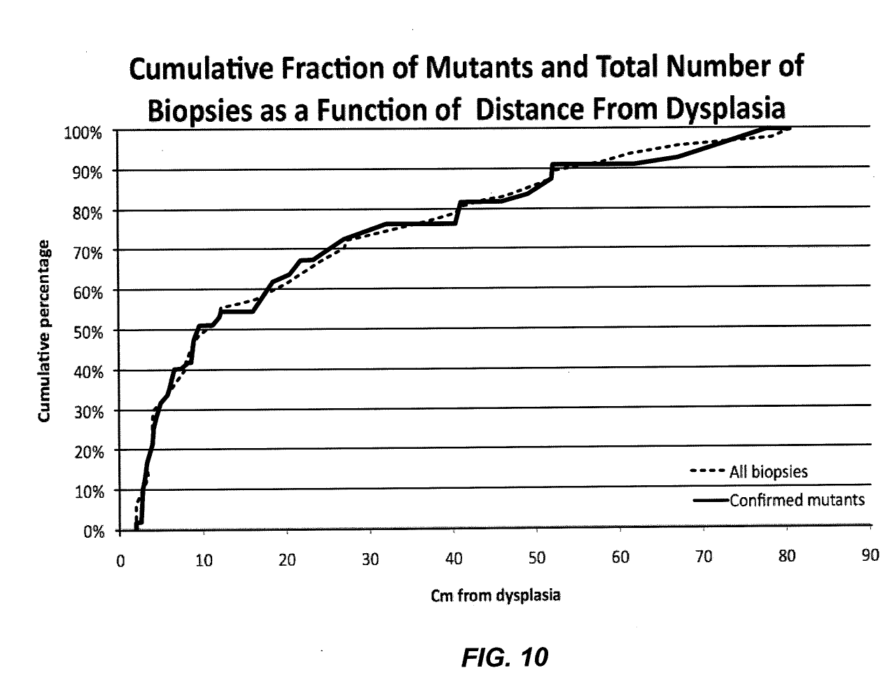

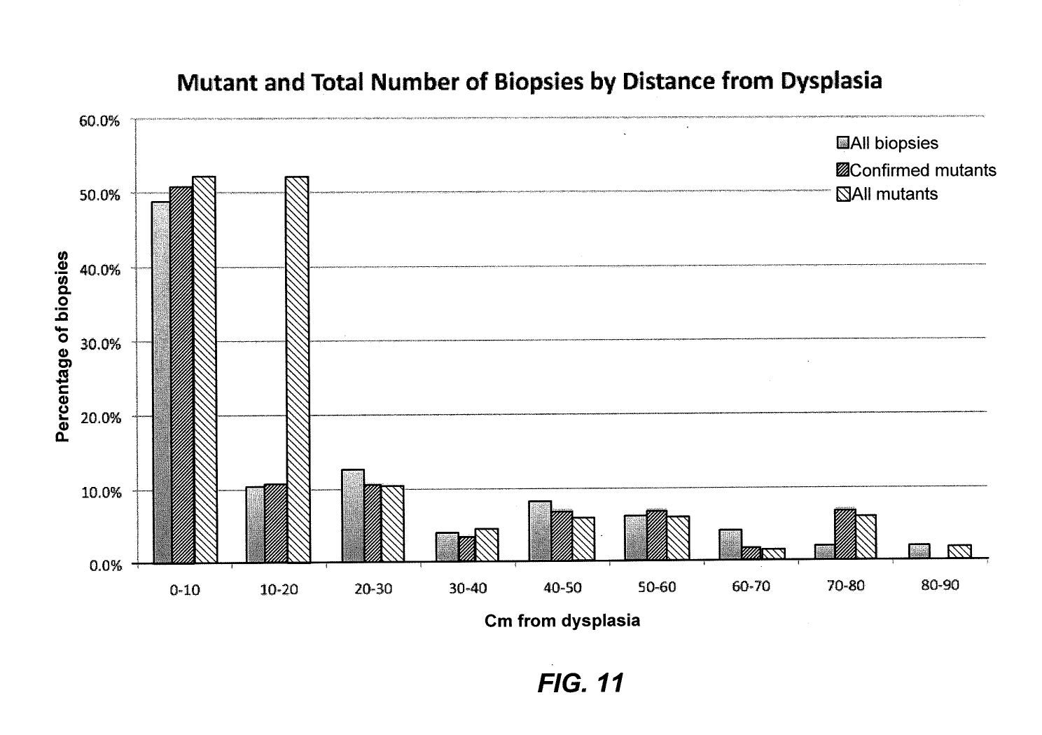

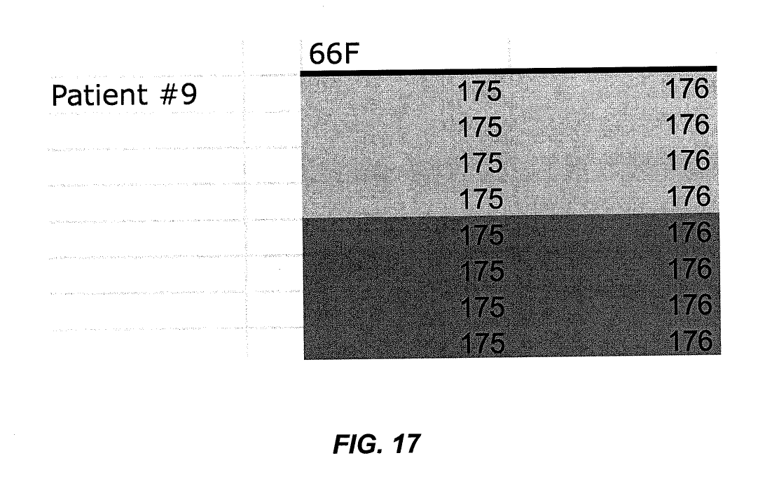

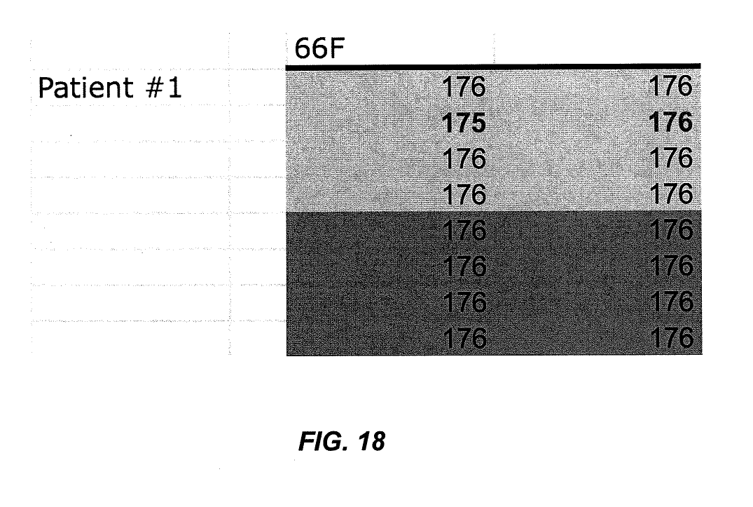

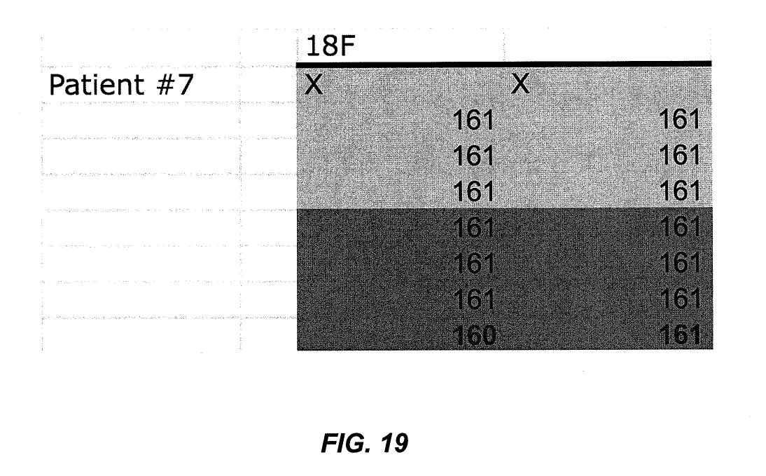

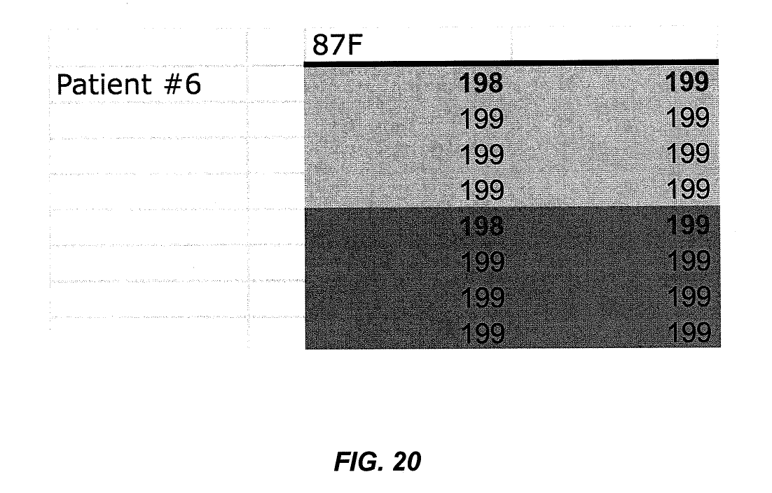

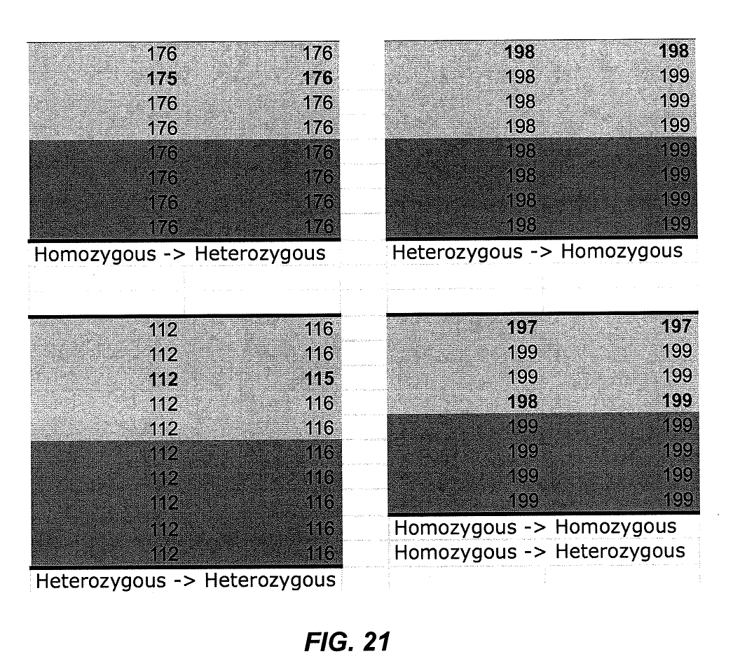

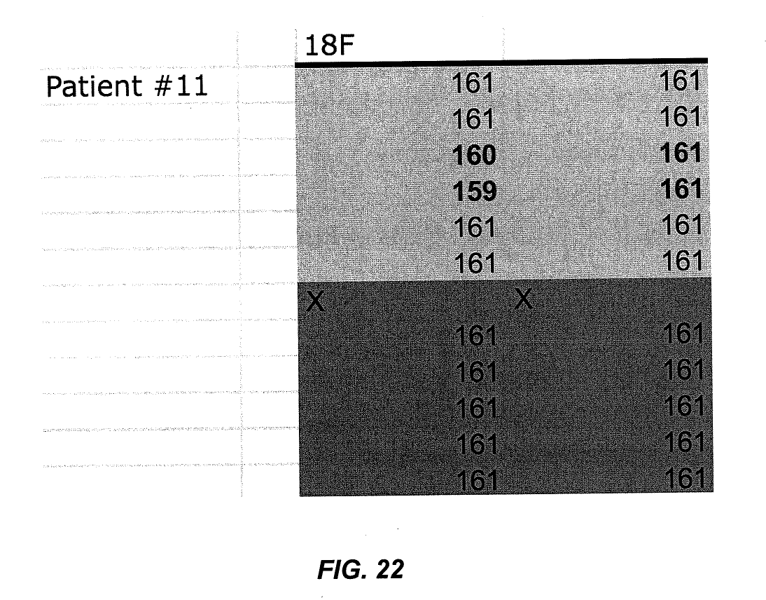

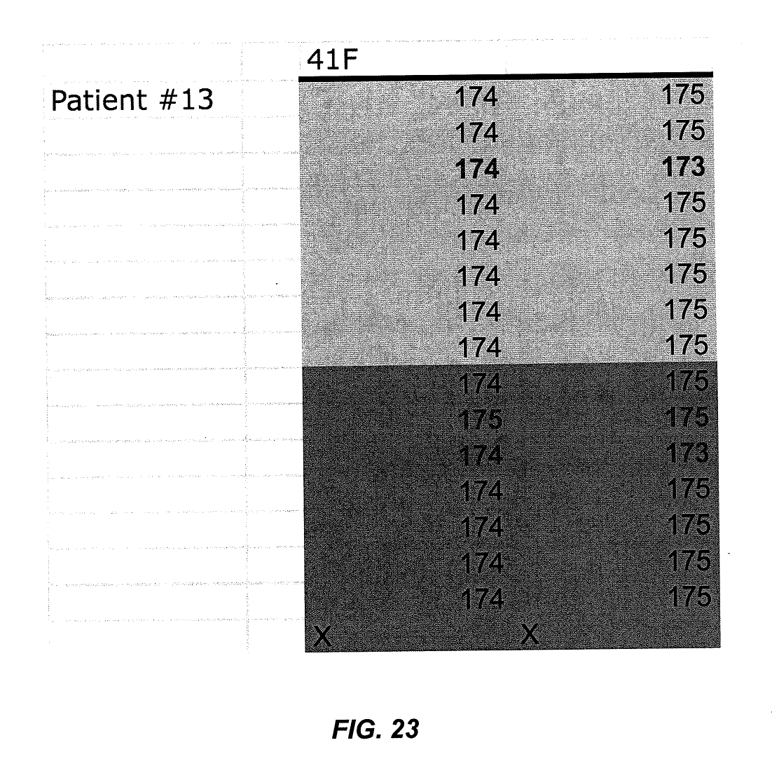

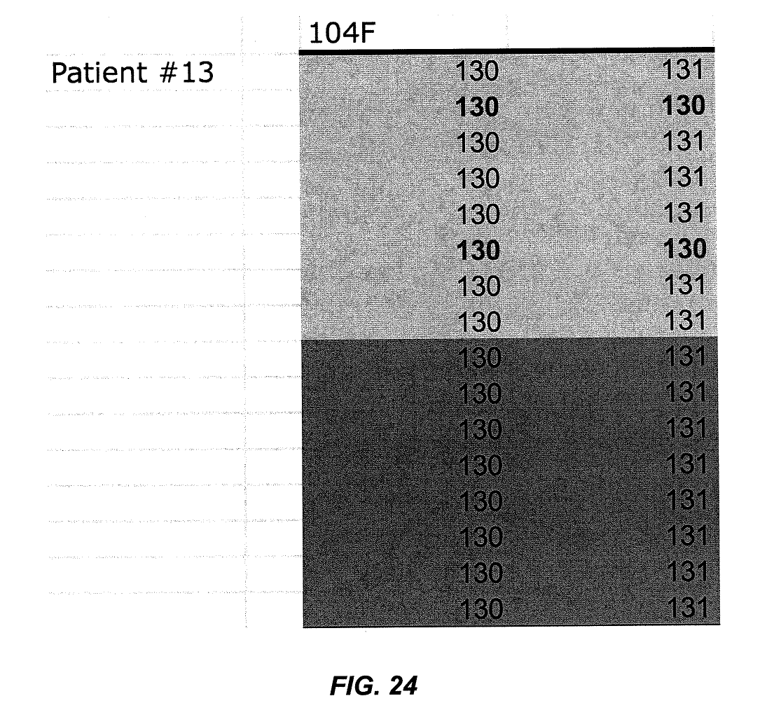

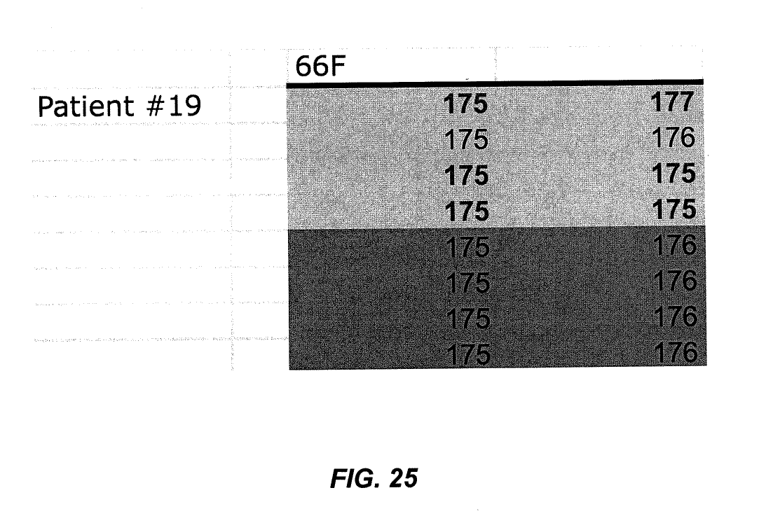

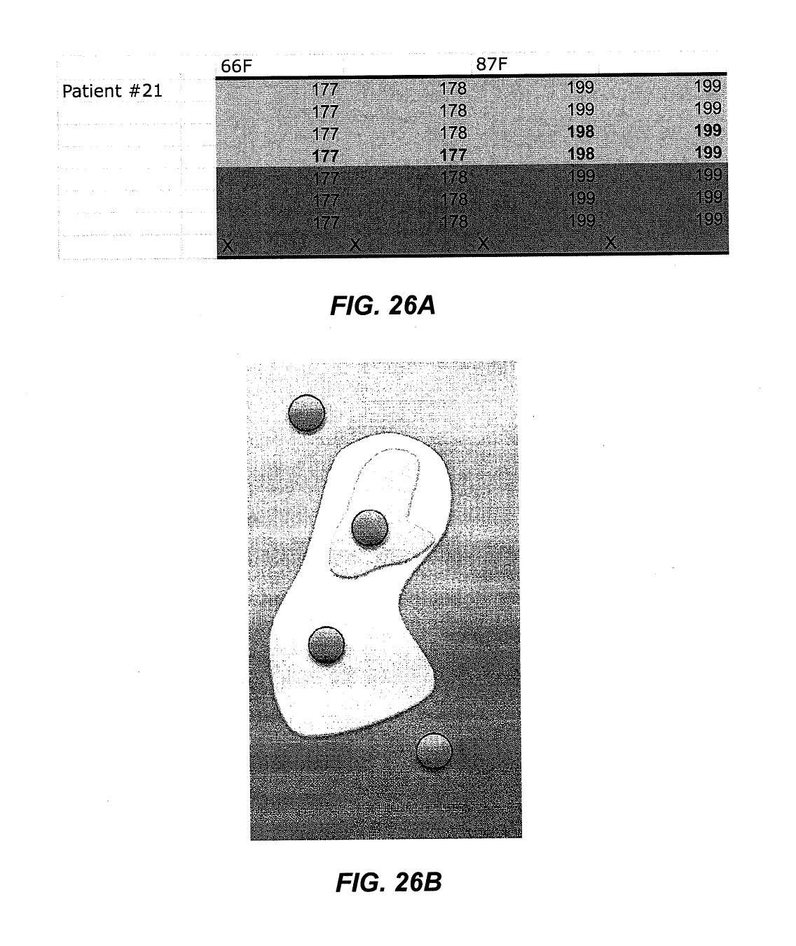

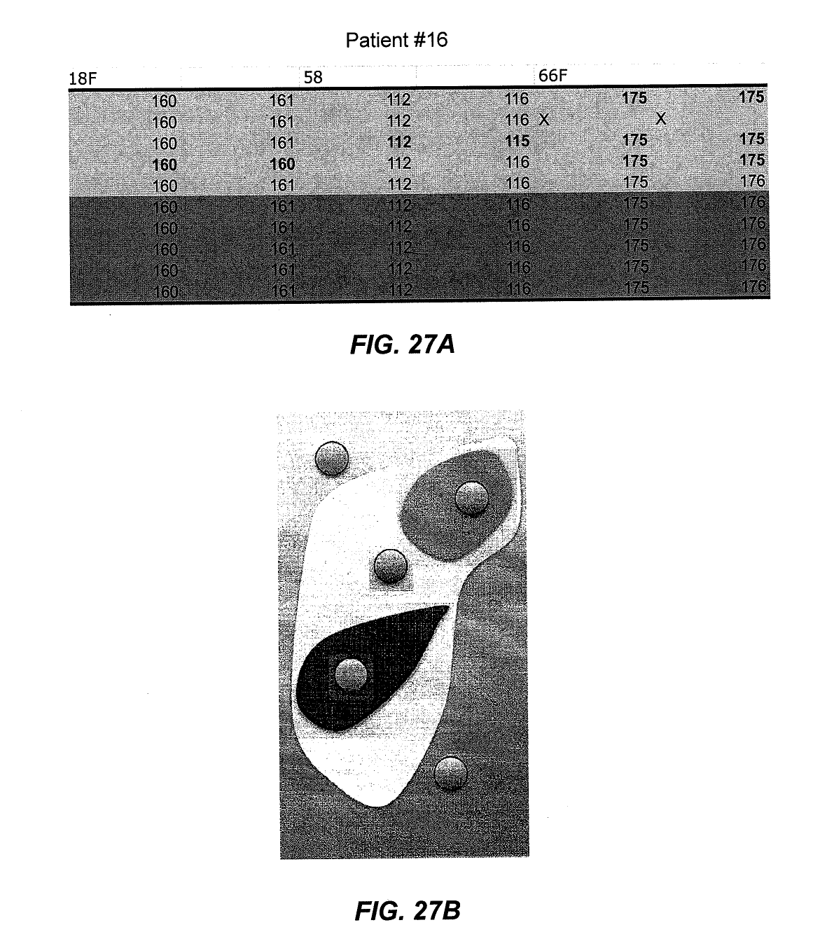

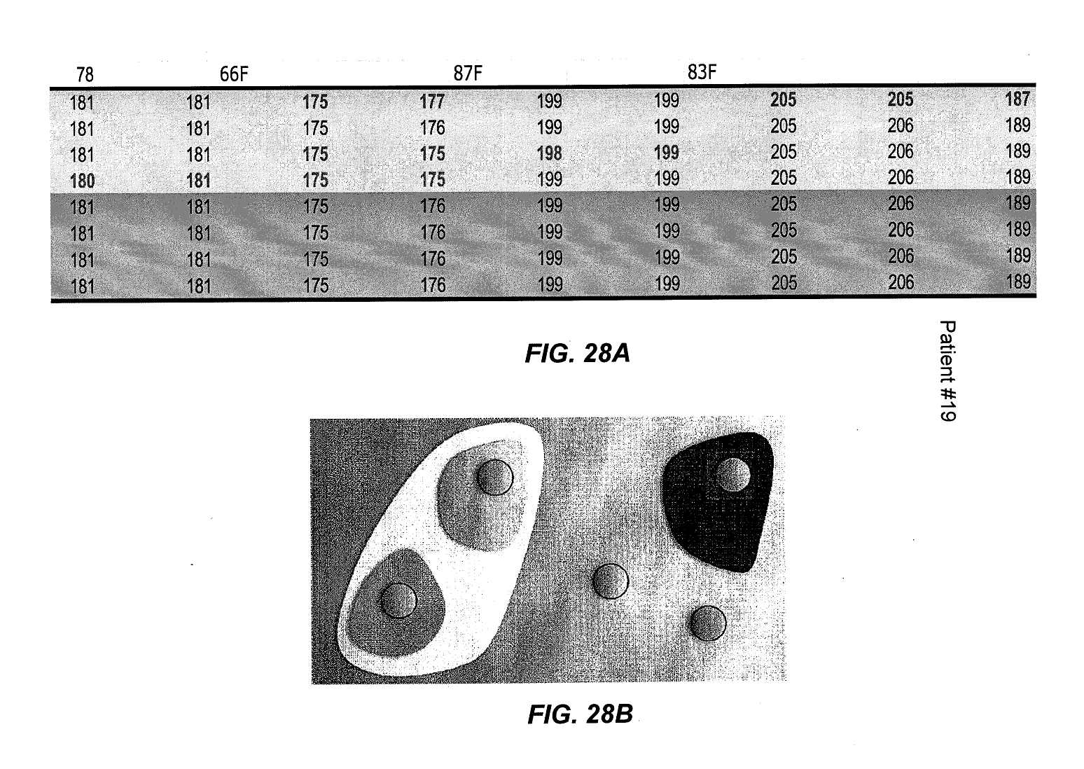

As can be seen in FIGS. 4-9, with the possible exception of PSC status, there are no other objective factors considered that are associated with the number of detectable slippage events. Examples of data for individual patients at amplicons (marker loci) showing slippage events can be found in FIGS. 17 to 28.

TABLE 1
List of amplicons, primers, and genetic loci used in the association study.

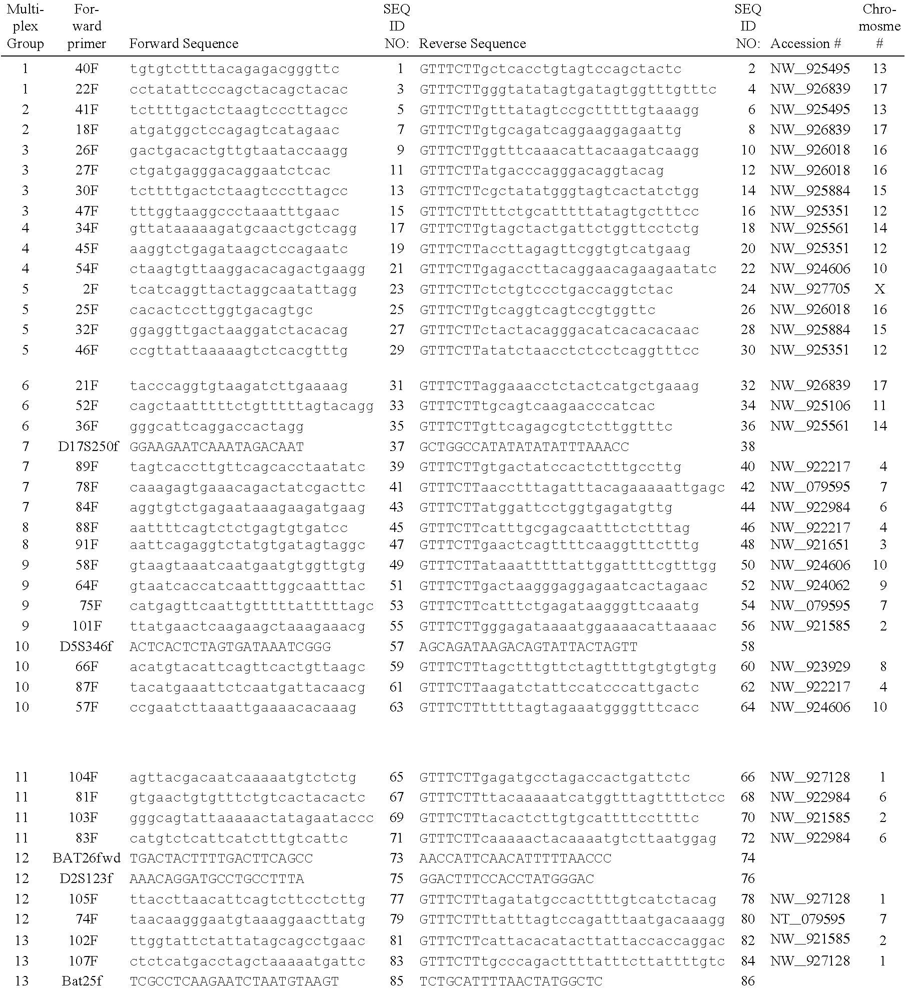

* Shaded rows indicate the Bethesda panel markers
** For the non-Bethesda markers, the GTTCTT stretech listed in bold is an extra tail added, to the reverse primer sequence for dye labeling purposes

TABLE 2
Complete representative data set for patient 1.

| | | | | | | | Blind | Blind | | | |
|---|---|---|---|---|---|---|---|---|---|---|---|
| | | | | | Study | Specimen | individual | sample | | Overall | DX |
| Case | Location | Tissue | Type | Source | ID | ID | # | # | Origin | DX | biopsy |
| Normal | Rectal | Epithelium | Fresh | Rosana rectal | 51782 | 480198 | 1 | 1 | ? | NEG | NEG |
| colon | | | | bank Aug. 26, 2008 | | | | | | | |
| Normal | Rectal | Epithelium | Fresh | Rosana rectal | 51782 | 480201 | 1 | 2 | ? | NEG | NEG |
| colon | | | | bank Aug. 26, 2008 | | | | | | | |
| Normal | Rectal | Epithelium | Fresh | Rosana rectal | 51782 | 480204 | 1 | 3 | ? | NEG | NEG |
| colon | | | | bank Aug. 26, 2008 | | | | | | | |
| Normal | Rectal | Epithelium | Fresh | Rosana rectal | 51782 | 480207 | 1 | 4 | ? | NEG | NEG |
| colon | | | | bank Aug. 26, 2008 | | | | | | | |
| Normal | Rectal | Stroma | Fresh | Rosana rectal | 51782 | 480198 | 1 | 5 | ? | NEG | NEG |
| colon | | | | bank Aug. 26, 2008 | | | | | | | |
| Normal | Rectal | Stroma | Fresh | Rosana rectal | 51782 | 480201 | 1 | 6 | ? | NEG | NEG |
| colon | | | | bank Aug. 26, 2008 | | | | | | | |
| Normal | Rectal | Stroma | Fresh | Rosana rectal | 51782 | 480204 | 1 | 7 | ? | NEG | NEG |
| colon | | | | bank Aug. 26, 2008 | | | | | | | |
| Normal | Rectal | Stroma | Fresh | Rosana rectal | 51782 | 480207 | 1 | 8 | ? | NEG | NEG |
| colon | | | | bank Aug. 26, 2008 | | | | | | | |

| | | | | | | | | | Cm | | Horizontal | Total |
|---|---|---|---|---|---|---|---|---|---|---|---|---|
| | PSC | | | | Disease | | | | from | Vertical cm | cm to | distance to |
| Case | status | Gender | Age | Duration | activity | Inflammation | Infiltrates | Aggregates | rectum | to dysplasia | dysplasia | dysplasia |
| Normal | N | F | 60 | — | — | — | — | — | R | — | — | — |
| colon | | | | | | | | | | | | |
| Normal | N | F | 60 | — | — | — | — | — | R | — | — | — |
| colon | | | | | | | | | | | | |
| Normal | N | F | 60 | — | — | — | — | — | R | — | — | — |
| colon | | | | | | | | | | | | |
| Normal | N | F | 60 | — | — | — | — | — | R | — | — | — |
| colon | | | | | | | | | | | | |
| Normal | N | F | 60 | — | — | — | — | — | R | — | — | — |
| colon | | | | | | | | | | | | |
| Normal | N | F | 60 | — | — | — | — | — | R | — | — | — |
| colon | | | | | | | | | | | | |
| Normal | N | F | 60 | — | — | — | — | — | R | — | — | — |
| colon | | | | | | | | | | | | |
| Normal | N | F | 60 | — | — | — | — | — | R | — | — | — |
| colon | | | | | | | | | | | | |

| | 41F-21F | 41F-21F | 41F-21F | D17S250f- | D17S250f-BAT25f | D17S250f-BAT25f | | | | |
|---|---|---|---|---|---|---|---|---|---|---|
| Case | plate | ng/well | 1st well | BAT25f plate | ng/well | 1st well | 41F | 41F | 18F | 18F |
| Normal | J. Salk | | | J. Salk 003 | 4 | AI | X | X | 160 | 161 |
| colon | 001 | | | | | | | | | |
| Normal | J. Salk | | | J. Salk 003 | 4 | C1 | X | X | 160 | 161 |
| colon | 001 | | | | | | | | | |
| Normal | J. Salk | | | J. Salk 003 | 2.9 | E1 | X | X | 160 | 161 |
| colon | 001 | | | | | | | | | |
| Normal | J. Salk | | | J. Salk 003 | 4 | G1 | X | X | 160 | 161 |
| colon | 001 | | | | | | | | | |
| Normal | J. Salk | | | J. Salk 003 | 4 | I1 | 170 | 171 | 160 | 161 |
| colon | 001 | | | | | | | | | |
| Normal | J. Salk | | | J. Salk 003 | 4 | K1 | 170 | 171 | 160 | 161 |
| colon | 001 | | | | | | | | | |
| Normal | J. Salk | | | J. Salk 003 | 4 | M1 | 170 | 171 | X | X |
| colon | 001 | | | | | | | | | |
| Normal | J. Salk | | | J. Salk 003 | 4 | O1 | 170 | 171 | X | X |
| colon | 001 | | | | | | | | | |

| Case | 26F | 26F | 27F | 27F | 30F | 30F | 47F | 47F | 45F | 45F | 54F | 54F | 34F | 34F | 46F | 46F |
|---|---|---|---|---|---|---|---|---|---|---|---|---|---|---|---|---|
| Normal | 155 | 155 | 99 | 100 | X | X | X | X | X | X | X | X | X | X | 162 | 162 |
| colon | | | | | | | | | | | | | | | | |
| Normal | 155 | 155 | 99 | 100 | X | X | 177 | 178 | X | X | 165 | 166 | X | X | 162 | 162 |
| colon | | | | | | | | | | | | | | | | |
| Normal | 155 | 155 | 99 | 100 | 117 | 118 | 177 | 178 | X | X | 165 | 166 | X | X | X | X |
| colon | | | | | | | | | | | | | | | | |
| Normal | 155 | 155 | X | X | 117 | 118 | 177 | 178 | X | X | 165 | 166 | X | X | X | X |
| colon | | | | | | | | | | | | | | | | |
| Normal | 155 | 155 | 99 | 100 | 117 | 118 | 177 | 178 | X | X | X | X | X | X | X | X |
| colon | | | | | | | | | | | | | | | | |
| Normal | 155 | 155 | 99 | 100 | X | X | 177 | 178 | X | X | 165 | 166 | 147 | 148 | X | X |
| colon | | | | | | | | | | | | | | | | |
| Normal | 155 | 155 | 99 | 100 | 117 | 118 | 177 | 178 | X | X | X | X | X | X | X | X |
| colon | | | | | | | | | | | | | | | | |
| Normal | 155 | 155 | 99 | 100 | 117 | 118 | 177 | 178 | X | X | 165 | 166 | 147 | 148 | X | X |
| colon | | | | | | | | | | | | | | | | |

| Case | 2F | 2F | 25F | 25F | 32F | 32F | 2F | 2F | 36F | 36F | 21F | 21F | D17S250f | D17S250f | 78 | 78 | 58 | 58 |
|---|---|---|---|---|---|---|---|---|---|---|---|---|---|---|---|---|---|---|
| Normal | X | X | X | X | X | X | X | X | X | X | X | X | 148 | 149 | 181 | 181 | 114 | 116 |
| colon | | | | | | | | | | | | | | | | | | |
| Normal | 115 | 115 | X | X | X | X | 180 | 180 | X | X | X | X | 148 | 149 | 181 | 181 | 114 | 116 |
| colon | | | | | | | | | | | | | | | | | | |
| Normal | X | X | X | X | X | X | 180 | 180 | X | X | X | X | 148 | 149 | 181 | 181 | 114 | 116 |
| colon | | | | | | | | | | | | | | | | | | |
| Normal | 115 | 115 | 93 | 94 | X | X | 180 | 180 | 123 | 123 | X | X | 148 | 149 | 181 | 181 | 114 | 116 |
| colon | | | | | | | | | | | | | | | | | | |
| Normal | X | X | X | X | X | X | 180 | 180 | X | X | 160 | 161 | 148 | 149 | 181 | 181 | 114 | 116 |
| colon | | | | | | | | | | | | | | | | | | |
| Normal | X | X | X | X | X | X | 180 | 180 | 123 | 123 | 160 | 161 | 148 | 149 | 181 | 181 | 114 | 116 |
| colon | | | | | | | | | | | | | | | | | | |
| Normal | X | X | X | X | X | X | 180 | 180 | 123 | 123 | 160 | 161 | 148 | 149 | 181 | 181 | 114 | 116 |
| colon | | | | | | | | | | | | | | | | | | |
| Normal | X | X | X | X | X | X | 180 | 180 | 123 | 123 | 160 | 161 | 148 | 149 | 181 | 181 | 114 | 116 |
| colon | | | | | | | | | | | | | | | | | | |

| Case | 64 | 64 | D5S346f | D5S346f | 66F | 66F | 87F | 87F | 104F | 104F | 81F | 81F | 103F | 103F | 83F | 83F |
|---|---|---|---|---|---|---|---|---|---|---|---|---|---|---|---|---|
| Normal | 152 | 152 | 109 | 109 | 176 | 176 | 200 | 200 | 129 | 130 | 163 | 163 | 175 | 175 | 206 | 206 |
| colon | | | | | | | | | | | | | | | | |
| Normal | 152 | 152 | 109 | 109 | 175 | 176 | 200 | 200 | 129 | 130 | X | X | 175 | 175 | 206 | 206 |
| colon | | | | | | | | | | | | | | | | |
| Normal | 152 | 152 | 109 | 109 | 176 | 176 | 200 | 200 | 129 | 130 | 163 | 163 | 175 | 175 | 206 | 206 |
| colon | | | | | | | | | | | | | | | | |
| Normal | 152 | 152 | 109 | 109 | 176 | 176 | 200 | 200 | 129 | 130 | X | X | 175 | 175 | 206 | 206 |
| colon | | | | | | | | | | | | | | | | |
| Normal | 152 | 152 | X | X | 176 | 176 | 200 | 200 | 129 | 130 | X | X | 175 | 175 | 206 | 206 |
| colon | | | | | | | | | | | | | | | | |
| Normal | 152 | 152 | 109 | 109 | 176 | 176 | 200 | 200 | 129 | 130 | X | X | 175 | 175 | 206 | 206 |
| colon | | | | | | | | | | | | | | | | |
| Normal | X | X | 109 | 109 | 176 | 176 | 200 | 200 | 129 | 130 | 163 | 163 | 175 | 175 | 206 | 206 |
| colon | | | | | | | | | | | | | | | | |
| Normal | X | X | 109 | 109 | 176 | 176 | X | X | X | X | 163 | 163 | 175 | 175 | 206 | 206 |
| colon | | | | | | | | | | | | | | | | |

| Case | BAT26f | BAT26f | 103F | 103F | 102F | 102F | 107F | 107F | BAT25f | BAT25f | Reads | Total | Attempts | % Success |
|---|---|---|---|---|---|---|---|---|---|---|---|---|---|---|
| Normal | 116 | 116 | 188 | 189 | X | X | 115 | 115 | 123 | 123 | 19 | | | |
| colon | | | | | | | | | | | | | | |
| Normal | 116 | 116 | 188 | 189 | 155 | 155 | 115 | 115 | 123 | 123 | 23 | | | |
| colon | | | | | | | | | | | | | | |
| Normal | 116 | 116 | 188 | 189 | 155 | 155 | 115 | 115 | 123 | 123 | 23 | | | |
| colon | | | | | | | | | | | | | | |
| Normal | 116 | 116 | 188 | 189 | 155 | 155 | 115 | 115 | 123 | 123 | 24 | | | |
| colon | | | | | | | | | | | | | | |
| Normal | 116 | 116 | 188 | 189 | 155 | 155 | 115 | 115 | 123 | 123 | 22 | | | |
| colon | | | | | | | | | | | | | | |
| Normal | 116 | 116 | 188 | 189 | 155 | 155 | 115 | 115 | 123 | 123 | 25 | | | |
| colon | | | | | | | | | | | | | | |
| Normal | 116 | 116 | 188 | 189 | 155 | 155 | 115 | 115 | 123 | 123 | 23 | | | |
| colon | | | | | | | | | | | | | | |
| Normal | 116 | 116 | 188 | 189 | X | X | 115 | 115 | 123 | 123 | 22 | 181 | 256 | 70.7% |
| colon | | | | | | | | | | | | | | |

| | | Total mutations per | Total positive | Total | Total | Total | Mutants per |
|---|---|---|---|---|---|---|---|
| Case | Mutants | biopsy | biopsies | epithelium | stroma | mutations | read |
| Normal | | | | | | | |
| colon | | | | | | | |
| Normal | I | 1 | | | | | |
| colon | | | | | | | |
| Normal | | | | | | | |
| colon | | | | | | | |
| Normal | | | | | | | |
| colon | | | | | | | |
| Normal | | | | | | | |
| colon | | | | | | | |
| Normal | | | | | | | |
| colon | | | | | | | |
| Normal | | | | | | | |
| colon | | | | | | | |
| Normal | | | ¼ | 1 | 0 | 1 | 0.55% |
| colon | | | | | | | |

TABLE 3
Complete representative data set for patient 4.

| | | | | | Study | Specimen | Blind | Blind | | Overall |
|---|---|---|---|---|---|---|---|---|---|---|
| Case | Location | Tissue | Type | Source | ID | ID | individual # | sample # | Origin | DX |
| UC non- | Rectal | Epithelium | Fresh | Rosana | 52238 | 303165 | 4 | 1 | ? | NEG |
| progressor | | | | rectal bank | | | | | | |
| | | | | Aug. 26, 2008 | | | | | | |
| UC non- | Rectal | Epithelium | Fresh | Rosana | 52238 | 303168 | 4 | 2 | ? | NEG |
| progressor | | | | rectal bank | | | | | | |
| | | | | Aug. 26, 2008 | | | | | | |
| UC non- | Rectal | Epithelium | Fresh | Rosana | 52238 | 303171 | 4 | 3 | ? | NEG |
| progressor | | | | rectal bank | | | | | | |
| | | | | Aug. 26, 2008 | | | | | | |
| UC non- | Rectal | Epithelium | Fresh | Rosana | 52238 | 303174 | 4 | 4 | ? | NEG |
| progressor | | | | rectal bank | | | | | | |
| | | | | Aug. 26, 2008 | | | | | | |
| UC non- | Rectal | Stroma | Fresh | Rosana | 52238 | 303165 | 4 | 5 | ? | NEG |
| progressor | | | | rectal bank | | | | | | |
| | | | | Aug. 26, 2008 | | | | | | |
| UC non- | Rectal | Stroma | Fresh | Rosana | 52238 | 303168 | 4 | 6 | ? | NEG |
| progressor | | | | rectal bank | | | | | | |
| | | | | Aug. 26, 2008 | | | | | | |
| UC non- | Rectal | Stroma | Fresh | Rosana | 52238 | 303171 | 4 | 7 | ? | NEG |
| progressor | | | | rectal bank | | | | | | |
| | | | | Aug. 26, 2008 | | | | | | |
| UC non- | Rectal | Stroma | Fresh | Rosana | 52238 | 303174 | 4 | 8 | ? | NEG |
| progressor | | | | rectal bank | | | | | | |
| | | | | Aug. 26, 2008 | | | | | | |

| | | | | | | | | | | | | | Total |
|---|---|---|---|---|---|---|---|---|---|---|---|---|---|
| | | | | | | | | | | Cm | Vertical | Horizontal | distance |
| | DX | PSC | | | Dura- | Disease | | | | from | cm to | cm to | to |
| Case | biopsy | status | Gender | Age | tion | activity | Inflammation | Infiltrates | Aggregates | rectum | dysplasia | dysplasia | dysplasia |
| UC non- | NEG | Y | M | 43 | 2 | Severe | 3 | ? | ? | R | — | — | — |
| progressor | | | | | | | | | | | | | |
| UC non- | NEG | Y | M | 43 | 2 | Severe | 3 | ? | ? | R | — | — | — |
| progressor | | | | | | | | | | | | | |
| UC non- | NEG | Y | M | 43 | 2 | Severe | 3 | ? | ? | R | — | — | — |
| progressor | | | | | | | | | | | | | |
| UC non- | NEG | Y | M | 43 | 2 | Severe | 3 | ? | ? | R | — | — | — |
| progressor | | | | | | | | | | | | | |
| UC non- | NEG | Y | M | 43 | 2 | Severe | 3 | ? | ? | R | — | — | — |
| progressor | | | | | | | | | | | | | |
| UC non- | NEG | Y | M | 43 | 2 | Severe | 3 | ? | ? | R | — | — | — |
| progressor | | | | | | | | | | | | | |
| UC non- | NEG | Y | M | 43 | 2 | Severe | 3 | ? | ? | R | — | — | — |
| progressor | | | | | | | | | | | | | |
| UC non- | NEG | Y | M | 43 | 2 | Severe | 3 | ? | ? | R | — | — | — |
| progressor | | | | | | | | | | | | | |

| | | 41F- | 41F- | | | | | | | | | | | | | |
|---|---|---|---|---|---|---|---|---|---|---|---|---|---|---|---|---|
| | 41F- | 21F | 21F | D17S250f- | D17S250f- | D17S250f- | | | | | | | | | | |
| | 21F | ng/ | 1st | BAT25f | BAT25f | BAT25f | | | | | | | | | | |
| Case | plate | well | well | plate | ng/well | 1st well | 41F | 41F | 18F | 18F | 26F | 26F | 27F | 27F | 30F | 30F |
| UC non- | J. Salk | | | J. Salk 003 | 4 | A4 | X | X | X | X | 157 | 175 | 100 | 100 | X | X |
| progressor | 001 | | | | | | | | | | | | | | | |
| UC non- | J. Salk | | | J. Salk 003 | 4 | C4 | X | X | X | X | 157 | 175 | 100 | 100 | X | X |
| progressor | 001 | | | | | | | | | | | | | | | |
| UC non- | J. Salk | | | J. Salk 003 | 4 | E4 | X | X | X | X | 157 | 175 | 100 | 100 | X | X |
| progressor | 001 | | | | | | | | | | | | | | | |
| UC non- | J. Salk | | | J. Salk 003 | 4 | G4 | X | X | 160 | 161 | 157 | 175 | 100 | 100 | X | X |
| progressor | 001 | | | | | | | | | | | | | | | |
| UC non- | J. Salk | | | J. Salk 003 | 4 | I4 | X | X | X | X | 157 | 175 | 100 | 100 | X | X |
| progressor | 001 | | | | | | | | | | | | | | | |
| UC non- | J. Salk | | | J. Salk 003 | 4 | K4 | X | X | X | X | 157 | 175 | 100 | 100 | X | X |
| progressor | 001 | | | | | | | | | | | | | | | |
| UC non- | J. Salk | | | J. Salk 003 | 4 | M4 | X | X | X | X | 157 | 175 | 100 | 100 | X | X |
| progressor | 001 | | | | | | | | | | | | | | | |
| UC non- | J. Salk | | | J. Salk 003 | 4 | O4 | X | X | 160 | 161 | 157 | 175 | 100 | 100 | X | X |
| progressor | 001 | | | | | | | | | | | | | | | |

| Case | 47F | 47F | 45F | 45F | 54F | 54F | 34F | 34F | 46F | 46F | 2F | 2F | 25F | 25F | 32F | 32F | 52F | 52F | 36F |
|---|---|---|---|---|---|---|---|---|---|---|---|---|---|---|---|---|---|---|---|
| UC non- | 177 | 178 | X | X | 166 | 167 | X | X | 160 | 161 | 115 | 115 | 93 | 94 | X | X | 179 | 179 | 114 |
| progressor | | | | | | | | | | | | | | | | | | | |
| UC non- | 177 | 178 | X | X | X | X | X | X | 160 | 161 | 115 | 115 | 93 | 94 | X | X | 179 | 179 | 114 |
| progressor | | | | | | | | | | | | | | | | | | | |
| UC non- | 177 | 178 | X | X | 166 | 167 | X | X | X | X | X | X | X | X | X | X | 179 | 179 | 114 |
| progressor | | | | | | | | | | | | | | | | | | | |
| UC non- | 177 | 178 | X | X | X | X | X | X | X | X | X | X | X | X | 183 | 187 | 179 | 179 | 114 |
| progressor | | | | | | | | | | | | | | | | | | | |
| UC non- | 177 | 178 | X | X | X | X | X | X | 160 | 161 | 115 | 115 | 93 | 94 | X | X | 179 | 179 | 114 |
| progressor | | | | | | | | | | | | | | | | | | | |
| UC non- | 177 | 178 | X | X | 166 | 167 | 147 | 148 | 160 | 161 | 115 | 115 | 93 | 94 | X | X | 179 | 179 | 114 |
| progressor | | | | | | | | | | | | | | | | | | | |
| UC non- | 177 | 178 | X | X | 166 | 167 | 147 | 148 | 160 | 161 | 115 | 115 | X | X | X | X | 179 | 179 | 114 |
| progressor | | | | | | | | | | | | | | | | | | | |
| UC non- | 177 | 178 | X | X | 166 | 167 | 147 | 148 | X | X | X | X | X | X | X | X | 179 | 179 | 114 |
| progressor | | | | | | | | | | | | | | | | | | | |

| Case | 36F | 21F | 21F | D17S250f | D17S250f | 78 | 78 | 58 | 58 | 64 | 64 | D5S346f | D5S346f | 66F | 66F | 87F |
|---|---|---|---|---|---|---|---|---|---|---|---|---|---|---|---|---|
| UC non- | 114 | 160 | 161 | 148 | 160 | 181 | 182 | 114 | 116 | 151 | 152 | 119 | 121 | 176 | 177 | 196 |
| progressor | | | | | | | | | | | | | | | | |
| UC non- | 114 | 160 | 161 | 148 | 160 | 181 | 182 | 114 | 116 | 151 | 152 | 119 | 121 | 176 | 177 | 196 |
| progressor | | | | | | | | | | | | | | | | |
| UC non- | 114 | 160 | 161 | 148 | 160 | 181 | 182 | X | X | 151 | 152 | 119 | 121 | 176 | 177 | 196 |
| progressor | | | | | | | | | | | | | | | | |
| UC non- | 114 | 160 | 161 | 148 | 160 | 181 | 182 | 114 | 116 | 151 | 152 | 119 | 121 | 176 | 177 | 196 |
| progressor | | | | | | | | | | | | | | | | |
| UC non- | 114 | 160 | 161 | 148 | 160 | 181 | 182 | 114 | 116 | 151 | 152 | 119 | 121 | 176 | 177 | 196 |
| progressor | | | | | | | | | | | | | | | | |
| UC non- | 114 | 160 | 161 | 148 | 160 | 181 | 182 | 114 | 116 | 151 | 152 | 119 | 121 | 176 | 177 | 196 |
| progressor | | | | | | | | | | | | | | | | |
| UC non- | 114 | 160 | 161 | 148 | 160 | 181 | 182 | 114 | 116 | 151 | 152 | 119 | 121 | 176 | 177 | 196 |
| progressor | | | | | | | | | | | | | | | | |
| UC non- | 114 | 160 | 161 | 148 | 160 | 181 | 182 | 114 | 116 | 151 | 152 | 119 | 121 | 176 | 177 | 196 |
| progressor | | | | | | | | | | | | | | | | |

| Case | 87F | 104F | 104F | 81F | 81F | 103F | 103F | 83F | 83F | BAT26f | BAT26f | 103F | 103F | 102F | 102F | 107F | 107F |
|---|---|---|---|---|---|---|---|---|---|---|---|---|---|---|---|---|---|
| UC non- | 196 | 127 | 128 | 155 | 155 | 175 | 175 | 206 | 207 | 117 | 118 | X | X | 155 | 155 | 117 | 117 |
| progressor | | | | | | | | | | | | | | | | | |
| UC non- | 196 | 127 | 128 | 155 | 155 | 175 | 175 | 206 | 207 | 117 | 118 | X | X | 155 | 155 | 117 | 117 |
| progressor | | | | | | | | | | | | | | | | | |
| UC non- | 196 | 127 | 128 | 155 | 155 | 175 | 175 | 206 | 207 | 117 | 118 | X | X | 155 | 155 | 117 | 117 |
| progressor | | | | | | | | | | | | | | | | | |
| UC non- | 196 | 127 | 128 | 155 | 155 | 175 | 175 | 206 | 207 | 117 | 118 | X | X | 155 | 155 | 117 | 117 |
| progressor | | | | | | | | | | | | | | | | | |
| UC non- | 196 | 127 | 128 | 155 | 155 | 175 | 175 | 206 | 207 | 117 | 118 | X | X | 155 | 155 | 117 | 117 |
| progressor | | | | | | | | | | | | | | | | | |
| UC non- | 196 | 127 | 128 | 155 | 155 | 175 | 175 | 206 | 207 | 117 | 118 | X | X | 155 | 155 | 117 | 117 |
| progressor | | | | | | | | | | | | | | | | | |
| UC non- | 196 | 127 | 128 | 155 | 155 | 175 | 175 | 206 | 207 | 117 | 118 | X | X | 155 | 155 | 117 | 117 |
| progressor | | | | | | | | | | | | | | | | | |
| UC non- | 196 | 127 | 128 | 155 | 155 | 175 | 175 | 206 | 207 | 117 | 118 | X | X | 155 | 155 | 117 | 117 |
| progressor | | | | | | | | | | | | | | | | | |

| | | | | | | | | Total | | | | | |
|---|---|---|---|---|---|---|---|---|---|---|---|---|---|
| | | | | | | | | mutations | Total | | | | |
| | | | | | | % | | per | positive | Total | Total | Total | Mutants |
| Case | BAT25f | BAT25f | Reads | Total | Attempts | Success | Mutants | biopsy | biopsies | epithelium | stroma | mutations | per read |
| UC non- | 122 | 123 | 25 | | | | | | | | | | |
| progressor | | | | | | | | | | | | | |
| UC non- | 122 | 123 | 24 | | | | | | | | | | |
| progressor | | | | | | | | | | | | | |
| UC non- | 122 | 123 | 21 | | | | | | | | | | |
| progressor | | | | | | | | | | | | | |
| UC non- | 122 | 123 | 23 | | | | | | | | | | |
| progressor | | | | | | | | | | | | | |
| UC non- | 122 | 123 | 24 | | | | | | | | | | |
| progressor | | | | | | | | | | | | | |
| UC non- | 122 | 123 | 26 | | | | | | | | | | |
| progressor | | | | | | | | | | | | | |
| UC non- | 122 | 123 | 25 | | | | | | | | | | |
| progressor | | | | | | | | | | | | | |
| UC non- | 122 | 123 | 24 | 192 | 256 | 75.0% | | | 0/4 | 0 | 0 | 0 | 0.00% |
| progressor | | | | | | | | | | | | | |

TABLE 4
Complete representative data set for patient 11.

| | | | | | Study | Specimen | Blind | Blind | | Overall | DX |
|---|---|---|---|---|---|---|---|---|---|---|---|
| Case | Location | Tissue | Type | Source | ID | ID | individual # | sample # | Origin | DX | biopsy |
| UC with | Colon | Epithelium | Fresh | Rosana | 183L | C29 | 11 | 1 | UW | HGD | NEG |
| adjacent | | | | Mapping | | | | | | | |
| HGD | | | | study | | | | | | | |
| | | | | Aug. 26, 2008 | | | | | | | |
| UC with | Colon | Epithelium | Fresh | Rosana | 183L | B24 | 11 | 2 | UW | HGD | NEG |
| adjacent | | | | Mapping | | | | | | | |
| HGD | | | | study | | | | | | | |
| | | | | Aug. 26, 2008 | | | | | | | |
| UC with | Colon | Epithelium | Fresh | Rosana | 183L | D18 | 11 | 3 | UW | HGD | NEG |
| adjacent | | | | Mapping | | | | | | | |
| HGD | | | | study | | | | | | | |
| | | | | Aug. 26, 2008 | | | | | | | |
| UC with | Colon | Epithelium | Fresh | Rosana | 183L | A7 | 11 | 4 | UW | HGD | NEG |
| adjacent | | | | Mapping | | | | | | | |
| HGD | | | | study | | | | | | | |
| | | | | Aug. 26, 2008 | | | | | | | |
| UC with | Colon | Epithelium | Fresh | Rosana | 183L | B12 | 11 | 5 | UW | HGD | NEG |
| adjacent | | | | Mapping | | | | | | | |
| HGD | | | | study | | | | | | | |
| | | | | Aug. 26, 2008 | | | | | | | |
| UC with | Colon | Epithelium | Fresh | Rosana | 183L | A2 | 11 | 6 | UW | HGD | NEG |
| adjacent | | | | Mapping | | | | | | | |
| HGD | | | | study | | | | | | | |
| | | | | Aug. 26, 2008 | | | | | | | |
| UC with | Colon | Stroma | Fresh | Rosana | 183L | C29 | 11 | 7 | UW | HGD | NEG |
| adjacent | | | | Mapping | | | | | | | |
| HGD | | | | study | | | | | | | |
| | | | | Aug. 26, 2008 | | | | | | | |
| UC with | Colon | Stroma | Fresh | Rosana | 183L | B24 | 11 | 8 | UW | HGD | NEG |
| adjacent | | | | Mapping | | | | | | | |
| HGD | | | | study | | | | | | | |
| | | | | Aug. 26, 2008 | | | | | | | |
| UC with | Colon | Stroma | Fresh | Rosana | 183L | D18 | 11 | 9 | UW | HGD | NEG |
| adjacent | | | | Mapping | | | | | | | |
| HGD | | | | study | | | | | | | |
| | | | | Aug. 26, 2008 | | | | | | | |
| UC with | Colon | Stroma | Fresh | Rosana | 183L | A7 | 11 | 10 | UW | HGD | NEG |
| adjacent | | | | Mapping | | | | | | | |
| HGD | | | | study | | | | | | | |
| | | | | Aug. 26, 2008 | | | | | | | |
| UC with | Colon | Stroma | Fresh | Rosana | 183L | B12 | 11 | 11 | UW | HGD | NEG |
| adjacent | | | | Mapping | | | | | | | |
| HGD | | | | study | | | | | | | |
| | | | | Aug. 26, 2008 | | | | | | | |
| UC with | Colon | Stroma | Fresh | Rosana | 183L | A2 | 11 | 12 | UW | HGD | NEG |
| adjacent | | | | Mapping | | | | | | | |
| HGD | | | | study | | | | | | | |
| | | | | Aug. 26, 2008 | | | | | | | |

| | | | | | | | | | | | | Total |
|---|---|---|---|---|---|---|---|---|---|---|---|---|
| | | | | | | | | | Cm | Vertical | Horizontal | distance |
| | PSC | | | | Disease | | | | from | cm to | cm to | to |
| Case | status | Gender | Age | Duration | activity | Inflammation | Infiltrates | Aggregtes | rectum | dysplasia | dysplasia | dysplasia |
| UC with | Y | M | 36 | 11 | ? | 0 | 2 | 3 | 3.4 | 78.3 | 2 | 78.3 |
| adjacent | | | | | | | | | | | | |
| HGD | | | | | | | | | | | | |
| UC with | Y | M | 36 | 11 | ? | 1 | 1 | 0 | 20.4 | 61.3 | 0 | 61.3 |
| adjacent | | | | | | | | | | | | |
| HGD | | | | | | | | | | | | |
| UC with | Y | M | 36 | 11 | ? | 0 | 2 | 0 | 40.9 | 40.8 | 4 | 41.0 |
| adjacent | | | | | | | | | | | | |
| HGD | | | | | | | | | | | | |
| UC with | Y | M | 36 | 11 | ? | 4 | | 2 | 78.3 | 3.4 | 2 | 3.9 |
| adjacent | | | | | | | | | | | | |
| HGD | | | | | | | | | | | | |
| UC with | Y | M | 36 | 11 | ? | 3 | 3 | | 61.3 | 20.4 | 0 | 20.4 |
| adjacent | | | | | | | | | | | | |
| HGD | | | | | | | | | | | | |
| UC with | Y | M | 36 | 11 | ? | 2 | 3 | 2 | 95.3 | 0 | 4 | 4.0 |
| adjacent | | | | | | | | | | | | |
| HGD | | | | | | | | | | | | |
| UC with | Y | M | 36 | 11 | ? | 0 | 2 | 3 | 3.4 | 78.3 | 2 | 78.3 |
| adjacent | | | | | | | | | | | | |
| HGD | | | | | | | | | | | | |
| UC with | Y | M | 36 | 11 | ? | 1 | 1 | 0 | 20.4 | 61.3 | 0 | 61.3 |
| adjacent | | | | | | | | | | | | |
| HGD | | | | | | | | | | | | |
| UC with | Y | M | 36 | 11 | ? | 0 | 2 | 0 | 40.9 | 40.8 | 4 | 41.0 |
| adjacent | | | | | | | | | | | | |
| HGD | | | | | | | | | | | | |
| UC with | Y | M | 36 | 11 | ? | 4 | | 2 | 78.3 | 3.4 | 2 | 3.9 |
| adjacent | | | | | | | | | | | | |
| HGD | | | | | | | | | | | | |
| UC with | Y | M | 36 | 11 | ? | 3 | 3 | | 61.3 | 20.4 | 0 | 20.4 |
| adjacent | | | | | | | | | | | | |
| HGD | | | | | | | | | | | | |
| UC with | Y | M | 36 | 11 | ? | 2 | 3 | 2 | 95.3 | 0 | 4 | 4.0 |
| adjacent | | | | | | | | | | | | |
| HGD | | | | | | | | | | | | |

| | 41F-21F | 41F-21F | 41F-21F | D17S250f- | D17S250f- | D17S250f- | | | | | |
|---|---|---|---|---|---|---|---|---|---|---|---|
| Case | plate | ng/well | 1st well | BAT25f plate | BAT25f ng/well | BAT25f 1st well | 41F | 41F | 18F | 18F | 26F |
| UC with | J. Salk | | | J. Salk 003 | 4 | M11 | X | X | 161 | 161 | 156 |
| adjacent | 001 | | | | | | | | | | |
| HGD | | | | | | | | | | | |
| UC with | J. Salk | | | J. Salk 003 | 4 | O11 | X | X | 161 | 161 | 156 |
| adjacent | 001 | | | | | | | | | | |
| HGD | | | | | | | | | | | |
| UC with | J. Salk | | | J. Salk 003 | 4 | A12 | X | X | 160 | 161 | 156 |
| adjacent | 001 | | | | | | | | | | |
| HGD | | | | | | | | | | | |
| UC with | J. Salk | | | J. Salk 003 | 4 | C12 | X | X | 159 | 161 | 156 |
| adjacent | 001 | | | | | | | | | | |
| HGD | | | | | | | | | | | |
| UC with | J. Salk | 4 | M1 | J. Salk 003 | 4 | E12 | X | X | 161 | 161 | |
| adjacent | 002 | | | | | | | | | | |
| HGD | | | | | | | | | | | |
| UC with | J. Salk | 4 | O1 | J. Salk 003 | 4 | G12 | X | X | 161 | 161 | |
| adjacent | 002 | | | | | | | | | | |
| HGD | | | | | | | | | | | |
| UC with | J. Salk | | | J. Salk 003 | 4 | I12 | X | X | X | X | 156 |
| adjacent | 001 | | | | | | | | | | |
| HGD | | | | | | | | | | | |
| UC with | J. Salk | | | J. Salk 003 | 4 | K12 | X | X | 161 | 161 | 156 |
| adjacent | 001 | | | | | | | | | | |
| HGD | | | | | | | | | | | |
| UC with | J. Salk | | | J. Salk 003 | 4 | M12 | X | X | 161 | 161 | 156 |
| adjacent | 001 | | | | | | | | | | |
| HGD | | | | | | | | | | | |
| UC with | J. Salk | | | J. Salk 003 | 4 | O12 | X | X | 161 | 161 | 156 |
| adjacent | 001 | | | | | | | | | | |
| HGD | | | | | | | | | | | |
| UC with | J. Salk | 4 | A2 | J. Salk 003 | 4 | A13 | X | X | 161 | 161 | |
| adjacent | 002 | | | | | | | | | | |
| HGD | | | | | | | | | | | |
| UC with | J. Salk | 4 | C2 | J. Salk 003 | 4 | C13 | X | X | 161 | 161 | |
| adjacent | 002 | | | | | | | | | | |
| HGD | | | | | | | | | | | |

| Case | 26F | 27F | 27F | 30F | 30F | 47F | 47F | 45F | 45F | 54F | 54F | 34F | 34F | 46F | 46F | 2F | 2F | 25F | 25F |
|---|---|---|---|---|---|---|---|---|---|---|---|---|---|---|---|---|---|---|---|
| UC with | 155 | 99 | 101 | 109 | 110 | 178 | 182 | X | X | X | X | X | X | X | X | 115 | 115 | 93 | 93 |
| adjacent | | | | | | | | | | | | | | | | | | | |
| HGD | | | | | | | | | | | | | | | | | | | |
| UC with | 155 | 99 | 100 | 109 | 110 | 178 | 182 | X | X | X | X | X | X | 160 | 162 | 115 | 115 | 93 | 93 |
| adjacent | | | | | | | | | | | | | | | | | | | |
| HGD | | | | | | | | | | | | | | | | | | | |
| UC with | 155 | 99 | 100 | 109 | 110 | X | X | 127 | 127 | 165 | 166 | X | X | 160 | 162 | 115 | 115 | 93 | 93 |
| adjacent | | | | | | | | | | | | | | | | | | | |
| HGD | | | | | | | | | | | | | | | | | | | |
| UC with | 155 | 99 | 100 | 109 | 110 | 178 | 182 | X | X | 165 | 166 | 147 | 148 | 160 | 162 | 115 | 115 | X | X |
| adjacent | | | | | | | | | | | | | | | | | | | |
| HGD | | | | | | | | | | | | | | | | | | | |
| UC with | | 99 | 100 | 109 | 110 | 178 | 182 | X | X | 165 | 166 | X | X | X | X | 115 | 115 | 93 | 93 |
| adjacent | | | | | | | | | | | | | | | | | | | |
| HGD | | | | | | | | | | | | | | | | | | | |
| UC with | | X | X | 109 | 110 | 178 | 182 | X | X | 165 | 166 | X | X | 160 | 162 | 115 | 115 | 93 | 93 |
| adjacent | | | | | | | | | | | | | | | | | | | |
| HGD | | | | | | | | | | | | | | | | | | | |
| UC with | 155 | 99 | 100 | 109 | 110 | 178 | 182 | X | X | 165 | 166 | 147 | 148 | 160 | 162 | 115 | 115 | 93 | 93 |
| adjacent | | | | | | | | | | | | | | | | | | | |
| HGD | | | | | | | | | | | | | | | | | | | |
| UC with | 155 | 99 | 100 | 109 | 110 | 178 | 182 | X | X | 165 | 166 | X | X | 160 | 162 | 115 | 115 | 93 | 93 |
| adjacent | | | | | | | | | | | | | | | | | | | |
| HGD | | | | | | | | | | | | | | | | | | | |
| UC with | 155 | 99 | 100 | 109 | 110 | 178 | 182 | X | X | 165 | 166 | X | X | X | X | 115 | 115 | 93 | 93 |
| adjacent | | | | | | | | | | | | | | | | | | | |
| HGD | | | | | | | | | | | | | | | | | | | |
| UC with | 155 | 99 | 100 | 109 | 110 | 178 | 182 | 127 | 127 | 165 | 166 | 147 | 148 | 160 | 162 | 115 | 115 | 93 | 93 |
| adjacent | | | | | | | | | | | | | | | | | | | |
| HGD | | | | | | | | | | | | | | | | | | | |
| UC with | | 99 | 100 | 109 | 110 | 178 | 182 | X | X | 165 | 166 | 147 | 148 | 160 | 162 | 115 | 115 | 93 | 93 |
| adjacent | | | | | | | | | | | | | | | | | | | |
| HGD | | | | | | | | | | | | | | | | | | | |
| UC with | | 99 | 100 | 109 | 110 | 178 | 182 | X | X | 165 | 166 | 147 | 148 | 160 | 162 | 115 | 115 | 93 | 93 |
| adjacent | | | | | | | | | | | | | | | | | | | |
| HGD | | | | | | | | | | | | | | | | | | | |

| Case | 32F | 32F | 52F | 52F | 36F | 36F | 21F | 21F | D17S250f | D17S250f | 78 | 78 | 58 | 58 | 64 | 64 |
|---|---|---|---|---|---|---|---|---|---|---|---|---|---|---|---|---|
| UC with | X | X | 180 | 184 | 114 | 114 | 160 | 161 | 158 | 158 | 181 | 181 | 113 | 114 | 150 | 150 |
| adjacent | | | | | | | | | | | | | | | | |
| HGD | | | | | | | | | | | | | | | | |
| UC with | X | X | 180 | 184 | 114 | 114 | X | X | X | X | 181 | 181 | 113 | 114 | 150 | 150 |
| adjacent | | | | | | | | | | | | | | | | |
| HGD | | | | | | | | | | | | | | | | |
| UC with | X | X | 180 | 184 | 114 | 114 | 160 | 159 | 158 | 158 | 181 | 181 | X | X | 150 | 150 |
| adjacent | | | | | | | | | | | | | | | | |
| HGD | | | | | | | | | | | | | | | | |
| UC with | X | X | 180 | 184 | 114 | 114 | 160 | 161 | 158 | 158 | 181 | 181 | X | X | 150 | 150 |
| adjacent | | | | | | | | | | | | | | | | |
| HGD | | | | | | | | | | | | | | | | |
| UC with | X | X | 180 | 184 | | | 160 | 161 | 158 | 158 | 181 | 181 | 113 | 114 | 150 | 150 |
| adjacent | | | | | | | | | | | | | | | | |
| HGD | | | | | | | | | | | | | | | | |
| UC with | X | X | 180 | 184 | | | 160 | 161 | X | X | 181 | 181 | X | X | 150 | 150 |
| adjacent | | | | | | | | | | | | | | | | |
| HGD | | | | | | | | | | | | | | | | |
| UC with | X | X | 180 | 180 | 114 | 114 | 160 | 160 | X | X | 181 | 181 | X | X | 150 | 150 |
| adjacent | | | | | | | | | | | | | | | | |
| HGD | | | | | | | | | | | | | | | | |
| UC with | X | X | 180 | 184 | 114 | 114 | X | X | 158 | 158 | 181 | 181 | 113 | 114 | 150 | 150 |
| adjacent | | | | | | | | | | | | | | | | |
| HGD | | | | | | | | | | | | | | | | |
| UC with | X | X | X | X | 114 | 114 | X | X | X | X | 181 | 181 | 113 | 114 | 150 | 150 |
| adjacent | | | | | | | | | | | | | | | | |
| HGD | | | | | | | | | | | | | | | | |
| UC with | X | X | 180 | 184 | 114 | 114 | X | X | 158 | 158 | 181 | 181 | 113 | 114 | 150 | 150 |
| adjacent | | | | | | | | | | | | | | | | |
| HGD | | | | | | | | | | | | | | | | |
| UC with | X | X | 180 | 184 | | | 160 | 161 | 158 | 158 | 181 | 181 | 113 | 114 | 150 | 150 |
| adjacent | | | | | | | | | | | | | | | | |
| HGD | | | | | | | | | | | | | | | | |
| UC with | X | X | 180 | 184 | | | 160 | 161 | 158 | 158 | 181 | 181 | 113 | 114 | 150 | 150 |
| adjacent | | | | | | | | | | | | | | | | |
| HGD | | | | | | | | | | | | | | | | |

| Case | D5S346f | D5S346f | 66F | 66F | 87F | 87F | 104F | 104F | 81F | 81F | 103F | 103F | 83F | 83F | BAT26f | BAT26f |
|---|---|---|---|---|---|---|---|---|---|---|---|---|---|---|---|---|
| UC with | 108 | 108 | 175 | 175 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | 175 | 175 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | 176 | 176 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | 175 | 175 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | 175 | 175 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | 175 | 175 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | 175 | 176 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | X | X | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | X | X | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | 175 | 176 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | 175 | 176 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |
| UC with | 108 | 108 | 175 | 176 | 201 | 203 | 126 | 127 | 155 | 155 | 172 | 172 | 205 | 205 | 118 | 118 |
| adjacent | | | | | | | | | | | | | | | | |
| HGD | | | | | | | | | | | | | | | | |

| Case | 103F | 103F | 102F | 102F | 107F | 107F | BAT25f | BAT25f | 83F | 83F | BAT26f | BAT26f | 103F | 103F | 102F | 102F |
|---|---|---|---|---|---|---|---|---|---|---|---|---|---|---|---|---|
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |
| UC with | 188 | 189 | 154 | 154 | 117 | 117 | 123 | 124 | 205 | 205 | 118 | 118 | 188 | 189 | 154 | 154 |
| adjacent | | | | | | | | | | | | | | | | |
| HGD | | | | | | | | | | | | | | | | |

| | | | | | Total | | | | Mutants |
|---|---|---|---|---|---|---|---|---|---|
| | | | | | positive | Total | Total | Total | per |
| Case | 107F | 107F | BAT25f | BAT25f | biopsies | epithelium | stroma | mutations | read |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | | | | | |
| adjacent | | | | | | | | | |
| HGD | | | | | | | | | |
| UC with | 117 | 117 | 123 | 124 | ⅚ | 5 | 6 | 11 | 3.54% |
| adjacent | | | | | | | | | |
| HGD | | | | | | | | | |

### Example 2

Studies were undertaken to determine whether there is variability in the performance of a biomarker based on 1) the physical distance of the biopsy tested from the neoplasia 2) the presence of inflammation in the biopsy, 3) the variability that may occur in between non-dysplastic biopsies within an individual case and 4) the number of sample biopsies per case that are optimum for an accurate biomarker reading.

To evaluate these variables biomarker studies were performed on multiple biopsies obtained throughout the individual colons of UC cases, herein referred to as “mapping studies”. An average of 7 biopsies were taken from along the full length of the individual colons of 10 UC Progressors, including samples of low-grade dysplasia (LGD), HGD, and at least 5 biopsies of non-dysplastic mucosa. Five biopsies were analyzed for each of 5 UC Non-progressors. The biopsies were separated into epithelial and stromal fractions by EDTA shake-off and run as independent samples. Every biopsy was annotated for its degree of inflammation (graded 1-4), location in the colon, distance to nearest dysplasia/cancer, and histology. Every case was annotated with regard to age, sex, duration of UC, and presence or absence of Primary Sclerosing Cholangitis (a liver disease that increases the risk of colon cancer).

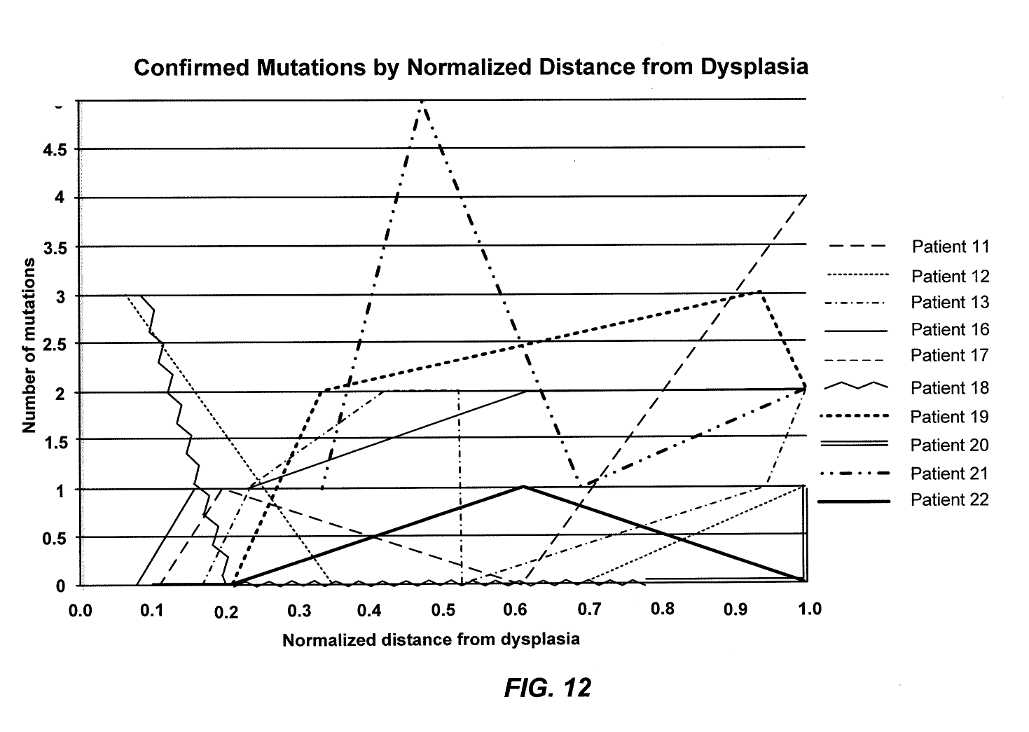

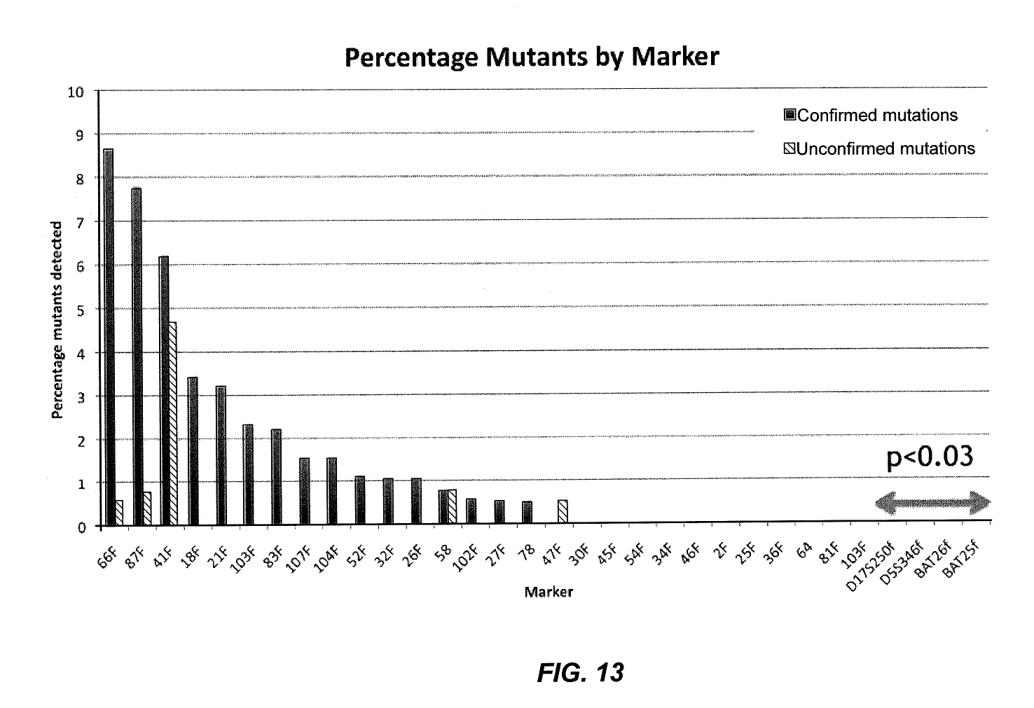

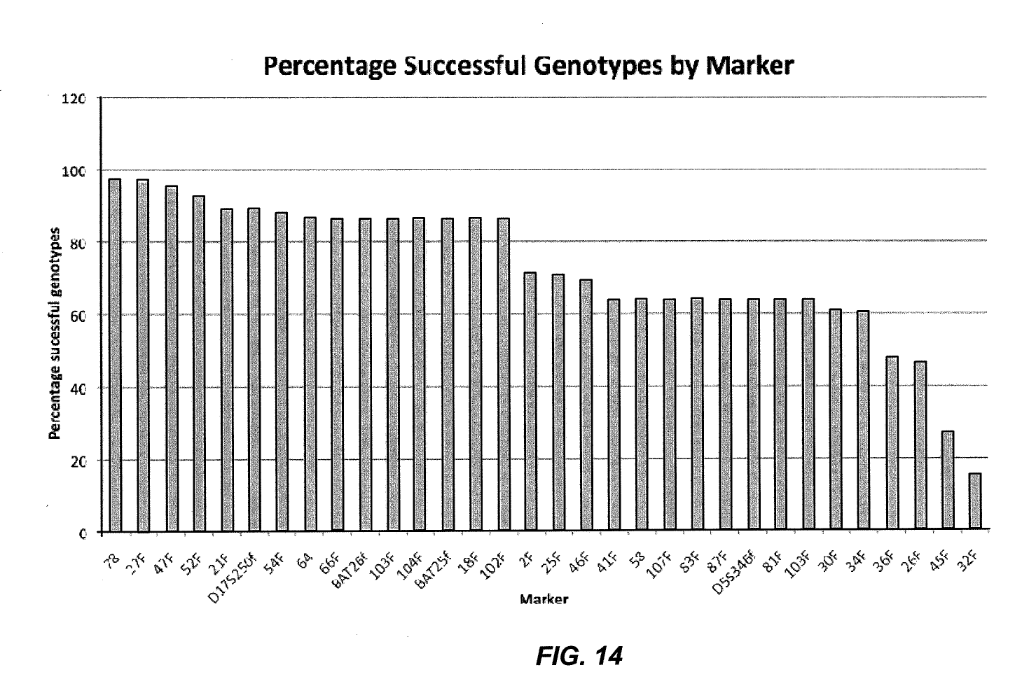

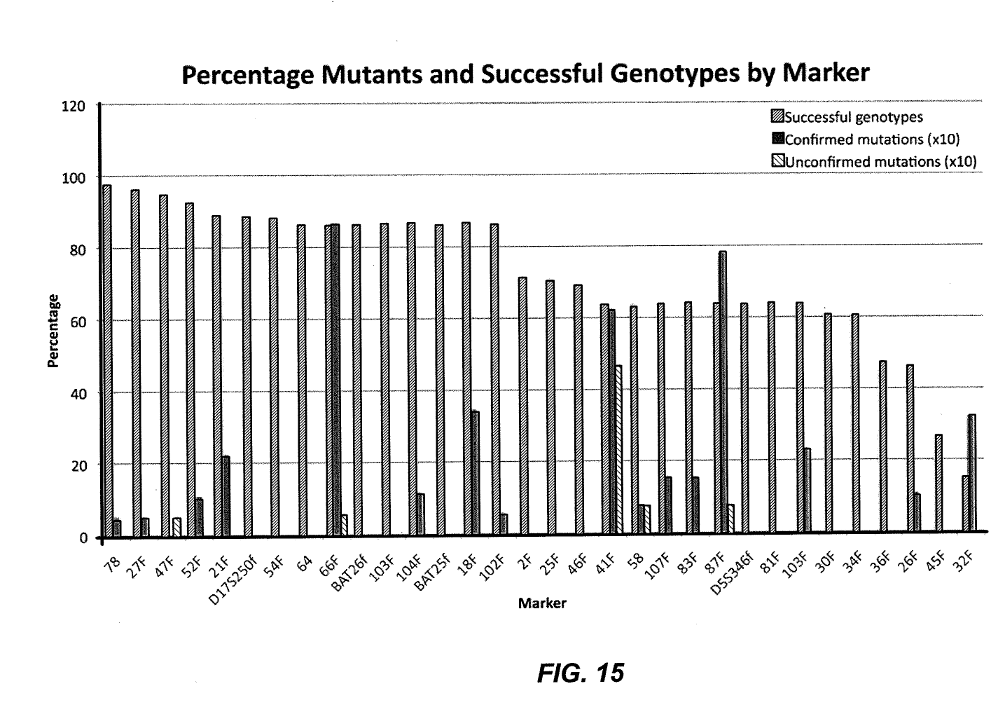

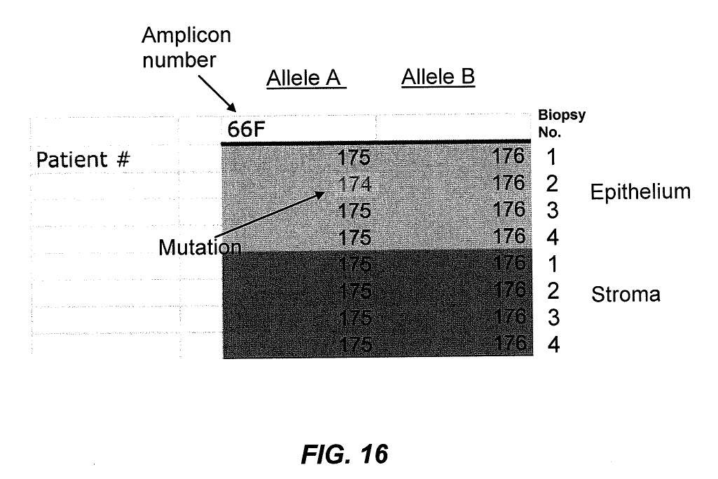

The mapping studies of variability and reproducibility have been completed for the poly-G assay. It was determined that the distance to dysplasia did not influence the poly-G slippage: cumulatively speaking, there were 26 mutations present in the non-dysplastic biopsies that were immediately adjacent to neoplasia, while 30 mutations were cumulatively present in the non-dysplastic biopsies that were most distant from dysplasia. Gender of the patient, duration of disease, or presence of inflammation had no influence on performance of the biomarker. Between four and five biopsies were required to reach the sensitivity of 100% and specificity of 93%. Thus, while all of the Progressors had at least 1 biopsy with mutations, the mapping studies do show that there is some variability in whether biopsies within a Progressors colon demonstrate measurable mutations using the current set of PCR primers. FIG. 12 illustrates that some Progressor patients have more non-dysplastic biopsies with mutations than do other Progressors and that the number of mutations does not show any relationship to distance from the site of dysplasia along the length of the colon.

The probability of obtaining a successful (readable) genotype was dependent on the specific poly-G tract being tested and is likely related to inherent polymorphic variability in the population (some individuals simply do not possess a portion of the sequences being amplified even in normal tissue) as well as PCR-specific issues. Also of note, the frequency of detectable slippage events is highly variable among the different poly-G tracts tested, e.g. it is likely that there are “hotspots” for poly-G slippage.

The poly-G assay is the most sensitive and least variable biomarker yet tested, as noted above, there was little variability in the marker based on location in the colon, proximity to neoplasia, or level of inflammation. The per sample labor and material costs are fairly low and there should be little difficulty in translation from a research protocol into a formal clinical assay since all components of the assay (PCR and capillary sequencing) have previously been granted FDA approval.

The present examples demonstrate that in UC progressors there are clonal expansion events present that are either not present, undetectable, or present at much lower frequency in UC non-progressors. Moreover, these clonal expansions are pathological by virtue of not being present in normal colon.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 3

### Polyguanine Tract Genotyping

Microsatellite genotyping by capillary electrophoresis produces an analog signal that reflects the predominant allele lengths within a DNA sample. We have previously identified somatic mutations in polyguanine alleles that are unique to single mouse cells by clonally expanding their genome in vitro prior to analysis (Salipante SJ & Horwitz M S, Proc Natl Acad Sci USA (2006) 103(14):5448-5453; Salipante SJ, E M, & Horwitz M S (Phylogenetic Analysis of Developmental and Postnatal Mouse Lineages. (Submitted to Evolution and Development) each of which is hereby incorporated by reference in their entirety for all purposes). Such mutations cannot be detected in bulk-tissue because rare alleles are obscured by more prevalent ones. Initial experiments in the present study sought to establish the feasibility of detecting novel polyguanine genotypes in the human colon where clonal expansion has occurred in vivo.

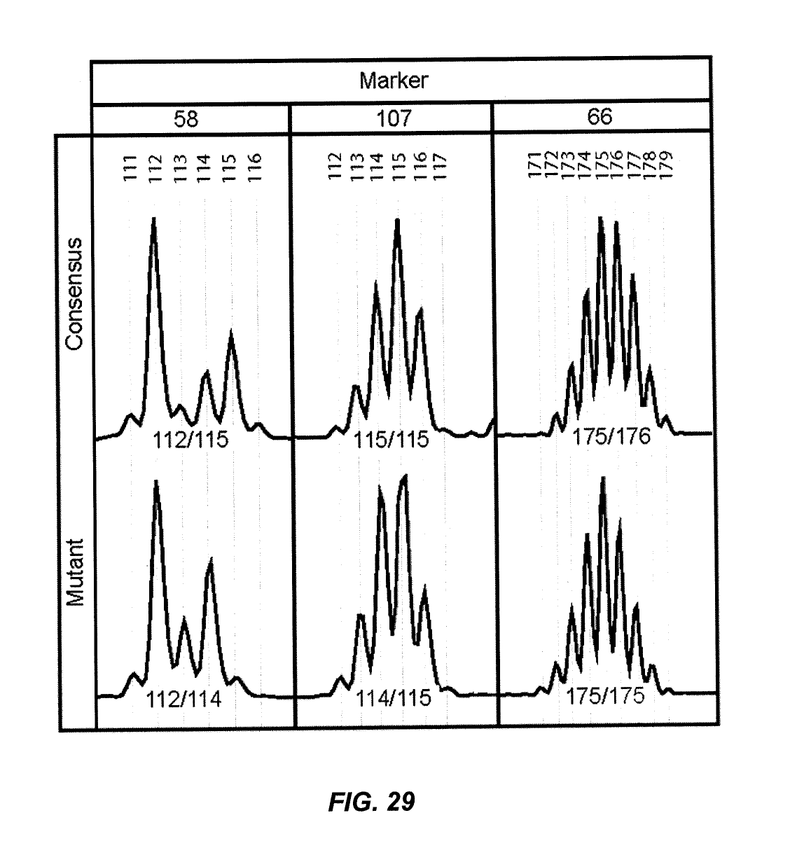

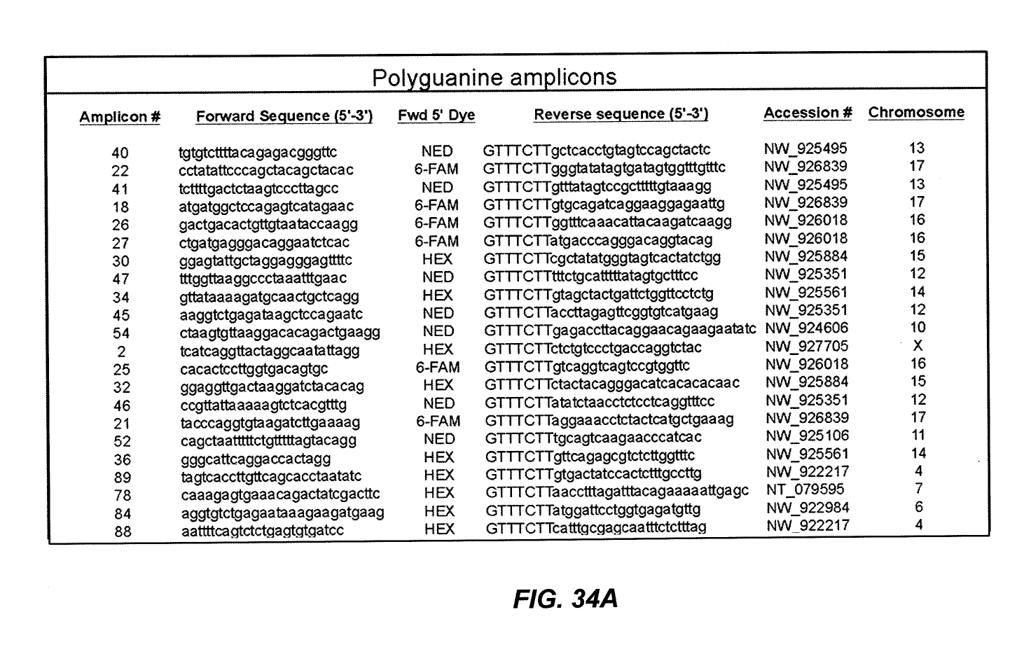

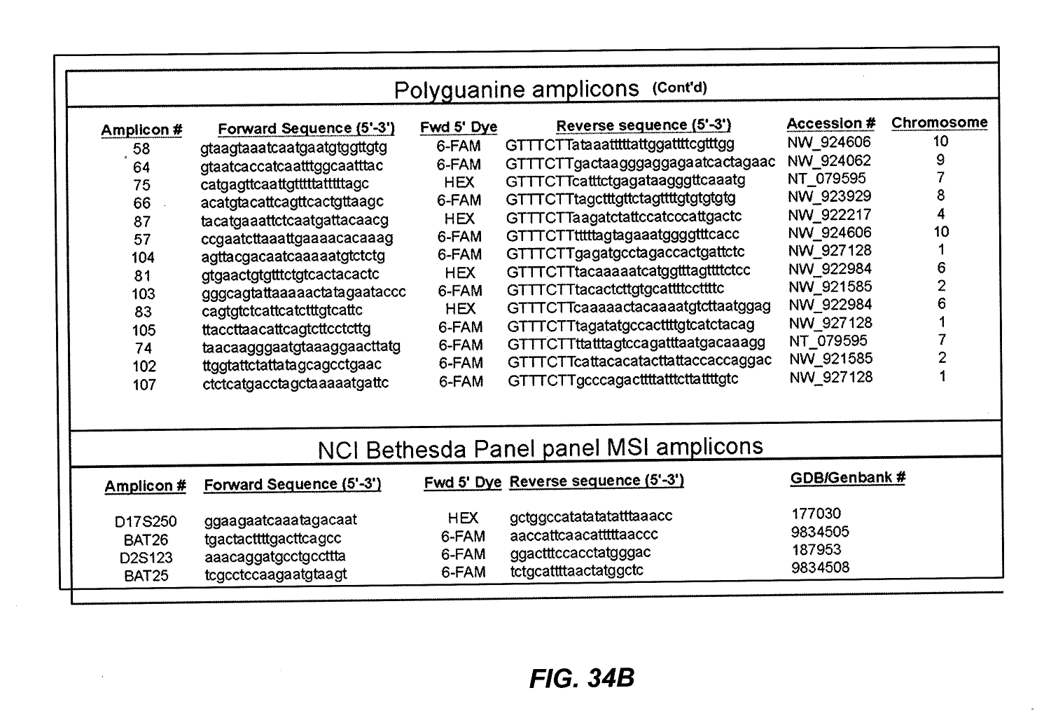

Fluorescently labeled PCR primers were designed to flank 36 intergenic and intronic polyguanine tracts, arbitrarily selected from throughout the human genome (FIGS. 34A and 34B). The amplified product of each marker displayed either one or two maximal peaks depending on whether the individual genotyped was heterozygous or homozygous for repeat length at the locus (Salipante SJ & Horwitz M S, Proc Natl Acad Sci USA (2006) 103(14):5448-5453) (FIG. 29). Adjacent sub-maximal peaks (“stutter”) are an artifact of PCR amplification resulting from strand slippage that generates insertions and deletions in a subset of amplicon molecules (Clarke L A et al., MP, Mol Pathol (2001) 54(5):351-353). To confirm reproducibility of genotyping based on maximal peak position, a single DNA sample was used to initiate 15 replicate PCRs for each primer set. Genotype assignments made by a blinded observer were 100% concordant among replicates.

To determine the threshold for detection of mutants within a heterogeneous population of mutants and adjacent non-mutant cells, mixing experiments were carried out. For a subset of markers, DNA from two individuals whose germline genotypes differed by a single base-pair in one allele were combined in ratios of 20% increments. Electropherograms from these mixtures were then compared to those from the original, unmixed DNAs. The fractional abundance required to reliably identify a simulated mutant clone varied by marker but consistently fell between 40-60%. Thus, identification of a mutant allele by this technique indicates that a minimum of 40% of harvested cells must share the mutation.

### Clonal Expansions in Nondysplastic UC

We next investigated whether polyguanine tract mutations could be used to identify clonally expanded cell populations within UC tissue. Between 44 and 144 biopsies were harvested in an evenly spaced grid along the colon of 19 UC patients (15 after colectomy, 4 during colonoscopy). Eight individuals with no histological evidence of high-grade dysplasia (HGD) or cancer anywhere in the colon were classified as “Non-Progressors”. Eleven individuals with at least one biopsy with HGD or cancer were classified as “Progressors”. An average of 4.6 non-dysplastic biopsies were arbitrarily selected (average spacing of 20.1 cm) along each colon for genotyping. Of exception were three Non-Progressor and one Progressor cases where genotyped biopsies were limited to the rectum. DNA separately purified from epithelial and stromal cell fractions of a ˜9 mm² portion of each biopsy was genotyped at 28 polyguanine sites and four “Bethesda Panel” markers used to diagnose microsatellite instability (MSI) in DNA mismatch repair-deficient states (Boland C R, et al. Cancer Res (1998) 58(22):5248-5257). Personnel performing the genotyping were blinded to all clinical information.

Within each patient, the majority of genotypes for a given marker were identical across all samples. Occasionally, however, the allele pattern of a biopsy tissue fraction differed from the predominant, “consensus genotype” identified elsewhere in the colon, indicating that a rare, clonally expanded mutation had been sampled (FIG. 29). A complete listing of genotype calls is provided in supplemental FIG. 2 of Salk et al. 2009 (Salk JJ et al., Proc Natl Acad Sci USA (2009) November 19, incorporated herein by reference in its entirety for all purposes). This supplemental figure provides complete genotyping dataset. Horizontal boxes represent individual cases grouped by disease status: UC Non-Progressor (yellow), UC Progressor (red). Biopsies were separated into epithelial and stromal fractions and genotyped separately. Rows within each case indicate specific fractions of individual biopsies. Relevant clinical data for each individual/biopsy is given. Vertical columns making up the bulk of the table indicate the length of alleles for each polyguanine marker. The four rightmost marker columns (pink headings) correspond to non-polyguanine Bethesda Panel MSI markers. “X” indicates unsuccessful genotyping. Genotypes differing from the consensus genotype for that individual are shown in red. Markers where at least one mutant genotype was identified in an individual are highlighted: epithelial samples shown in violet, stromal samples in blue. Summary data by case and by marker are listed at the far right and bottom, respectively. Genotyping was performed under fully blinded conditions.

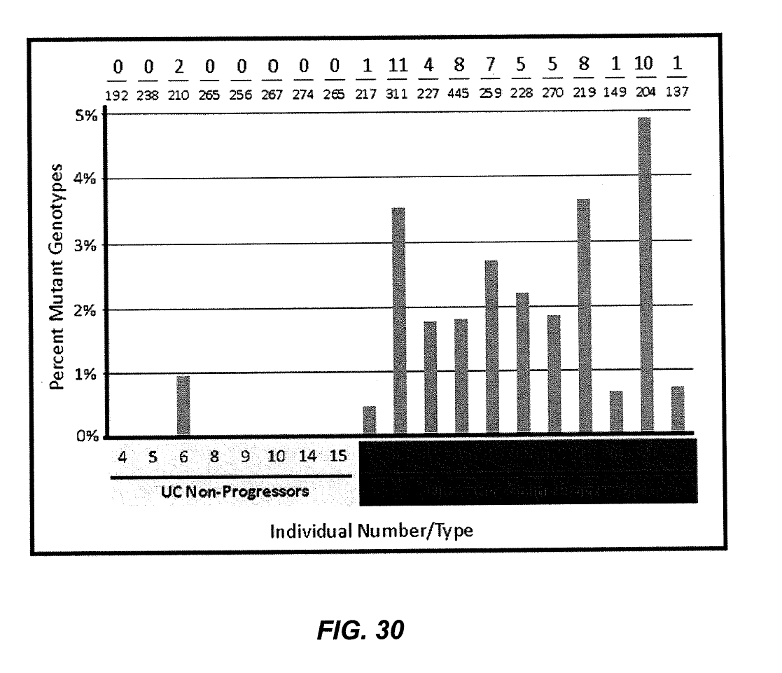

The presence of detectable clones was strongly correlated with progression status (FIG. 30). Of the 63 mutations identified, 97% occurred among Progressors. Whereas 100% of Progressors had at least one identifiable mutation, only one out of eight (12.5%) Non-Progressors showed any mutations (p<0.001, one-tailed Fisher's exact test). Although the overall prevalence of detectable mutations was low (˜1.4% of all successful genotypings), within the Progressor group, 63% of biopsies carried at least one mutant marker. Thus, at least two thirds of the non-dysplastic biopsies obtained from Progressor colon arose from post-zygotic clonal expansions. As a screening tool for detecting HGD or cancer elsewhere in the colon, identification of one or more mutant markers in non-dysplastic tissue was 100% sensitive and 88% specific.

Of the clonal mutations identified, 78% (49/63) occurred in biopsy epithelium while 22% (14/63) were found in biopsy stroma. In the majority of cases (94%), a matching mutation from the epithelium was not found in the corresponding stroma. Besides affirming the robustness of tissue separation, this finding suggests that clonal expansions infrequently cross compartment boundaries. Interestingly, of the two instances of a dual compartment mutation, one pair represented the only mutation found in the Non-Progressor group (patient 7) and might represent an embryonically-derived clonal lineage.

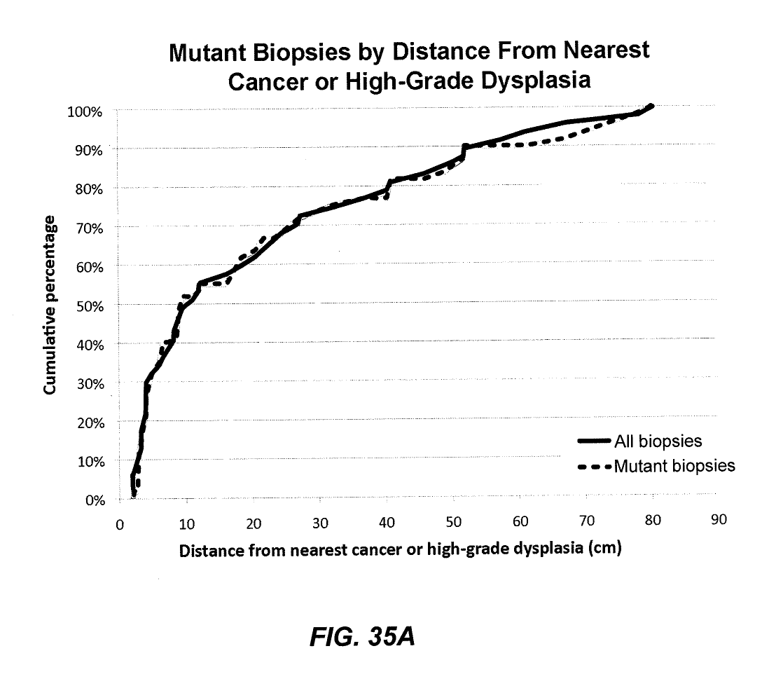

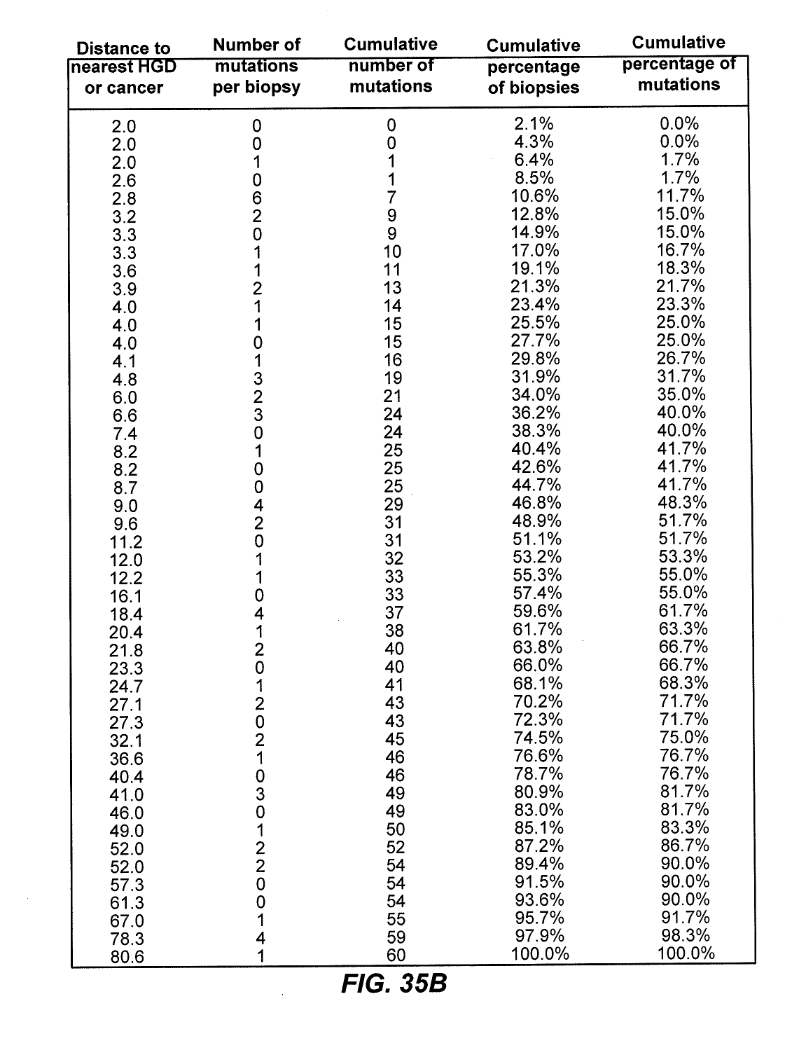

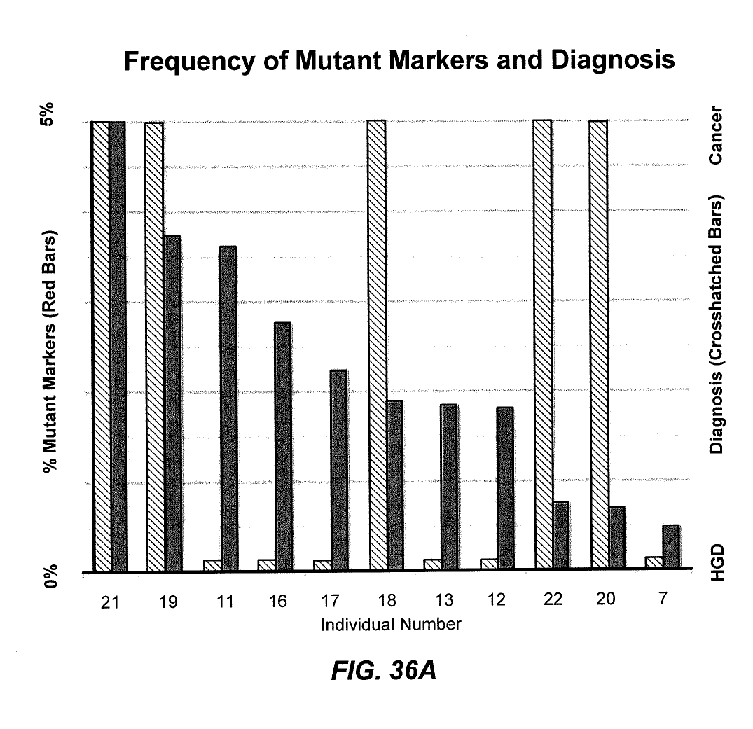

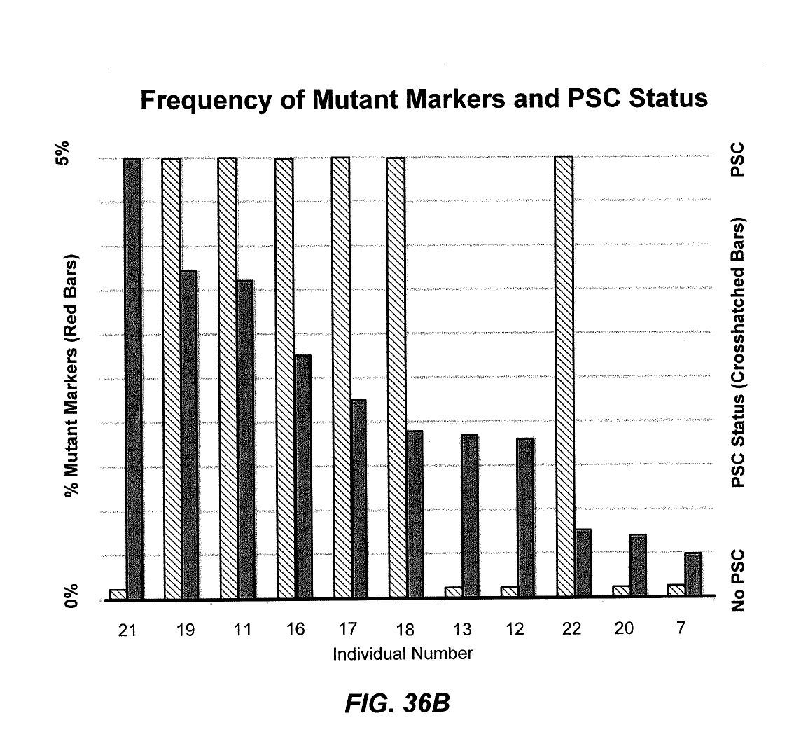

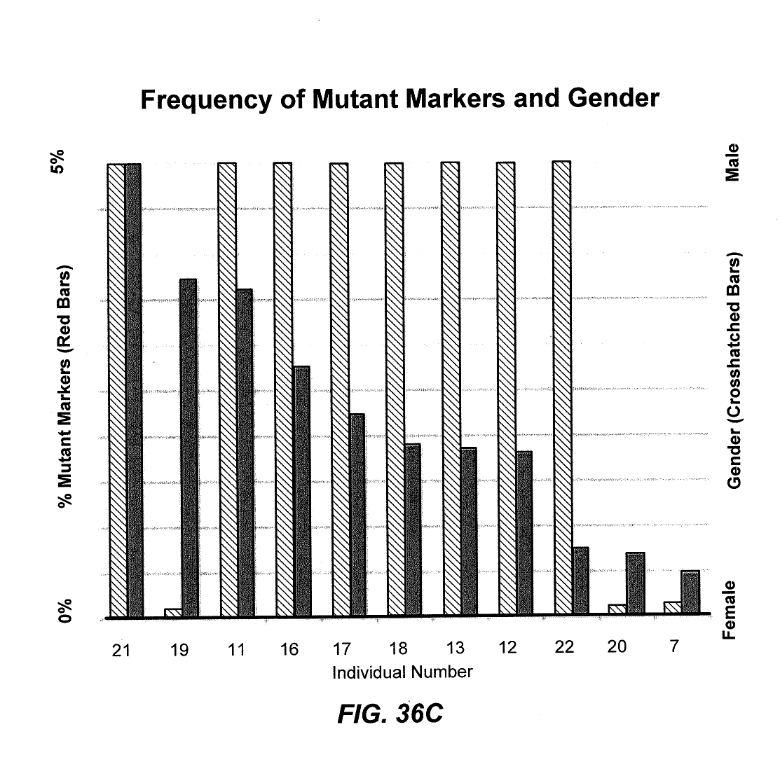

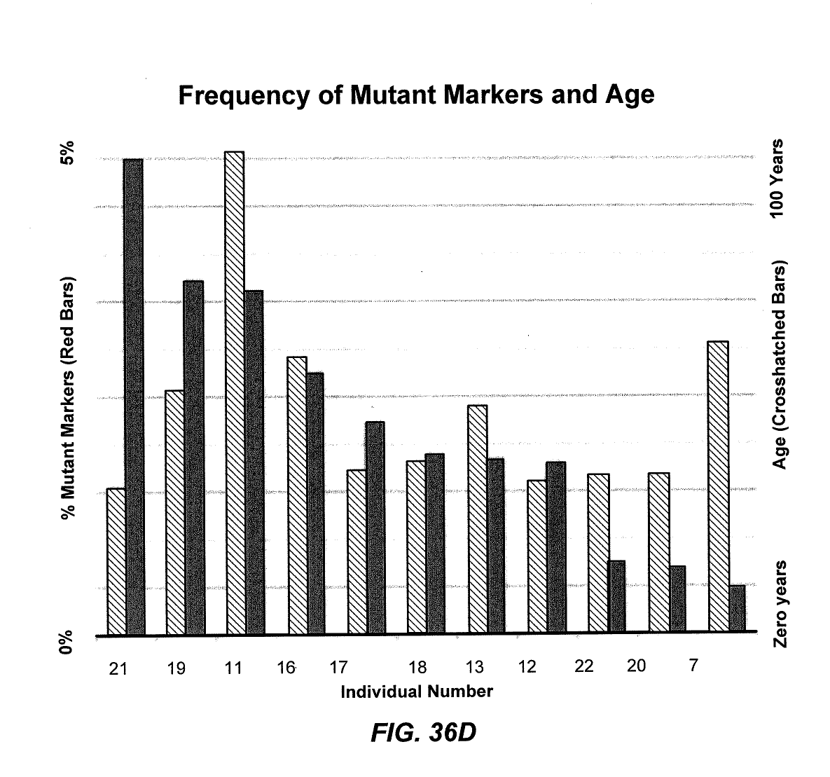

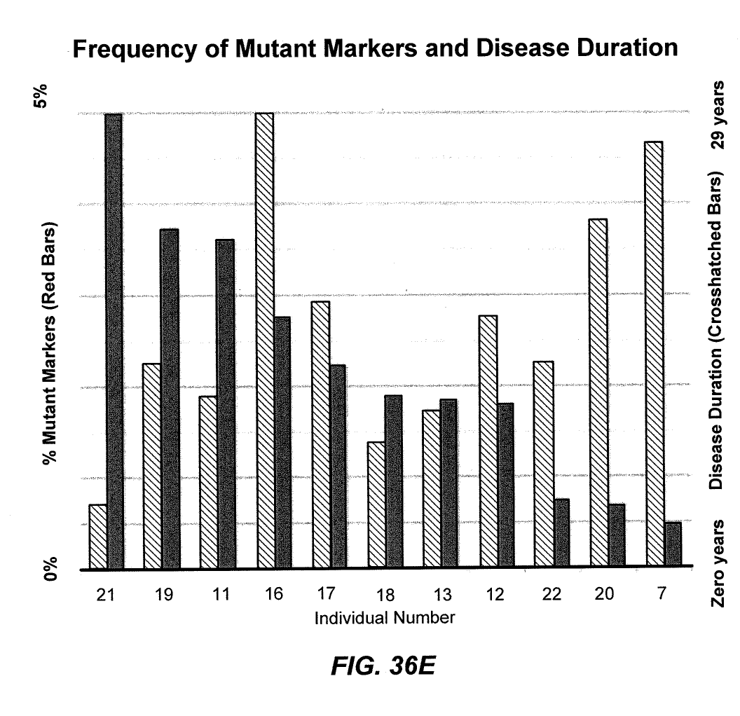

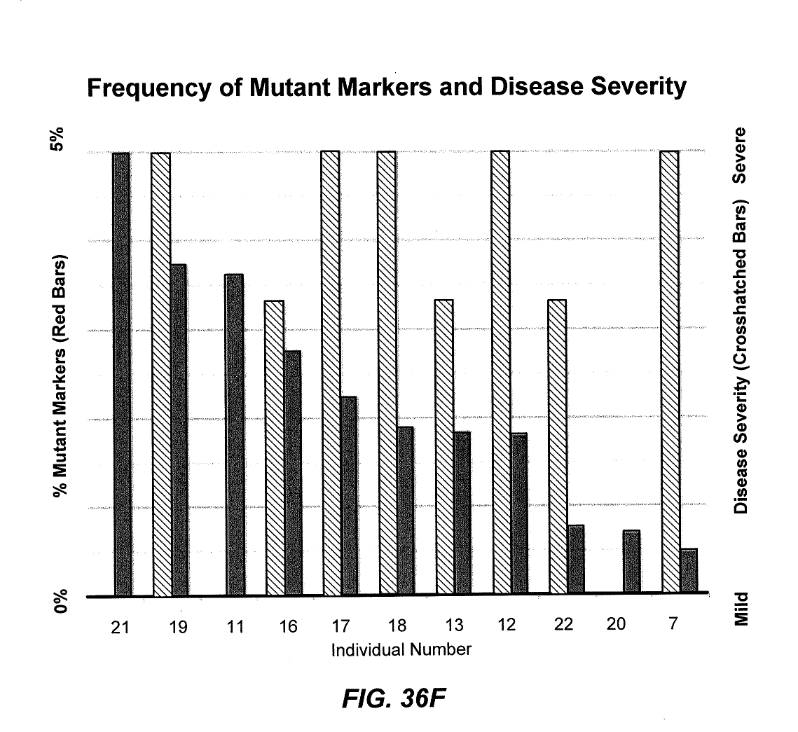

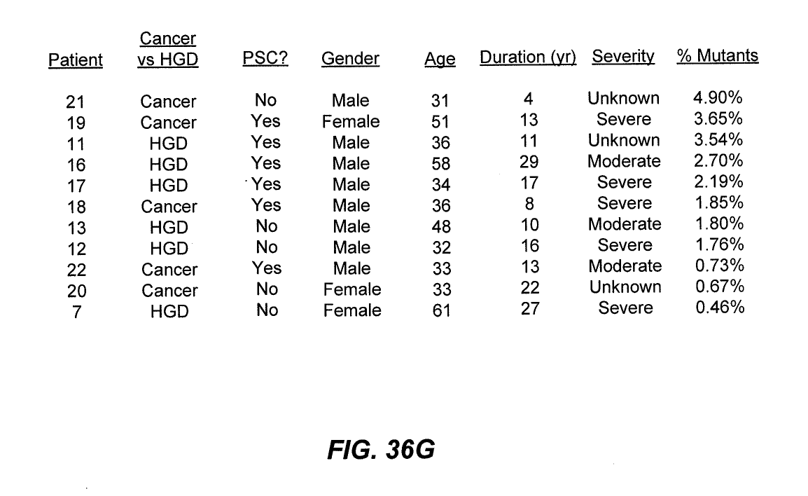

Of biopsies with a detectable clone, the majority (69%) were identified by a mutation in a single marker. In one third of mutant biopsies, however, from 2-6 markers were simultaneously altered. Based on an evolutionary model of cancer, clones having accumulated multiple independent mutations might have undergone more rounds of selection and expansion than clones with single mutations and potentially be more “cancer-like”. The number of mutations identified among all progressors was plotted as a cumulative function of distance to the nearest site of cancer or HGD (FIG. 35). The presence of detectable clones was independent of proximity to neoplasia. No significant associations (by Wilcox Rank Sum test) were found between the frequency of clonal mutations and age (p=0.73, dichotomized by median), gender (p=0.31), presence of primary sclerosing cholangitis [a positive modifier of cancer risk in UC] (p=0.20), duration (p=0.14, dichotomized by median) or clinical severity of UC (p=0.72), or whether progression was to HGD or cancer (p=0.72) (FIGS. 36A-G). The limited number of Progressors in this pilot study, however, is insufficient (estimated power of 7% at 0.05 alpha level) to conclude that a difference between observed mean mutation frequencies existed between different subgroups of Progressors.

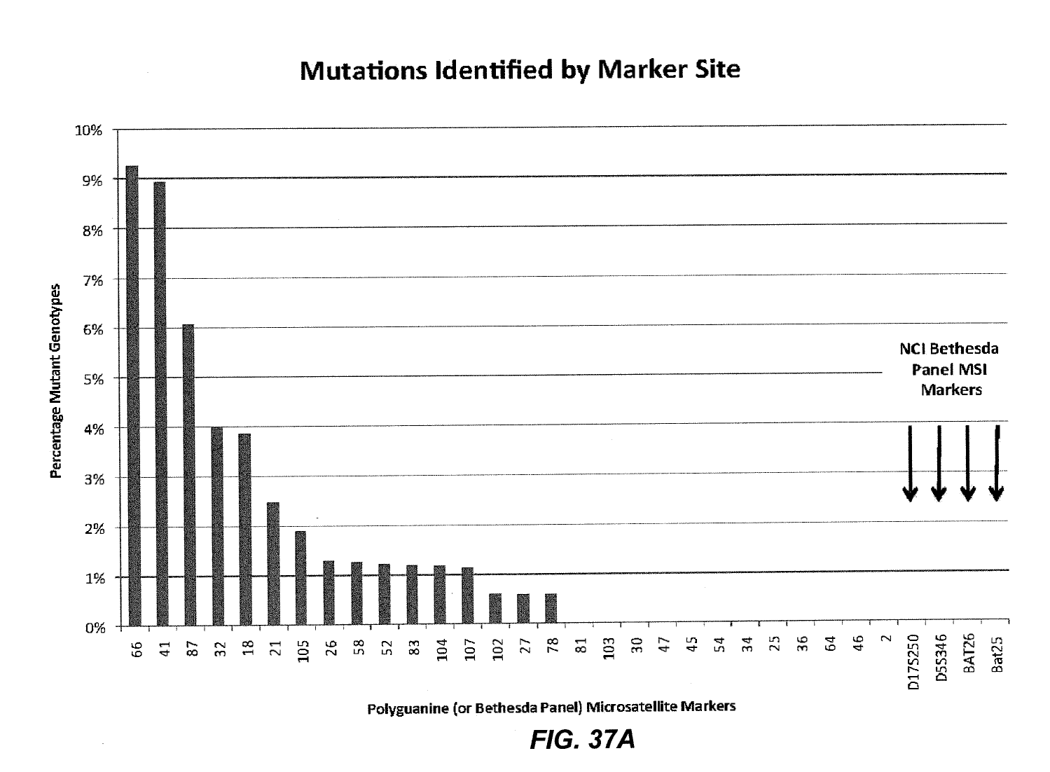

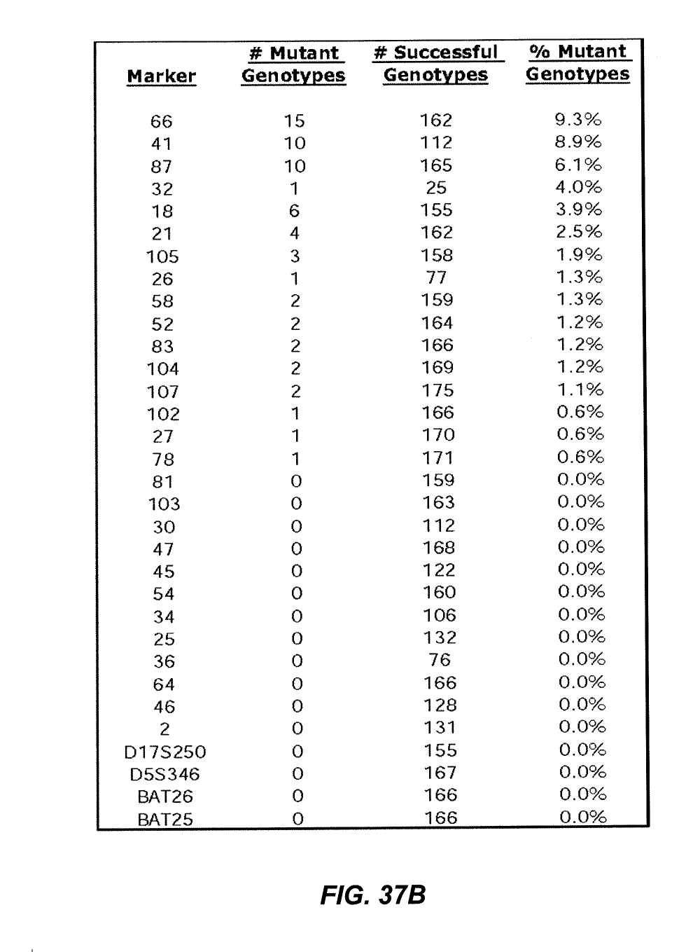

Mutations were found in some marker sites more frequently than in others (FIG. 37). No alterations were identified in half of the polyguanine sites. Significantly, no mutations were detected for any of the four Bethesda panel MSI markers tested indicating that polyguanine marker mutations are independent of DNA mismatch repair deficiency. In contrast, several other polyguanine loci were mutated in about 9% of Progressor samples. This non-normal distribution (p<0.001, Shapiro-Wilk normality test) likely reflects differences in slippage frequency inherent to particular loci (Salipante SJ, E M, & Horwitz M S (Phylogenetic Analysis of Developmental and Postnatal Mouse Lineages. (Submitted to Evolution and Development); Eckert K A & Hile S E, Mol. Carcinog. (2009) 48(4):379-388)).

Different types of genotype alterations were observed among mutant clones (FIG. 29; supplemental FIG. 2 of Salk et al. 2009). Deletions (51) were about four times more frequent than additions (12) among allele changes. Among mutations, 24 homozygote-to-heterozygote and 11 heterozygote-to-heterozygote alterations resulted from slippage of one allele. Twenty-three apparent heterozygote-to-homozygote mutations could have resulted either from allele slippage in the polyguanine tract or a chromosomal loss-of-heterozygosity (LOH) event, yielding an identical-appearing hemizygote. However, the latter explanation appears unlikely. The strong bias toward deletions was present in heterozygote-to-homozygotes (21 deletions, 2 additions) to the same degree as in the other two categories. If LOH predominated as a mechanism, the relative size of the allele lost would be determined randomly, yet this was not the case (p<0.001 against the null hypothesis that the large and small alleles disappear at equal frequency). Thus, most, if not all mutations observed resulted from slippage in a polyguanine tract with retention of both alleles prior to clonal expansion.

### Clonal Patch Size

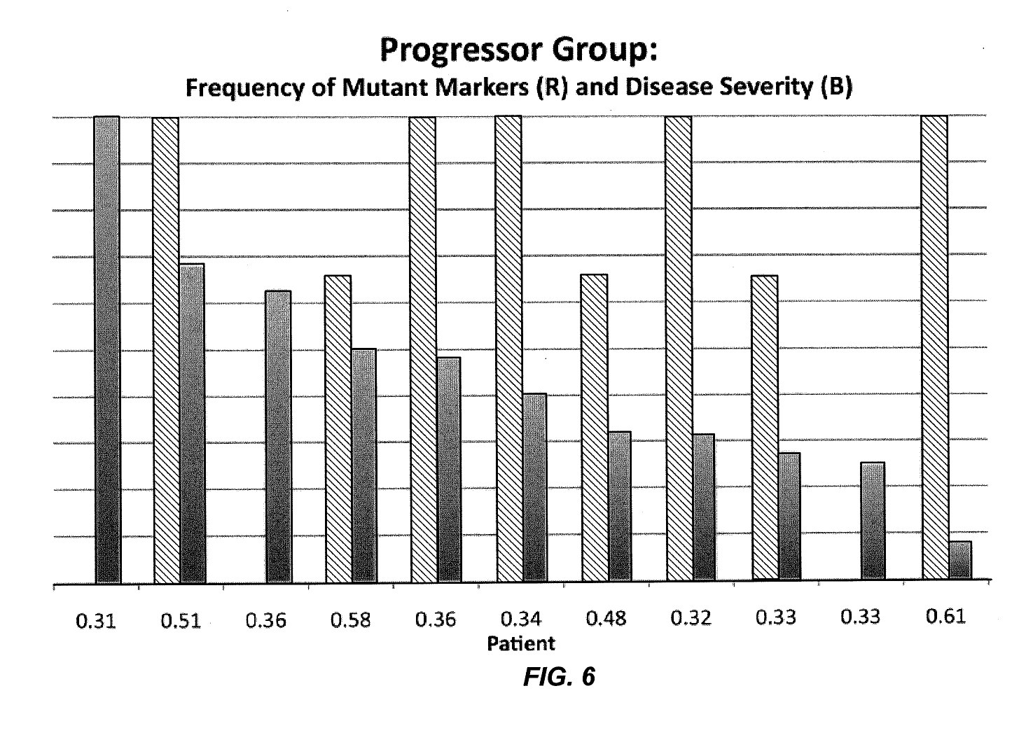

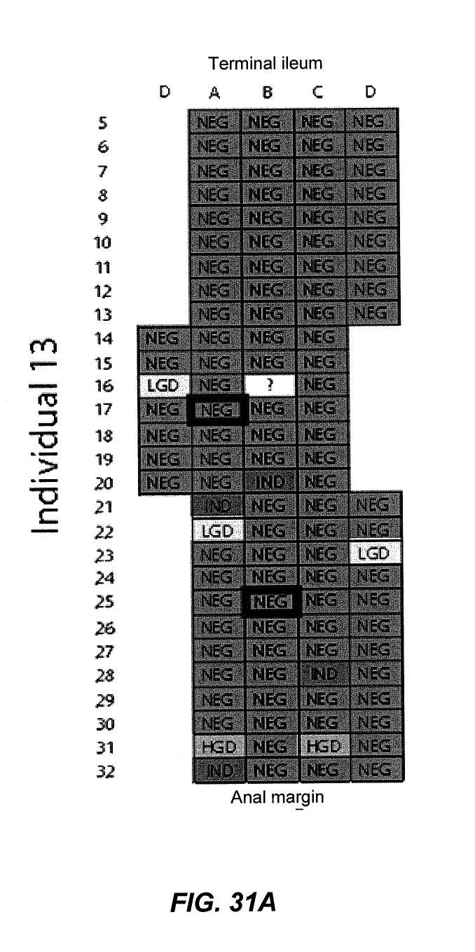

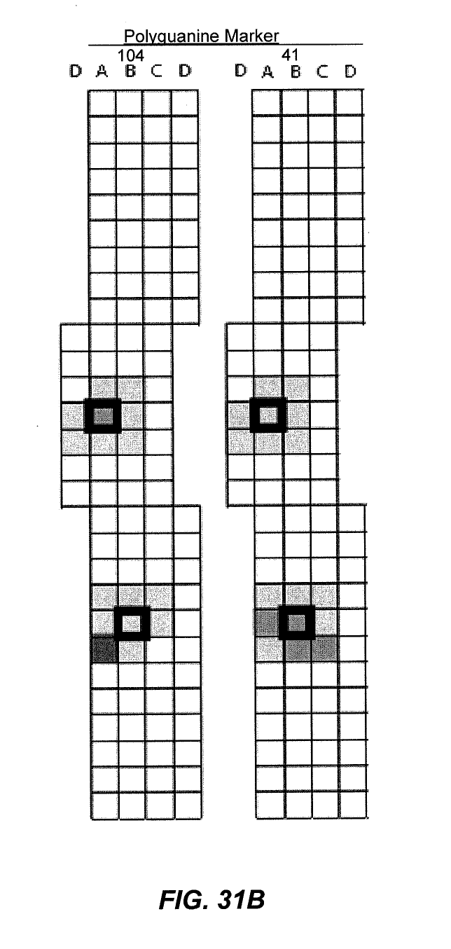

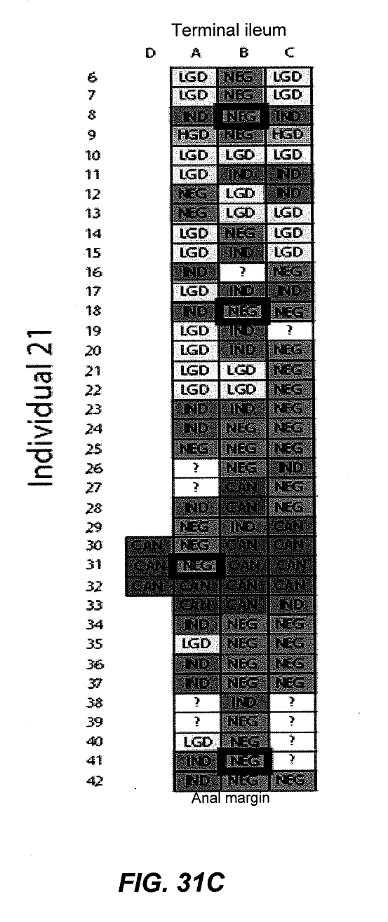

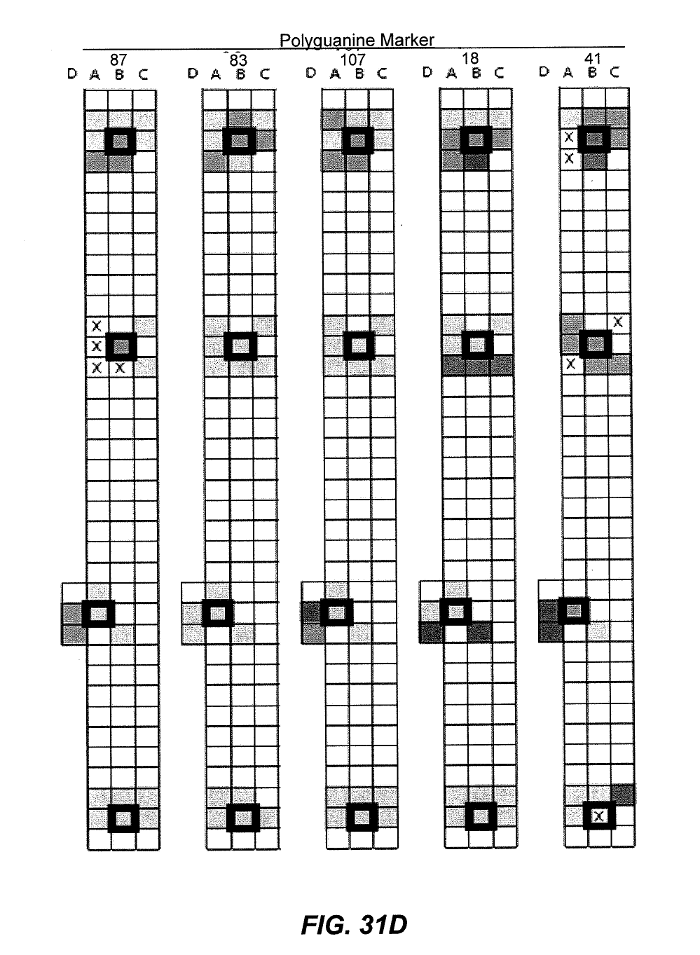

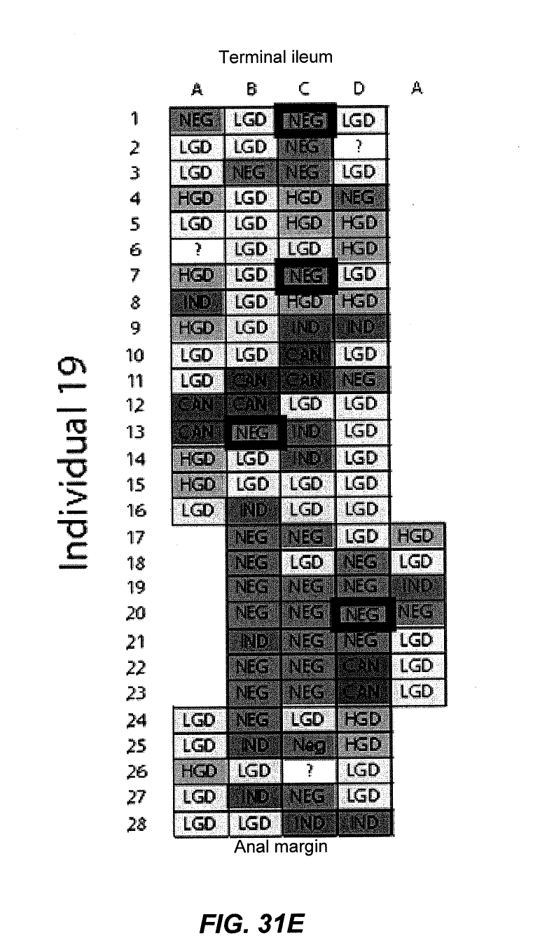

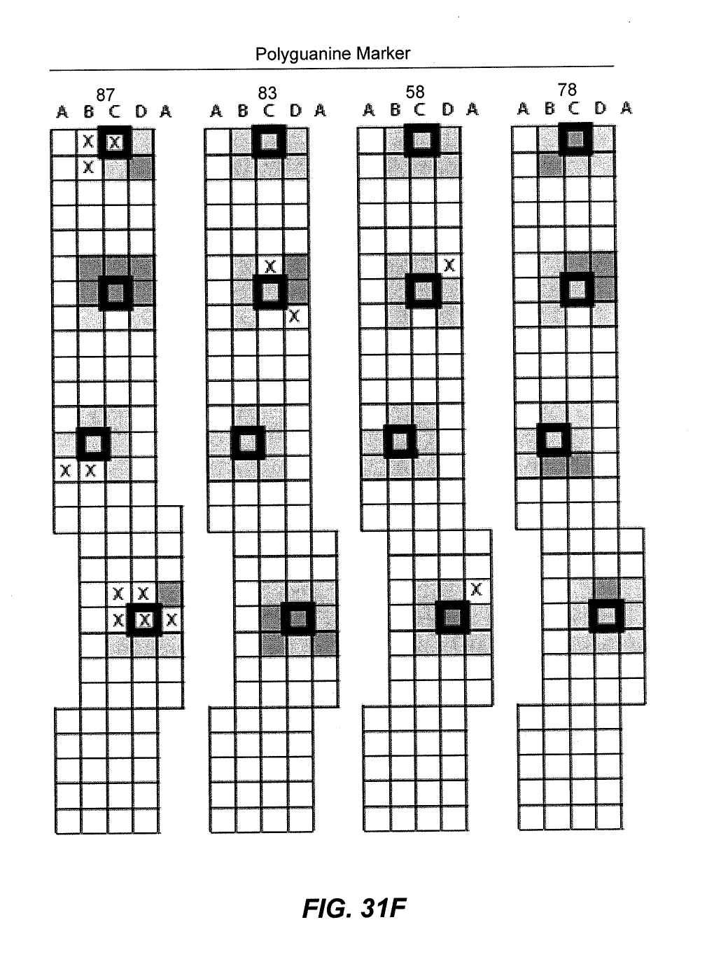

On six occasions, identical mutations were detected in two or more well separated biopsies from the same patient raising the question of whether a clonal patch extends over more than one biopsy site. To further characterize the spatial extent of clonal expansions, ten biopsy sites were selected from three patients, as regions for further analysis. Epithelial DNA was isolated from portions of 4-8 additional biopsies immediately surrounding each of the ten sites. Surrounding biopsies were of all histological subtypes and ranged from 1 to 5 cm from the central biopsy. As a control, epithelial DNA was also re-extracted from the ten original samples. All samples were then genotyped at a subset of markers previously identified as mutant in one or more of the central biopsies for each individual. FIGS. 31A-F graphically summarizes the spatial relationship between mutations identified in individuals 19, 21, and 13. Complete genotype information is provided in supplemental FIG. 6 of Salk et al. 2009.

Supplemental FIG. 6 of Salk et al. 2009 provides complete data for clonal patches identified by polyguanine mapping. Epithelium from biopsies surrounding each of ten samples used in the initial study were genotyped at markers sites previously found to be mutant in a subset of ten samples used in the initial study (outlined boxes). Longitudinally opened colectomy specimens from three individuals are diagrammed separately. Colectomy diagrams are divided into boxes representing individual biopsies measuring ˜9 mm², taken at evenly spaced intervals within an alphanumeric grid. The histological diagnosis from each biopsy is indicated at left: NEG, negative for dysplasia; IND, indefinite for dysplasia; LGD, low grade dysplasia; HGD, high grade dysplasia; CAN, cancer; ?, no data. The genotypes of biopsies for specific polyguanine markers are indicated at right. Empty boxes represent biopsies not genotyped, “X” indicates unsuccessful genotyping. Grey fields indicate biopsies with the consensus genotype for the marker, and different colors represent distinct mutant genotypes within an individual. Clustering of identical mutant genotypes in adjacent biopsies suggests large, clonally-derived patches. Within each panel, the previously determined genotypes of the initial biopsies are listed to the right. Genotypes of surrounding biopsies and repeat genotypes of central biopsies are shown. Repeat testing of central biopsies uniformly obtained the same genotype as previously identified.

Blinded re-genotyping of central samples was 100% concordant with initial results. A large number of mutations were identified in surrounding biopsies. Many of these were identical to those found in the central biopsies, strongly suggesting derivation from a common clonal “patch”. In total, 30 clones were identified among these three patients, of which 18 encompassed two or more adjacent biopsies. In some instances, patches were small; the mutant genotype of a central sample was completely surrounded by biopsies bearing consensus genotypes at the same marker (i.e., FIGS. 31E-F: Individual 13, biopsy 17A). In other cases patches were large, extending into multiple biopsies and probably beyond (i.e., FIGS. 31A-B Individual 19, rows 6-7). Up to four different mutant genotypes were seen in the same marker within a single individual (i.e., FIGS. 31B-C: Individual 21, marker 18). Spatial clustering of discrete variants reinforces the technical precision of genotyping and indicates independent expansion events.

Clonal patches were identified among biopsies of all histological diagnoses. Some large patches were found in exclusively nondysplastic areas (i.e., FIGS. 31E-F: Individual 13, rows 25-26, marker 41), whereas other patches were present in neoplastic mucosa of a variety of grades (i.e., FIGS. 31A-B: Individual 19, rows 6-7, marker 87). Thus, genetically-defined cell lineage mapping identifies clones that cannot be discerned from morphologic characteristics. In some instances, adjacent clonal patches defined by different mutant markers could be clearly distinguished from each other (i.e., FIGS. 31E-F: Individual 13, rows 25-26). In other cases, a patch defined by one marker appeared within a larger patch defined by a different marker (i.e., FIGS. 31A-B: Individual 19, rows 6-7). Such serially-nested clones provide information regarding the phylogenetic history of expansion. Within individual 19 for example, parsimonious logic dictates that the three biopsy patch defined by marker 78 most likely arose as a subclone of the six biopsy patch defined by marker 87.

### Complete Colon Map

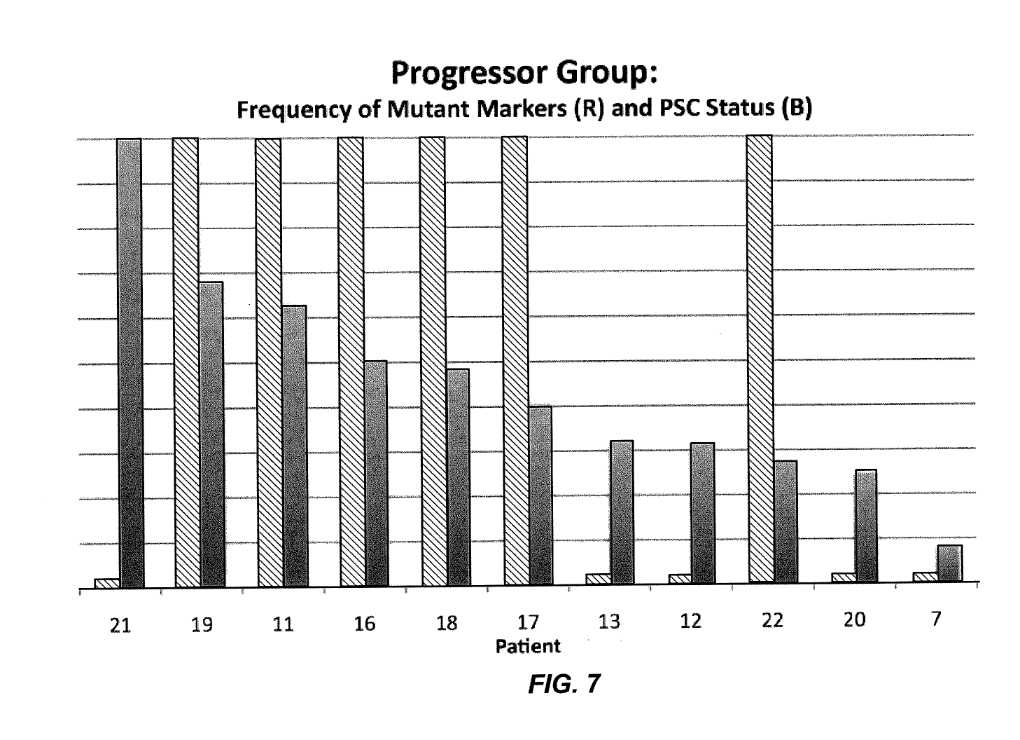

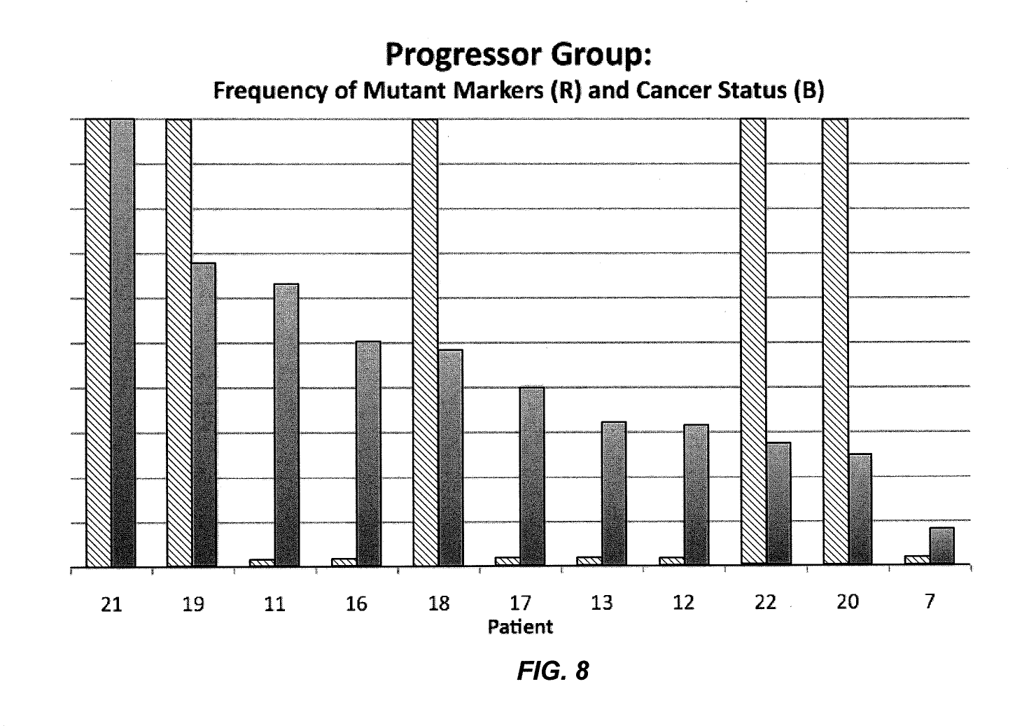

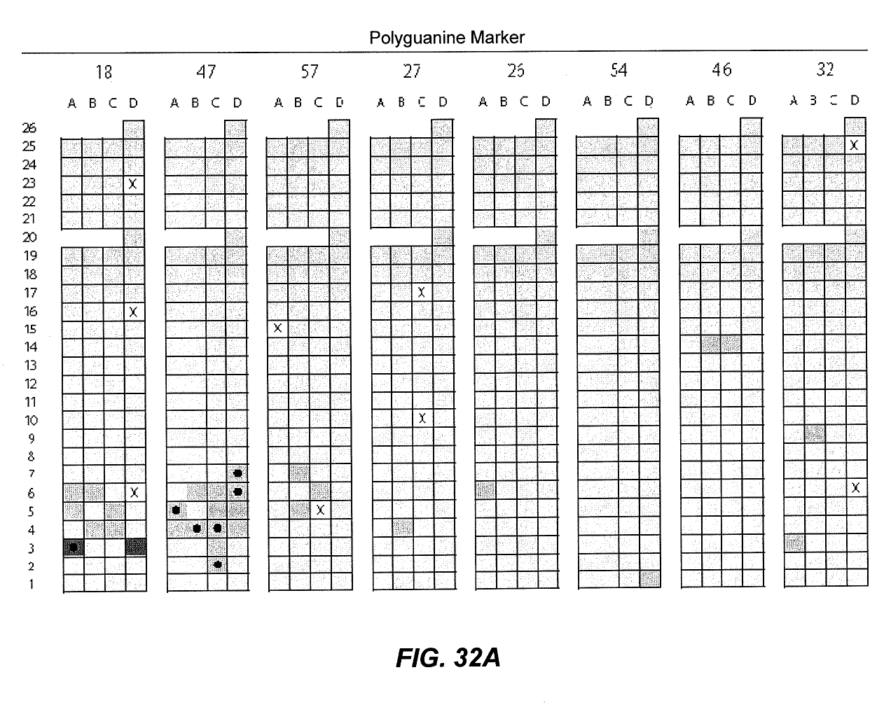

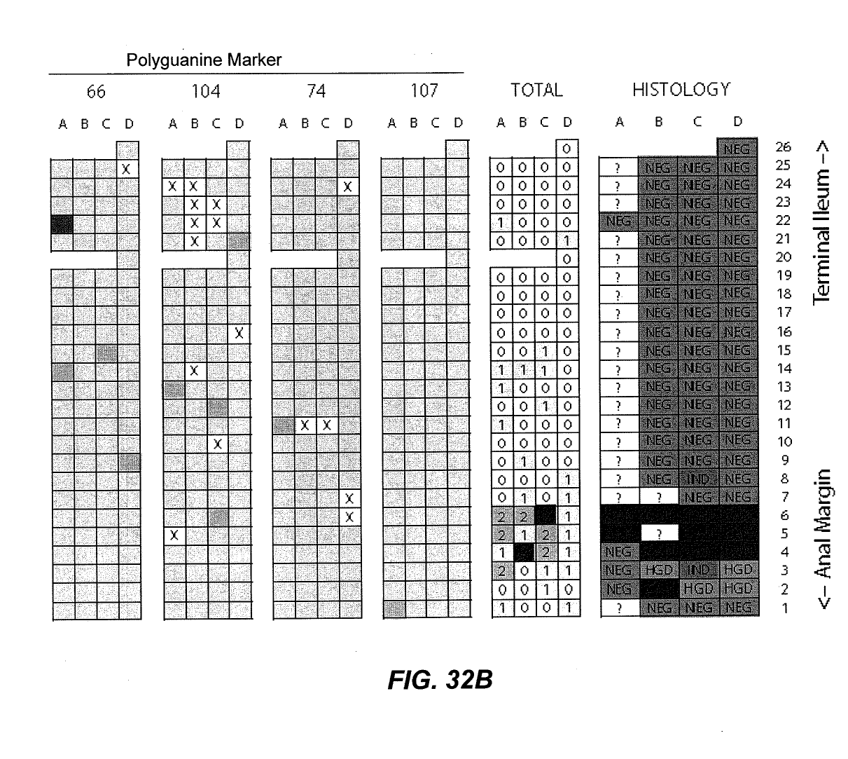

The extent of dysplasia in Progressor colons varied from limited (i.e., Individual 13) to widespread (i.e., Individuals 19 and 21). To determine the extent of clonal expansions in a best approximation of “early” UC Progressor tissue, we densely sampled an additional colectomy specimen with pancolonic inflammatory disease where histological changes were limited to the rectosigmoid colon. Epithelial DNA was isolated from a grid of 98 biopsies evenly spaced along the entire colon and blindly genotyped using the complete panel of polyguanine markers (FIGS. 32A-B, complete genotype data in supplemental FIG. 7 of Salk et al. 2009).

Supplemental FIG. 7 of Salk et al. 2009 provides a full data set from polyguanine mapping of a complete UC Progressor colon. A longitudinally opened colectomy specimen is diagramed with small boxes representing evenly spaced biopsies measuring ˜9 mm² within an alphanumeric grid. The histological diagnosis of each biopsy is indicated at far right: NEG, negative for dysplasia; IND, indefinite dysplasia; HGD, high grade dysplasia; CAN, cancer; ?, No data. Biopsy genotypes for various polyguanine marker are indicated in separate grids. “X” indicates unsuccessful genotyping. Grey fields indicate biopsies with the consensus genotype for the marker, and different colors represent distinct mutant genotypes. Underlined genotypes indicate biopsies where a mixture of consensus and mutant genotypes were observed, suggesting a mixed population of cells with different genotypes. The total number of mutant genotypes identified across all markers is reported for each biopsy (heat map). Large, clonally-derived patches identified by three markers were observed near the cancer site. Numerous smaller patches were detected throughout the non-dysplastic portions of the colon.

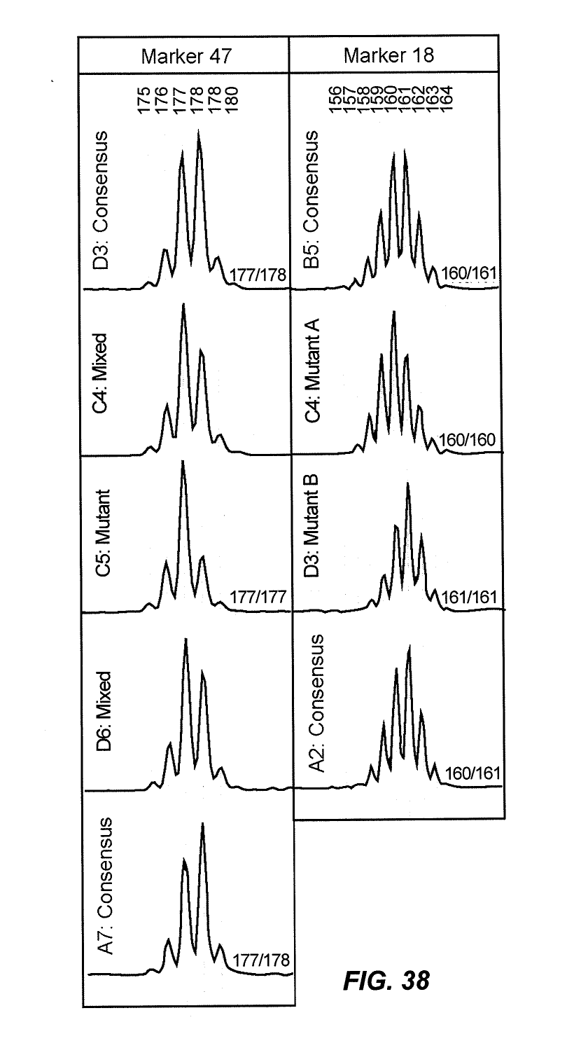

Mutations were identified in nearly one third (31/98) of biopsies tested. The majority of clones detected in the histologically negative portion of the colon were small in size, with mutant genotypes appearing only in single biopsies. At the cancer focus, however, a large clonal patch extending over at least 13 biopsy sites was found. The abundance of mutant samples in this case made it possible to distinguish samples that contained a mix of both mutant and consensus genotype cells (FIG. 38). These predictably occurred on patch boundaries. In addition to 9 cancerous biopsies, a mutation in marker 47 identified two histologically negative and two non-cancer dysplasia samples as belonging to the same clone, suggesting that the cancer and dysplasia emerged from a pre-existing, nondysplastic clonal field.

### Discussion

This example shows that neutral markers of cell lineage can be used to identify clonal expansions in non-dysplastic UC colon and effectively distinguish patients who have progressed to histologically advanced disease from those who have not. These multifocal clones may occur as fields from which a cancer has arisen or as otherwise invisible populations, more than half a meter away from the nearest dysplasia. These findings reinforce prior evidence that neoplastic evolution in UC is delocalized, multifocal and involves both epithelium and stroma (Risques RA et al., Curr Opin Gastroenterol (2006) 22(4):382-390; Brentnall TA, et al., Gastroenterology (1994) 107(2):369-378; Burmer GC, et al., Gastroenterology (1992) 103(5):1602-1610; Rubin C E, et al., Gastroenterology (1992) 103(5):1611-1620; Rabinovitch PS, et al., Cancer Res (1999) 59(20):5148-5153; Chen R et al., Cancer Genet Cytogenet (2005) 162(2):99-106; Leedham S, et al., Gastroenterology (2009) 136(2):542-550; Yagishita H et al., Scand. J. of Gastroenterology (2008) 43(5):559-566; 7, 15-19, 31, 32). These results suggest that screening for clonal expansions in UC patients may be able to identify tumorogenic processes prior to the emergence of histologically recognizable disease.

The presence of large, clonally-derived patches in colon represent a divergence from normal cellular homeostasis. The colon is divided into replicative units known as crypts. These invaginated structures contain a small population of stem cells at their base that continually replicate to clonally populate the luminal surfaces with terminally differentiated progeny that are then sloughed off after several days (Leedham S J & Wright N A, Journal of Clinical Pathology (2008) 61(2):164-171). Studies of normal female colons have shown that embryonically-originating patches sharing a common inactivated X-chromosome do not exceed 450 crypts in size (Novelli M, et al., Proc Natl Acad Sci USA (2003) 100(6):3311-3314). Spontaneously arising mitochondrial mutations indicate that clonal crypt clusters arise post-natally in normal colon and increase in size with age, yet rarely exceed a dozen units (Greaves L C, et al., Proc Natl Acad Sci USA (2006) 103(3):714-719). The biopsy portions we genotyped contained approximately 2000-5000 crypts. Because at least 40% of cells in a sample must share a mutation for it to be detectable, it can be estimated that clones within isolated mutant biopsies comprise at least 800-2000 crypts. In this study only one patch was identified in a single Non-Progressor colon. It is possible that this case may, in fact, represent one of the 10% of the general UC population who would have ultimately progressed to cancer.

Using neutral passenger rather than putative driver mutations as lineage markers of clonal expansions has two primary advantages. First, cancer is a stochastic evolutionary process, and not all tumors necessarily arise through mutation of the same set of drivers (Fox E J et al., Cancer Res (2009) 69(12):4948-4950; Salk JJ et al., Ann Rev Path (2010) 5 (In press)). Neutral mutations offer an unbiased, generalizable way of identifying clones that is independent of molecular causation. Such an approach integrates all possible bases for an abnormal cell/tissue behavior, including mutation of unknown genetic and epigenetic sites as well as non-heritable influences of local environment. Second, screening for known drivers may limit detectability to relatively late-arising clones. Experimental mutation of many of the best characterized oncogenes and tumor suppressors in otherwise untransformed cells induces growth arrest and senescence (Ben-Porath I & Weinberg R A, Int J Biochem Cell Biol (2005) 37(5):961-976). It is conceivable that mutation in common tumor drivers seen in UC-derived adenocarcinoma, including TP53 and KRAS, may not be tolerated in the earliest arising clones. Similar arguments mitigate against the utility of randomly-arising gross chromosomal abnormalities as early clonal markers.

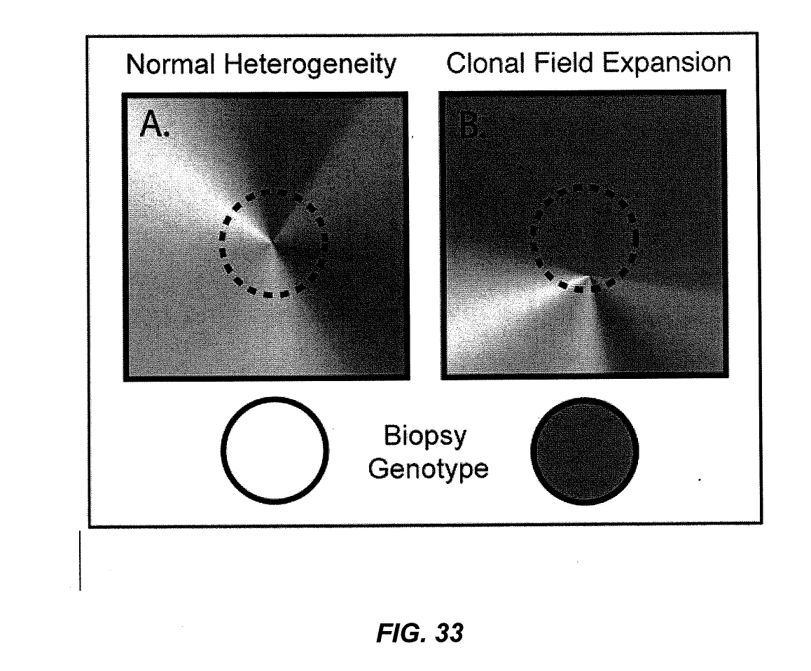

Functionally neutral mutations serve as useful markers of clonality but are, nevertheless, imperfect. Detection of a clone requires that members share a common mutation, originating in the founding cell, which distinguishes them from the surrounding population (FIG. 33). While every clone is likely to be uniquely marked relative to neighboring cells somewhere in the >400,000 polyguanine tracts in the human genome, there is no guarantee that a mutation will have occurred in the specific subset of tracts being genotyped so some clones will be undetectable. Although we identified clones in ⅔rds of Progressor biopsies, the true proportion is likely to be even greater given that we only examined 28 polyguanine tracts.

Although this study relies on microsatellite mutations as lineage markers, we find no evidence for the presence of the extensive microsatellite instability (MSI) that results from deficiencies in mismatch repair (MMR) (Boland CR, et al. Cancer Res (1998) 58(22):5248-5257). No mutations were identified in any of the NCI Bethesda Panel MSI markers examined, and the low frequency of polyguanine mutations detected (1.4% overall, 2.3% in Progressors) is inconsistent with a vastly increased rate of microsatellite slippage (Boyer JC, et al. Hum Mol Genet (2002) 11(6):707-713). Moreover, whereas MMR-mediated MSI commonly manifests as a “widening” of electropherogram stutter peaks, indicative of a large number of length variants simultaneously present within a sample (Tsao J L, et al., Proc Natl Acad Sci USA 9 (2000) 7(3):1236-1241), the rare polyguanine mutations we identified were predominantly the result of single slipped alleles. While there have been reports of “low-level” MSI in UC-derived adenocarcinomas and occasionally in surrounding non-dysplastic tissue (Yagishita H et al., Scand. J. of Gastroenterology (2008) 43(5):559-566; Brentnall TA, et al., Cancer Res (1996) 56(6):1237-1240; Ozaki K, et al., Int J Cancer (2006) 119(11):2513-2519), microsatellite slippage events are expected to be occasionally witnessed in any clonally-derived population (Salipante SJ & Horwitz M S, Proc Natl Acad Sci USA (2006) 103(14):5448-5453; Graham T et al., J. Pathol. (2008) 215(2):204-210), and the latter may best explain our findings. Nevertheless, it is possible that a moderately increased rate of slippage may occur in UC. Direct measurement of the per-division mutation rate in UC Progressors relative to normal colon would be challenging given that detection of slippage is necessarily coupled to clonal expansion-something that does not occur in normal colon. Although an increased rate of mutagenesis resulting from inflammation-derived reactive oxygen species (Jackson A L et al., Proc Natl Acad Sci USA (1998) 95(21):12468-12473; Hofseth L J, et al. J Clin Invest (2003) 112(12):1887-1894) or defects in replication fidelity acquired during neoplastic transformation (Loeb L A et al., Cancer Res (1974) 34(9):2311-2321) would increase the number of somatic variants and facilitate detection of clones, it is not mandatory to invoke such mechanisms to explain the present observations.

A unique aspect of this study is that it exclusively relies on neutral markers of cell lineage to identify clinically-relevant clonal expansions in a preneoplastic condition. Several groups have previously used neutral markers to study tumor ancestry and tissue dynamics. In combination with suspected driver mutations, Maley and colleagues used slippage in short tandem repeats to show that risk of cancer progression is related to clonal diversity in Barrett's esophagus (Maley CC, et al., Cancer Res (2004) 64(20):7629-7633). Although the bulk of clones in the study were identified by likely-driver mutations, evolutionary statistics suggested that clonal heterogeneity, in- and -of itself, is an important cancer predictor. Frumkin et al. used microsatellite slippage events to phylogenetically reconstruct the lineage relationship between single cells in a mouse lymphoma (Frumkin D, et al., Cancer Res (2008) 68(14):5924-5931). Shibata's group employed non-coding microsatellite mutations as a “molecular clock” of somatic division and used this to calculate the number of cell generations between initiation and sampling of human MSI tumors (Tsao J L, et al., Proc Natl Acad Sci USA 9 (2000) 7(3):1236-1241). More recently these investigators have capitalized on the high frequency of epimutations in CpG islands of non-transcribed genes in order to study the clonal structure of non-MSI tumor development (Siegmund K et al., Cell Cycle (2009) 8(14)). Wright and colleagues have recently used the stainable phenotype of neutral, homoplasmic mutations in a mitochondrially-encoded cyclooxygenase gene to study cell lineages in variety of tissues (Fellous T, et al. Stem Cells (2009) 27(6):1410-1420). Although the process by which homoplasmy arises is not well understood and takes many years to develop in some tissues (Greaves LC, et al., Proc Natl Acad Sci USA (2006) 103(3):714-719), the ability to directly visualize the individual cells of a clonal population in an unperturbed tissue context makes this a promising technique with the potential to complement the present studies.

The experiments undertaken herein expand on previous studies of field effects in UC. It is shown herein that neutral markers of cell lineage can identify large clonally-derived patches in normal-appearing colon of patients having histologically recognizable disease but not in those without. Although this ability will be of clinical utility in its own right, a more significant possibility is that such patches may be able to identify individual UC patients at greatest risk for developing colon cancer prior to the emergence of histological changes (i.e., future Progressors). The present observation that clones may cover an area with both dysplastic and non-dysplastic tissue strongly argues that expansions can predate dysplasia. Prospective studies will be needed to determine efficacy as a predictive biomarker of cancer risk.

From a basic science perspective, we have demonstrated that randomly occurring mutations can be used to define the boundaries of clonal expansions occurring in vivo and identify subclones within larger clones. Screening a larger number of polyguanine marker sites will enable detailed phylogenetic reconstruction of the relationship between clones and may allow estimation of the number of rounds of selective outgrowths needed for neoplastic transformation. After using these markers to define clone boundaries, it will be possible to screen for candidate driver mutations and determine the fraction of expansions that are driven by factors other than genes already known to play a role in UC-mediated carcinogenesis. The methods provided in certain aspects of the present invention technique should be adaptable to the study of other preneoplastic conditions or cancers. The general tactic of tracing cell lineage with spontaneously arising neutral markers of all forms holds promise for better understanding the neoplastic process. While there are at least 400,000 polyguanine tracts in the human genome, there are billions other nucleotides and epigenetic sites for which mutation is likely to be functionally silent. When it becomes technically and economically possible to screen the whole genome with future sequencing technologies, a wealth of developmental history from all forms of normal and abnormal cell proliferation will become available. Not only do random mutations form the fundamental basis of evolutionary biology, they provide a powerful tool for studying its role in human development and disease.

### Materials and Methods:

For initial studies, biopsies were harvested from the colons of 19 UC patients: 11 with cancer or high-grade dysplasia (Progressors) and 8 without (Non-Progressors). Samples were obtained either at colonoscopy (3 UC Non-Progressors, 1 UC Progressor) or immediately following colectomy (5 UC Non-Progressors, 7 UC Progressors) in accordance with approved Human Subject's Guidelines at the University of Washington. Colectomy specimens came from surgeries performed for intractable symptoms, emergent hemorrhage, sepsis or the presence of high-grade dysplasia or cancer found during colonoscopy. Progression status was determined by histological assessment of 44-144 biopsies depending on whether samples were obtained at colonoscopy or colectomy. Biopsies were evenly spaced along the colon with diagnosis for dysplasia made according to DMSG consensus criteria (Riddell RH, et al. Hum Pathol (1983) 14(11):931-968) by a GI pathologist (author MPB). Selection of colons was based on surgical availability. All samples were frozen at −70° C. in Minimal Essential Medium with 10% DMSO until use. The mean patient age within each group was: Non-Progressors: 45.0 (range: 23-59) and Progressors: 41.2 (range: 31-61). The mean year-duration of UC within each group was: Non-Progressors: 10.8 (range: 0.25-20), Progressors: 15.5 (range: 4-29). Additional patient information is listed in SI 1. For the whole-colon mapping study, biopsies taken at time of colectomy were obtained from a UC Progressor of unknown age and disease duration. In all cases, colonic epithelial cells were isolated from a ˜9 mm² portion of each biopsy by EDTA shakeoff; this method yields>90% enrichment for epithelial cells (Rabinovitch PS, et al. (1999) Cancer Res 59(20):5148-5153). DNA from the epithelial cell isolate and residual stroma of each biopsy was extracted by silica filtration column (Qiagen) and quantified by Nanodrop UV spectroscopy (Thermo Scientific). Stromal DNA was not included in the latter two mapping studies An adjacent portion of each biopsy was fixed and sectioned for histology.

### Polyguanine Tract-Length Genotyping

A BLAST search (Altschul SF, et al. Nucleic Acids Research (1997) 25(17):3389-3402) of NCBI build 36 of the human genome was used to identify polyguanine tracts of 12 or more residues in length. Flanking oligonucleotide primers (FIG. 34) were synthesized (Operon or Applied Biosystems). All forward primers integrated a 5′ fluorescent dye (6-FAM, NED or HEX), and reverse primers contained GTTTCTT on the 5′ end to minimize genotyping artifacts due to the terminal deoxynucleotidyl transferase activity of Taq DNA polymerase (Brownstein M J et al., BioTechniques (1996) 20(6):1004-1006, 1008-1010). PCR reactions (5 μl) containing 4 ng genomic DNA were prepared in robotically-loaded 384 well plates, and amplification was carried out for 42 cycles using Taq DNA polymerase (Qiagen). PCR fragments were resolved with an ABI PRISM 3730xl Genetic Analyzer. All genotypes were repeated in duplicate. Electropherograms were analyzed with ABI GeneMapper 4.0 software in a fully blinded fashion using the PeakSeeker approach (Thompson J M & Salipante SJ, BMC Res Notes (2009) 2:17). For each marker, the allele lengths most commonly observed in a set of samples derived from a single patient was defined as the consensus genotype. Genotypes differing from the consensus were considered mutant. If both replicates for a sample produced robust signals and yielded the same genotype, or if only one replicate generated good signal and was equal to the consensus genotype, the result was recorded. If only one replicate produced sufficient signal and suggested a mutant genotype, or if there was a discrepancy between genotypes of the two replicates, the allele lengths were resolved using three additional PCR amplifications. If neither replicate produced a robust signal, the genotype was marked as “X” and not repeated. Some amplification failures were sporadic; others were systematically related to certain PCR amplicons. Progressor and Non-Progressor DNA samples were handled identically in that they were amplified in intermingled wells on the same PCR plates and genotyped blindly.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of identifying the presence of a precancerous field in a subject, the method comprising detecting a discrete change in the size of a genomic poly-G tract in a biological sample from the subject.

2. The method of claim 1, wherein the subject has been diagnosed with a gastrointestinal disorder.

3. The method of claim 2, wherein the disease is ulcerative colitis or Crohn's disease.

4. The method of claim 1, wherein the genomic poly-G tract is selected from the group consisting of the loci found in Table 1.

5. The method of claim 1, wherein the biological sample is a biopsy from the bowel of the subject.

6. The method of claim 5, wherein the method comprises the use of 10 or fewer biopsies.

7. The method of claim 1, wherein the presence of a precancerous field indicates that the subject has an increased likelihood of developing cancer.

8. The method of claim 7, wherein the cancer is colon cancer.

9. A method of providing a prognosis for a subject diagnosed with a chronic inflammatory bowel disease, the method comprising the steps of:
(a) isolating genomic DNA from a plurality of biopsies from the subject;
(b) determining the length of a poly-G tract in the biopsies; and
(c) comparing the lengths of the poly-G tract between the different biopsies, wherein a difference in the length of a poly-G tract between two biopsies indicates that the subject has an increased risk of developing cancer, thereby providing a prognosis for the subject.

10. A method of assigning a course of treatment to a subject diagnosed with a chronic inflammatory bowel disease, the method comprising the steps of:
(a) determining the length of a poly-G tract in a plurality of biopsies from the bowel of the subject;
(b) detecting a difference in the length of a poly-G tract in a first biopsy from the subject as compared to a second biopsy from the subject; and
(c) assigning a course of treatment.

11. The method of claim 10, wherein the course of treatment is further diagnostic evaluation comprising a colonoscopy with biopsy.

12. The method of claim 10, wherein the course of treatment assigned to a subject having an altered poly-G tract comprises bowel resection surgery.

13. The method of claim 12, wherein the method further comprises determining the length of bowel to remove by:
(d) removing additional biopsies proximal to the biopsy having an altered poly-G tract; and
(e) determining the length of the poly-G tract in the additional biopsies, thereby determining the boundaries of the bowel to remove.

14. A kit for diagnosing or providing a prognosis for an increased risk of developing cancer in a subject, the kit comprising a polynucleotide primer that may be extended to amplify a poly-G locus selected from those found in Table 1.

15. A solid-state platform for diagnosing or providing a prognosis of an increased risk of developing cancer in an individual, the platform comprising a plurality of nucleic acids that hybridize to nucleic acid sequences from at least two of the poly-G tract loci found in Table 1.

16. A method of identifying the presence of a precancerous field in a subject having an increased risk of developing cancer, the method comprising detecting a discrete change in the size of a repeated genomic tract in a biological sample taken from the bowel of the subject.

17. The method of claim 16, wherein the repeated genomic tract is a dinucleotide repeat, a trinucleotide repeat, or a higher order nucleotide repeat.

18. The method of claim 16, wherein the cancer is selected from the group consisting of lung cancer, oropharyngeal cancer, biliary tract cancer, and uterine cancer.

19. A method of assessing the adequacy of surgical margins used for a surgical resection, the method comprising the steps of:
(a) isolating a sample proximal to the site of a surgical resection; and
(b) detecting a discrete change in the size of a genomic poly-G tract in the sample,
wherein the presence of a change in the size of a genomic poly-G tract in the sample indicates that the surgical margins were not adequate.

20. The method of claim 19, wherein said method is performed during or directly after Mohs surgery.
